(19) United States
(12) Patent Application Publication
(10) Pub. No.: US 2022/0162606 A1
(43) Pub. Date: May 26, 2022

(54) NRL EXPRESSION REDUCING OLIGONUCLEOTIDES, COMPOSITIONS CONTAINING THE SAME, AND METHODS OF THEIR USE

(71) Applicant: Nayan Therapeutics, Inc., Medfield, MA (US)

(72) Inventor: Gerald SEWACK, Needham, MA (US)

(21) Appl. No.: 17/425,529

(22) PCT Filed: Jan. 24, 2020
(86) PCT No.: PCT/US2020/015095

§ 371 Date: Jul. 23, 2021

Related U.S. Application Data
(60) Provisional application No. 62/796,939, filed on Jan. 25, 2019.
(60) Provisional application No. 62/796,987, filed on Jan. 25, 2019.
(60) Provisional application No. 62/797,027, filed on Jan. 25, 2019.

(51) Int. Cl. C12N 15/113

(52) U.S. Cl. CPC C12N 15/113; C12N 2310/11

(57) ABSTRACT

Disclosed are oligonucleotides having a nucleobase sequence with at least 6 contiguous nucleobases complementary to an equal-length portion within an NRL target nucleic acid. The oligonucleotides may be single-stranded or double-stranded. Also disclosed are pharmaceutical compositions containing the oligonucleotides and methods of their use.

### SEQUENCE LISTING

The instant application contains a Sequence Listing which has been submitted electronically in ASCII format and is hereby incorporated by reference in its entirety. Said ASCII copy, created on Jan. 24, 2020 is named 51367-007 WO2_Sequence_Listing_01.24.20_ST25 and is 75,933 bytes in size.

### FIELD OF THE INVENTION

The invention provides oligonucleotides, compositions containing the same, and methods of their use.

### BACKGROUND

Retinitis pigmentosa is a group of inherited, progressive diseases causing retinal degeneration. Patients having retinitis pigmentosa experience a gradual decline in their vision because photoreceptor cells in the retina degenerate.

In most forms of retinitis pigmentosa, rod cells are affected first. Because rods are concentrated in outer portions of the retina and are triggered by dim light, their degeneration affects peripheral and night vision. When the disease progresses and cones become affected, visual acuity, color perception, and central vision are diminished. Night blindness is one of the earliest and most frequent symptoms of retinitis pigmentosa. On the other hand, patients with cone degeneration first experience decreased central vision and reduced ability to discriminate colors and perceive details.

Retinitis pigmentosa is typically diagnosed in adolescents and young adults. The rate of progression and degree of visual loss varies from person to person. Most people with retinitis pigmentosa are legally blind by age 40 with a central visual field of less than 20 degrees in diameter.

There is currently no cure for retinitis pigmentosa. Applicability of various supplements, such as vitamin A, docosahexaenoic acid, and lutein, to slow the progression of retinitis pigmentosa remain largely unresolved. Currently, the main marketed treatment for retinitis pigmentosa is an electronic retinal implant. This treatment approach, however, requires intraocular, surgical implantation and is prosthetic by design. Therefore, it does not prevent the loss of rod and cone cells underlying the symptoms of retinitis pigmentosa.

There is a need for new therapeutic approaches to the treatment of retinitis pigmentosa.

### SUMMARY OF THE INVENTION

In general, the invention provides oligonucleotides including a nucleobase sequence including at least 6 contiguous nucleobases complementary to an equal-length portion within a NRL target nucleic acid. The invention also provides compositions containing oligonucleotides of the invention and methods of using the same.

In one aspect, the invention provides a single-stranded oligonucleotide including a total of 12 to 50 interlinked nucleotides and having a nucleobase sequence including at least 6 contiguous nucleobases complementary to an equal-length portion within a NRL target nucleic acid.

In some embodiments, the oligonucleotide includes at least one modified nucleobase. In certain embodiments, at least one modified nucleobase is 5-methylcytosine. In particular embodiments, at least one modified nucleobase is 7-deazaguanine. In further embodiments, at least one modified nucleobase is 6-thioguanine.

In yet further embodiments, the oligonucleotide includes at least one modified internucleoside linkage. In still further embodiments, the modified internucleoside linkage is a phosphorothioate linkage. In certain embodiments, the phosphorothioate linkage is a stereochemically enriched phosphorothioate linkage. In some embodiments, at least 50% of internucleoside linkages in the oligonucleotide are each independently the modified internucleoside linkage. In certain embodiments, at least 70% of internucleoside linkages in the oligonucleotide are each independently the modified internucleoside linkage.

In particular embodiments, the oligonucleotide includes at least one modified sugar nucleoside. In further embodiments, at least one modified sugar nucleoside is a bridged nucleic acid. In yet further embodiments, the bridged nucleic acid is a locked nucleic acid (LNA), ethylene-bridged nucleic acid (ENA), or cEt nucleic acid. In still further embodiments, the oligonucleotide is a gapmer, headmer, or tailmer. In some embodiments, at least one modified sugar nucleoside is a 2′-modified sugar nucleoside. In certain embodiments, at least one 2′-modified sugar nucleoside includes a 2′-modification selected from the group consisting of 2′-fluoro, 2′-methoxy, and 2′-methoxyethoxy. In particular embodiments, the oligonucleotide includes deoxyribonucleotides. In further embodiments, the oligonucleotide includes ribonucleotides. In yet further embodiments, the oligonucleotide is a morpholino oligomer.

In still further embodiments, the oligonucleotide includes a hydrophobic moiety covalently attached at a 5′-terminus, 3′-terminus, or internucleoside linkage of the oligonucleotide.

In certain embodiments, the NRL target nucleic acid is NRL transcript 1. In particular embodiments, the NRL target nucleic acid is NRL transcript 2. In some embodiments, the NRL target nucleic acid is NRL transcript 3. In further embodiments, the NRL target nucleic acid is NRL transcript 4. In yet further embodiments, the oligonucleotide includes a region complementary to a region within the sequence from position 547 to position 1260 in NRL transcript 1. In still embodiments, the oligonucleotide includes a region complementary to a region within the sequence from position 354 to position 753 in NRL transcript 1. In some embodiments, the oligonucleotide includes a region complementary to a region within the sequence from position 569 to position 634 in NRL transcript 1. In certain embodiments, the oligonucleotide includes a region complementary to a region within the sequence from position 807 to position 866 in NRL transcript 1. In particular embodiments, the oligonucleotide includes a region complementary to a region within the sequence from position 1149 to position 1260 in NRL transcript 1. In further embodiments, the oligonucleotide includes a region complementary to a region within the sequence from position 888 to position 911 in NRL transcript 1. In yet further embodiments, the oligonucleotide includes a nucleobase sequence including at least 6 contiguous nucleobases complementary to a region including a sequence selected from the group consisting of positions 642-645, 766-769, and 1127-1130 in NRL transcript 1. In still further embodiments, the oligonucleotide includes a nucleobase sequence including at least 6 contiguous nucleobases complementary to a region including a sequence selected from the group consisting of positions 892-895, 974-977, 1175-1178, and 1235-1238 in NRL transcript 1. In some embodiments, the oligonucleotide includes a nucleobase sequence including at least 6 contiguous nucleobases complementary to a region including a sequence of positions 721-724 in NRL transcript 1. In certain embodiments, the oligonucleotide includes a nucleobase sequence including at least 6 contiguous nucleobases complementary to a region including a sequence of positions 904-907 in NRL transcript 1. In particular embodiments, the oligonucleotide includes a nucleobase sequence including at least 6 contiguous nucleobases complementary to a region including a sequence selected from the group consisting of positions 825-828, 933-936, and 1031-1034 in NRL transcript 1.

In some embodiments, the oligonucleotide includes at least 8 contiguous nucleobases complementary to an equal-length portion within a NRL target nucleic acid. In certain embodiments, the oligonucleotide includes at least 12 contiguous nucleobases complementary to an equal-length portion within a NRL target nucleic acid. In particular embodiments, the oligonucleotide includes 20 or fewer contiguous nucleobases complementary to an equal-length portion within the NRL target nucleic acid.

In further embodiments, the oligonucleotide includes a total of at least 12 interlinked nucleotides. In yet further embodiments, the oligonucleotide includes a total of 24 or fewer interlinked nucleotides.

In another aspect, the invention provides a double-stranded oligonucleotide including an oligonucleotide of the invention hybridized to a complementary oligonucleotide. In some embodiments, the complementary oligonucleotide has the same length as the oligonucleotide of the invention. In further embodiments, the complementary oligonucleotide has a length that is ±1, ±2, ±3, ±4, or ±5 nucleotides relative to the number of nucleotides in the oligonucleotide of the invention.

In another aspect, the invention provides a double-stranded oligonucleotide including a passenger strand hybridized to a guide strand including a nucleobase sequence including at least 6 contiguous nucleobases complementary to an equal-length portion within a NRL target nucleic acid. In certain embodiments, each of the passenger strand and the guide strand includes a total of 12 to 50 interlinked nucleotides.

In some embodiments, the passenger strand includes at least one modified nucleobase. In particular embodiments, at least one modified nucleobase is 5-methylcytosine. In further embodiments, at least one modified nucleobase is 7-deazaguanine. In yet further embodiments, at least one modified nucleobase is 6-thioguanine.

In still further embodiments, the passenger strand includes at least one modified internucleoside linkage. In some embodiments, the modified internucleoside linkage is a phosphorothioate linkage. In certain embodiments, the phosphorothioate linkage is a stereochemically enriched phosphorothioate linkage. In particular embodiments, at least 50% of internucleoside linkages in the passenger strand are each independently the modified internucleoside linkage. In further embodiments, at least 70% of internucleoside linkages in the passenger strand are each independently the modified internucleoside linkage.

In certain embodiments, the passenger strand includes at least one modified sugar nucleoside. In some embodiments, at least one modified sugar nucleoside is a bridged nucleic acid. In particular embodiments, the bridged nucleic acid is a locked nucleic acid (LNA), ethylene-bridged nucleic acid (ENA), or cEt nucleic acid. In further embodiments, at least one modified sugar nucleoside is a 2′-modified sugar nucleoside. In yet further embodiments, at least one 2′-modified sugar nucleoside includes a 2′-modification selected from the group consisting of 2′-fluoro, 2′-methoxy, and 2′-methoxyethoxy. In still further embodiments, the passenger strand includes deoxyribonucleotides. In certain embodiments, the passenger strand includes ribonucleotides.

In particular embodiments, the passenger strand includes a hydrophobic moiety covalently attached at a 5′-terminus, 3′-terminus, or internucleoside linkage of the passenger strand.

In some embodiments, the guide strand includes at least one modified nucleobase. In further embodiments, at least one modified nucleobase is 5-methylcytosine. In yet further embodiments, at least one modified nucleobase is 7-deazaguanine. In still further embodiments, at least one modified nucleobase is 6-thioguanine.

In certain embodiments, the guide strand includes at least one modified internucleoside linkage. In some embodiments, the modified internucleoside linkage is a phosphorothioate linkage. In particular embodiments, the phosphorothioate linkage is a stereochemically enriched phosphorothioate linkage. In further embodiments, at least 50% of internucleoside linkages in the guide strand are each independently the modified internucleoside linkage. In yet further embodiments, at least 70% of internucleoside linkages in the guide strand are each independently the modified internucleoside linkage.

In still further embodiments, the guide strand includes at least one modified sugar nucleoside. In some embodiments, at least one modified sugar nucleoside is a bridged nucleic acid. In certain embodiments, the bridged nucleic acid is a locked nucleic acid (LNA), ethylene-bridged nucleic acid (ENA), or cEt nucleic acid. In particular embodiments, at least one modified sugar nucleoside is a 2′-modified sugar nucleoside. In further embodiments, at least one 2′-modified sugar nucleoside includes a 2′-modification selected from the group consisting of 2′-fluoro, 2′-methoxy, and 2′-methoxyethoxy. In yet further embodiments, the guide strand includes deoxyribonucleotides. In still further embodiments, the guide strand includes ribonucleotides.

In some embodiments, the guide strand includes a hydrophobic moiety covalently attached at a 5′-terminus, 3′-terminus, or internucleoside linkage of the passenger strand. In certain embodiments, the guide strand includes a region complementary to a coding sequence within the NRL target nucleic acid. In particular embodiments, the NRL target nucleic acid is NRL transcript 1. In further embodiments, the NRL target nucleic acid is NRL transcript 2. In yet further embodiments, the NRL target nucleic acid is NRL transcript 3. In still further embodiments, the NRL target nucleic acid is NRL transcript 4. In some embodiments, the guide strand includes a sequence complementary to a sequence including positions 586-605 or 264-283 in NRL transcript 1. In certain embodiments, the guide strand includes a sequence complementary to a sequence including positions 815-834 or 965-984 in NRL transcript 1.

In certain embodiments, the hybridized oligonucleotide includes at least one 3′-overhang (e.g., 1, 2, 3, or 4 nucleotide-long overhang; e.g., UU overhang). In particular embodiments, the hybridized oligonucleotide is a blunt. In some embodiments, the hybridized oligonucleotide includes two 3′-overhangs (e.g., 1, 2, 3, or 4 nucleotide-long overhang; e.g., UU overhang).

In a yet another aspect, the invention provides a pharmaceutical composition including the oligonucleotide of the invention and a pharmaceutically acceptable excipient.

In a still another aspect, the invention provides methods of use of the oligonucleotides of the invention.

In some embodiments, the method is a method of inhibiting the production of an NRL protein in a cell including (e.g., expressing) an NRL gene by contacting the cell with the oligonucleotide of the invention.

In certain embodiments, the cell is in a subject. In particular embodiments, the cell is in the subject's eye.

In further embodiments, the method is a method of treating a subject in need thereof by administering to the subject a therapeutically effective amount of the oligonucleotide of the invention or the pharmaceutical composition of the invention.

In yet further embodiments, the oligonucleotide or pharmaceutical composition is administered intraocularly or topically to the eye of the subject. In still further embodiments, the subject is in need of a treatment for an ocular disease, disorder, or condition associated with a dysfunction of ABCA4, AIPL1, BBS1, BEST1, CEP290, CDH3, CHM, CNGA3, CNGB3, CRB1, GUCY2D, MERTK, MRFP, MYO7A, ND4, NR2E3, PDE6, PRPH2, RD3, RHO, RLBP1, RP1, RPE65, RPGR, RPGRIP1, RS1, or SPATA7 gene. In some embodiments, the subject is in need of a treatment for retinitis pigmentosa, Stargardt disease, cone-rod dystrophy, Leber congenital amaurosis, Bardet Biedl syndrome, macular dystrophy, dry macular degeneration, geographic atrophy, atrophic age-related macular degeneration (AMD), advanced dry AMD, retinal dystrophy, choroideremia, Usher syndrome type 1, retinoschisis, Leber hereditary optic neuropathy, and achromatopsia. In preferred embodiments, the subject is in need of a treatment for retinitis pigmentosa. In certain embodiments, retinitis pigmentosa is Rho P23H-associated retinitis pigmentosa, PDE6-associated retinitis pigmentosa, MERTK-associated retinitis pigmentosa, BBS1-associated retinitis pigmentosa, Rho-associated retinitis pigmentosa, MRFP-associated retinitis pigmentosa, RLBP1-associated retinitis pigmentosa, RP1-associated retinitis pigmentosa, RPGR-X-linked retinitis pigmentosa, NR2E3-associated retinitis pigmentosa, or SPATA7-associated retinitis pigmentosa.

The invention is also described by the following enumerated items.

1. A single-stranded oligonucleotide comprising a total of 12 to 50 interlinked nucleotides and having a nucleobase sequence comprising at least 6 contiguous nucleobases complementary to an equal-length portion within an NRL target nucleic acid.
2. The oligonucleotide of item 1, wherein the oligonucleotide comprises at least one modified nucleobase.
3. The oligonucleotide of item 2, wherein at least one modified nucleobase is 5-methylcytosine.
4. The oligonucleotide of item 2 or 3, wherein at least one modified nucleobase is 7-deazaguanine.
5. The oligonucleotide of any one of items 2 to 4, wherein at least one modified nucleobase is 6-thioguanine.
6. The oligonucleotide of any one of items 1 to 5, wherein the oligonucleotide comprises at least one modified internucleoside linkage.
7. The oligonucleotide of item 6, wherein the modified internucleoside linkage is a phosphorothioate linkage.
8. The oligonucleotide of item 7, wherein the phosphorothioate linkage is a stereochemically enriched phosphorothioate linkage.
9. The oligonucleotide of any one of items 6 to 8, wherein at least 50% of internucleoside linkages in the oligonucleotide are each independently the modified internucleoside linkage.
10. The oligonucleotide of item 9, wherein at least 70% of internucleoside linkages in the oligonucleotide are each independently the modified internucleoside linkage.
11. The oligonucleotide of any one of items 1 to 10, wherein the oligonucleotide comprises at least one modified sugar nucleoside.
12. The oligonucleotide of item 11, wherein at least one modified sugar nucleoside is a bridged nucleic acid.
13. The oligonucleotide of item 12, wherein the bridged nucleic acid is a locked nucleic acid (LNA), ethylene-bridged nucleic acid (ENA), or cEt nucleic acid.
14. The oligonucleotide of item 13, wherein the oligonucleotide is a gapmer.
15. The oligonucleotide of any one of items 11 to 14, wherein at least one modified sugar nucleoside is a 2′-modified sugar nucleoside.
16. The oligonucleotide of item 15, wherein at least one 2′-modified sugar nucleoside comprises a 2′-modification selected from the group consisting of 2′-fluoro, 2′-methoxy, and 2′-methoxyethoxy.
17. The oligonucleotide of any one of items 1 to 16, wherein the oligonucleotide comprises deoxyribonucleotides.
18. The oligonucleotide of any one of items 1 to 17, wherein the oligonucleotide comprises ribonucleotides.
19. The oligonucleotide of any one of items 1 to 5, wherein the oligonucleotide is a morpholino oligomer.
20. The oligonucleotide of any one of items 1 to 19, wherein the oligonucleotide comprises a hydrophobic moiety covalently attached at a 5′-terminus, 3′-terminus, or internucleoside linkage of the oligonucleotide.
21. The oligonucleotide of any one of items 1 to 20, wherein the oligonucleotide comprises a region complementary to a coding sequence within the NRL target nucleic acid.
22. The oligonucleotide of any one of items 1 to 21, wherein the NRL target nucleic acid is NRL transcript 1.
23. The oligonucleotide of any one of items 1 to 21, wherein the NRL target nucleic acid is NRL transcript 2.
24. The oligonucleotide of any one of items 1 to 21, wherein the NRL target nucleic acid is NRL transcript 3.
25. The oligonucleotide of any one of items 1 to 21, wherein the NRL target nucleic acid is NRL transcript 4.
26. The oligonucleotide of any one of items 1 to 20, wherein the oligonucleotide comprises a region complementary to a region within the sequence from position 547 to position 1260 in NRL transcript 1.
27. The oligonucleotide of any one of items 1 to 20, wherein the oligonucleotide comprises a region complementary to a region within the sequence from position 354 to position 753 in NRL transcript 1.
28. The oligonucleotide of any one of items 1 to 20, wherein the oligonucleotide comprises a region complementary to a region within the sequence from position 569 to position 634 in NRL transcript 1.
29. The oligonucleotide of any one of items 1 to 20, wherein the oligonucleotide comprises a region complementary to a region within the sequence from position 807 to position 866 in NRL transcript 1.
30. The oligonucleotide of any one of items 1 to 20, wherein the oligonucleotide comprises a region complementary to a region within the sequence from position 1149 to position 1260 in NRL transcript 1.
31. The oligonucleotide of any one of items 1 to 20, wherein the oligonucleotide comprises a region complementary to a region within the sequence from position 888 to position 911 in NRL transcript 1.
32. The oligonucleotide of any one of items 1 to 20, wherein the oligonucleotide comprises a nucleobase sequence comprising at least 6 contiguous nucleobases complementary to a region comprising a sequence selected from the group consisting of positions 642-645, 766-769, and 1127-1130 in NRL transcript 1.
33. The oligonucleotide of any one of items 1 to 20, wherein the oligonucleotide comprises a nucleobase sequence comprising at least 6 contiguous nucleobases complementary to a region comprising a sequence selected from the group consisting of positions 892-895, 974-977, 1175-1178, and 1235-1238 in NRL transcript 1.
34. The oligonucleotide of any one of items 1 to 20, wherein the oligonucleotide comprises a nucleobase sequence comprising at least 6 contiguous nucleobases complementary to a region comprising a sequence of positions 721-724 in NRL transcript 1.
35. The oligonucleotide of any one of items 1 to 20, wherein the oligonucleotide comprises a nucleobase sequence comprising at least 6 contiguous nucleobases complementary to a region comprising a sequence of positions 904-907 in NRL transcript 1.
36. The oligonucleotide of any one of items 1 to 20, wherein the oligonucleotide comprises a nucleobase sequence comprising at least 6 contiguous nucleobases complementary to a region comprising a sequence selected from the group consisting of positions 825-828, 933-936, and 1031-1034 in NRL transcript 1.
37. The oligonucleotide of any one of items 1 to 32, wherein the oligonucleotide comprises at least 8 contiguous nucleobases complementary to an equal-length portion within an NRL target nucleic acid.
38. The oligonucleotide of any one of items 1 to 32, wherein the oligonucleotide comprises at least 12 contiguous nucleobases complementary to an equal-length portion within an NRL target nucleic acid.
39. The oligonucleotide of any one of items 1 to 34, wherein the oligonucleotide comprises 20 or fewer contiguous nucleobases complementary to an equal-length portion within the NRL target nucleic acid.
40. The oligonucleotide of any one of items 1 to 35, wherein the oligonucleotide comprises a total of at least 12 interlinked nucleotides.
41. The oligonucleotide of any one of items 1 to 36, wherein the oligonucleotide comprises a total of 24 or fewer interlinked nucleotides.
42. A double-stranded oligonucleotide comprising the oligonucleotide of any one of items 1 to 41 hybridized to a complementary nucleotide.
43. A double-stranded oligonucleotide comprising a passenger strand hybridized to a guide strand comprising a nucleobase sequence comprising at least 6 contiguous nucleobases complementary to an equal-length portion within a NRL target nucleic acid, wherein each of the passenger strand and the guide strand comprises a total of 12 to 50 interlinked nucleotides.
44. The oligonucleotide of item 43, wherein the passenger strand comprises at least one modified nucleobase.
45. The oligonucleotide of item 44, wherein at least one modified nucleobase is 5-methylcytosine.
46. The oligonucleotide of item 43 or 44, wherein at least one modified nucleobase is 7-deazaguanine.
47. The oligonucleotide of any one of items 44 to 46, wherein at least one modified nucleobase is 6-thioguanine.
48. The oligonucleotide of any one of items 43 to 47, wherein the passenger strand comprises at least one modified internucleoside linkage.
49. The oligonucleotide of item 48, wherein the modified internucleoside linkage is a phosphorothioate linkage.
50. The oligonucleotide of item 49, wherein the phosphorothioate linkage is a stereochemically enriched phosphorothioate linkage.
51. The oligonucleotide of any one of items 48 to 59, wherein at least 50% of internucleoside linkages in the passenger strand are each independently the modified internucleoside linkage.
52. The oligonucleotide of item 51, wherein at least 70% of internucleoside linkages in the passenger strand are each independently the modified internucleoside linkage.
53. The oligonucleotide of any one of items 43 to 52, wherein the passenger strand comprises at least one modified sugar nucleoside.
54. The oligonucleotide of item 53, wherein at least one modified sugar nucleoside is a bridged nucleic acid.
55. The oligonucleotide of item 54, wherein the bridged nucleic acid is a locked nucleic acid (LNA), ethylene-bridged nucleic acid (ENA), or cEt nucleic acid.
56. The oligonucleotide of any one of items 53 to 55, wherein at least one modified sugar nucleoside is a 2′-modified sugar nucleoside.
57. The oligonucleotide of item 56, wherein at least one 2′-modified sugar nucleoside comprises a 2′-modification selected from the group consisting of 2′-fluoro, 2′-methoxy, and 2′-methoxyethoxy.
58. The oligonucleotide of any one of items 43 to 57, wherein the passenger strand comprises deoxyribonucleotides.
59. The oligonucleotide of any one of items 43 to 58, wherein the passenger strand comprises ribonucleotides.
60. The oligonucleotide of any one of items 43 to 59, wherein the passenger strand comprises a hydrophobic moiety covalently attached at a 5′-terminus, 3′-terminus, or internucleoside linkage of the passenger strand.
61. The oligonucleotide of any one of items 43 to 60, wherein the guide strand comprises at least one modified nucleobase.
62. The oligonucleotide of item 61, wherein at least one modified nucleobase is 5-methylcytosine.
63. The oligonucleotide of item 61 or 62, wherein at least one modified nucleobase is 7-deazaguanine.
64. The oligonucleotide of any one of items 61 to 63, wherein at least one modified nucleobase is 6-thioguanine.
65. The oligonucleotide of any one of items 43 to 64, wherein the guide strand comprises at least one modified internucleoside linkage.
66. The oligonucleotide of item 65, wherein the modified internucleoside linkage is a phosphorothioate linkage.
67. The oligonucleotide of item 66, wherein the phosphorothioate linkage is a stereochemically enriched phosphorothioate linkage.
68. The oligonucleotide of any one of items 65 to 67, wherein at least 50% of internucleoside linkages in the guide strand are each independently the modified internucleoside linkage.
69. The oligonucleotide of item 68, wherein at least 70% of internucleoside linkages in the guide strand are each independently the modified internucleoside linkage.
70. The oligonucleotide of any one of items 43 to 69, wherein the guide strand comprises at least one modified sugar nucleoside.
71. The oligonucleotide of item 70, wherein at least one modified sugar nucleoside is a bridged nucleic acid.
72. The oligonucleotide of item 71, wherein the bridged nucleic acid is a locked nucleic acid (LNA), ethylene-bridged nucleic acid (ENA), or cEt nucleic acid.
73. The oligonucleotide of any one of items 70 to 72, wherein at least one modified sugar nucleoside is a 2′-modified sugar nucleoside.
74. The oligonucleotide of item 73, wherein at least one 2′-modified sugar nucleoside comprises a 2′-modification selected from the group consisting of 2′-fluoro, 2′-methoxy, and 2′-methoxyethoxy.
75. The oligonucleotide of any one of items 43 to 74, wherein the guide strand comprises deoxyribonucleotides.
76. The oligonucleotide of any one of items 43 to 75, wherein the guide strand comprises ribonucleotides.
77. The oligonucleotide of any one of items 43 to 76, wherein the guide strand comprises a hydrophobic moiety covalently attached at a 5′-terminus, 3′-terminus, or internucleoside linkage of the passenger strand.
78. The oligonucleotide of any one of items 43 to 77, wherein the guide strand comprises a region complementary to a coding sequence within the NRL target nucleic acid.
79. The oligonucleotide of any one of items 43 to 78, wherein the NRL target nucleic acid is NRL transcript 1.
80. The oligonucleotide of any one of items 43 to 78, wherein the NRL target nucleic acid is NRL transcript 2.
81. The oligonucleotide of any one of items 43 to 78, wherein the NRL target nucleic acid is NRL transcript 3.
82. The oligonucleotide of any one of items 43 to 78, wherein the NRL target nucleic acid is NRL transcript 4.
83. The oligonucleotide of any one of items 42 to 76, wherein the guide strand comprises a sequence complementary to a sequence comprising positions 586-605 or 264-283 in NRL transcript 1.
84. The oligonucleotide of any one of items 42 to 76, wherein the guide strand comprises a sequence complementary to a sequence comprising positions 815-834 or 965-984 in NRL transcript 1.
85. The oligonucleotide of any one of items 42 to 83, wherein the hybridized oligonucleotide comprises at least one 3′-overhang.
86. The oligonucleotide of any one of items 42 to 84, wherein the hybridized oligonucleotide is a blunt.
87. The oligonucleotide of any one of items 42 to 84, wherein the hybridized oligonucleotide comprises two 3′-overhangs.
88. A pharmaceutical composition comprising the oligonucleotide of any one of item 1 to 86 and a pharmaceutically acceptable excipient.
89. A method of inhibiting the production of an NRL protein in a cell comprising an NRL gene, the method comprising contacting the cell with the oligonucleotide of any one of items 1 to 86.
90. The method of item 88, wherein the cell is in a subject.
91. The method of item 89, wherein the cell is in the subject's eye.
92. A method of treating a subject in need thereof, the method comprising administering to the subject a therapeutically effective amount of the oligonucleotide of any one of items 1 to 80 or the pharmaceutical composition of item 87.
93. The method of any one of items 89 to 91, wherein the oligonucleotide or pharmaceutical composition is administered intraocularly or topically to the eye of the subject.
94. The method of any one of items 89 to 92, wherein the subject is in need of a treatment for an ocular disease, disorder, or condition associated with a dysfunction of ABCA4, AIPL1, BBS1, BEST1, CEP290, CDH3, CHM, CNGA3, CNGB3, CRB1, GUCY2D, MERTK, MRFP, MYO7A, ND4, NR2E3, PDE6, PRPH2, RD3, RHO, RLBP1, RP1, RPE65, RPGR, RPGRIP1, RS1, or SPATA7 gene.
95. The method of any one of items 89 to 92, wherein the subject is in need of a treatment for retinitis pigmentosa, Stargardt disease, cone-rod dystrophy, Leber congenital amaurosis, Bardet Biedl syndrome, macular dystrophy, dry macular degeneration, geographic atrophy, atrophic age-related macular degeneration (AMD), advanced dry AMD, retinal dystrophy, choroideremia, Usher syndrome type 1, retinoschisis, Leber hereditary optic neuropathy, and achromatopsia.
96. The method of item 94, wherein the subject is in need of a treatment for retinitis pigmentosa.
97. The method of item 95, wherein retinitis pigmentosa is Rho P23H-associated retinitis pigmentosa, PDE6-associated retinitis pigmentosa, MERTK-associated retinitis pigmentosa, BBS1-associated retinitis pigmentosa, Rho-associated retinitis pigmentosa, MRFP-associated retinitis pigmentosa, RLBP1-associated retinitis pigmentosa, RP1-associated retinitis pigmentosa, RPGR-X-linked retinitis pigmentosa, NR2E3-associated retinitis pigmentosa, or SPATA7-associated retinitis pigmentosa.

### Definitions

The term “acyl,” as used herein, represents a chemical substituent of formula —C(O)—R, where R is alkyl, aryl, arylalkyl, cycloalkyl, heterocyclyl, heterocyclyl alkyl, heteroaryl, or heteroaryl alkyl. An optionally substituted acyl is an acyl that is optionally substituted as described herein for each group R.

The term “acyloxy,” as used herein, represents a chemical substituent of formula —OR, where R is acyl. An optionally substituted acyloxy is an acyloxy that is optionally substituted as described herein for acyl.

The term “aliphatic,” as used herein, refers to an acyclic, branched or acyclic, linear hydrocarbon chain, or a monocyclic, bicyclic, tricyclic, or tetracyclic hydrocarbon. Unless specified otherwise, an aliphatic group includes a total of 1 to 60 carbon atoms. An optionally substituted aliphatic is an optionally substituted acyclic aliphatic or an optionally substituted cyclic aliphatic. An optionally substituted acyclic aliphatic is optionally substituted as described herein for alkyl. An optionally substituted cyclic aliphatic is an optionally substituted aromatic aliphatic or an optionally substituted non-aromatic aliphatic. An optionally substituted aromatic aliphatic is optionally substituted as described herein for alkyl. An optionally substituted non-aromatic aliphatic is optionally substituted as described herein for cycloalkyl. In some embodiments, an acyclic aliphatic is alkyl. In certain embodiments, a cyclic aliphatic is aryl. In particular embodiments, a cyclic aliphatic is cycloalkyl.

The term “alkanoyl,” as used herein, represents a chemical substituent of formula —C(O)—R, where R is alkyl. An optionally substituted alkanoyl is an alkanoyl that is optionally substituted as described herein for alkyl.

The term “alkoxy,” as used herein, represents a chemical substituent of formula —OR, where R is a C₁₋₆ alkyl group, unless otherwise specified. An optionally substituted alkoxy is an alkoxy group that is optionally substituted as defined herein for alkyl.

The term “alkyl,” as used herein, refers to an acyclic straight or branched chain saturated hydrocarbon group, which, when unsubstituted, has from 1 to 12 carbons, unless otherwise specified. In certain preferred embodiments, unsubstituted alkyl has from 1 to 6 carbons. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; cyano; ═O; ═S; and ═NR′, where R′ is H, alkyl, aryl, or heterocyclyl. In some embodiments, two substituents combine to form a group -L-CO—R, where L is a bond or optionally substituted C₁₋₁₁ alkylene, and R is hydroxyl or alkoxy. Each of the substituents may itself be unsubstituted or, valency permitting, substituted with unsubstituted substituent(s) defined herein for each respective group.

The term “alkylene,” as used herein, represents a divalent substituent that is an alkyl having one hydrogen atom replaced with a valency. An optionally substituted alkylene is an alkylene that is optionally substituted as described herein for alkyl.

The term “altmer,” as used herein, refers to an oligonucleotide having a pattern of structural features characterized by internucleoside linkages, in which no two consecutive internucleoside linkages have the same structural feature. In some embodiments, an altmer is designed such that it includes a repeating pattern. In some embodiments, an altmer is designed such that it does not include a repeating pattern. In instances, where the “same structural feature” refers to the stereochemical configuration of the internucleoside linkages, the altmer is a “stereoaltmer.”

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of: alkyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; and cyano. Each of the substituents may itself be unsubstituted or substituted with unsubstituted substituent(s) defined herein for each respective group.

The term “aryl alkyl,” as used herein, represents an alkyl group substituted with an aryl group. The aryl and alkyl portions may be optionally substituted as the individual groups as described herein.

The term “arylene,” as used herein, represents a divalent substituent that is an aryl having one hydrogen atom replaced with a valency. An optionally substituted arylene is an arylene that is optionally substituted as described herein for aryl.

The term “aryloxy,” as used herein, represents a group —OR, where R is aryl. Aryloxy may be an optionally substituted aryloxy. An optionally substituted aryloxy is aryloxy that is optionally substituted as described herein for aryl.

The term “bicyclic sugar moiety,” as used herein, represents a modified sugar moiety including two fused rings. In certain embodiments, the bicyclic sugar moiety includes a furanosyl ring.

The term “blockmer,” as used herein, refers to an oligonucleotide strand having a pattern of structural features characterized by the presence of at least two consecutive internucleoside linkages with the same structural feature. By same structural feature is meant the same stereochemistry at the internucleoside linkage phosphorus or the same modification at the linkage phosphorus. The two or more consecutive internucleoside linkages with the same structure feature are referred to as a “block.” In instances, where the “same structural feature” refers to the stereochemical configuration of the internucleoside linkages, the blockmer is a “stereoblockmer.”

The expression “C_{x-y},” as used herein, indicates that the group, the name of which immediately follows the expression, when unsubstituted, contains a total of from x to y carbon atoms. If the group is a composite group (e.g., aryl alkyl), C_{x-y} indicates that the portion, the name of which immediately follows the expression, when unsubstituted, contains a total of from x to y carbon atoms. For example, (C₆₋₁₀-aryl)-C₁₋₆-alkyl is a group, in which the aryl portion, when unsubstituted, contains a total of from 6 to 10 carbon atoms, and the alkyl portion, when unsubstituted, contains a total of from 1 to 6 carbon atoms.

The term “complementary,” as used herein in reference to a nucleobase sequence, refers to the nucleobase sequence having a pattern of contiguous nucleobases that permits an oligonucleotide having the nucleobase sequence to hybridize to another oligonucleotide or nucleic acid to form a duplex structure under physiological conditions. Complementary sequences include Watson-Crick base pairs formed from natural and/or modified nucleobases. Complementary sequences can also include non-Watson-Crick base pairs, such as wobble base pairs (guanosine-uracil, hypoxanthine-uracil, hypoxanthine-adenine, and hypoxanthine-cytosine) and Hoogsteen base pairs.

The term “contiguous,” as used herein in the context of an oligonucleotide, refers to nucleosides, nucleobases, sugar moieties, or internucleoside linkages that are immediately adjacent to each other. For example, “contiguous nucleobases” means nucleobases that are immediately adjacent to each other in a sequence.

The term “cycloalkyl,” as used herein, refers to a cyclic alkyl group having from three to ten carbons (e.g., a C₃-C₁₀ cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be unsubstituted or substituted (e.g., optionally substituted cycloalkyl) with one, two, three, four, or five substituents independently selected from the group consisting of: alkyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; cyano; ═O; ═S; ═NR′, where R′ is H, alkyl, aryl, or heterocyclyl. Each of the substituents may itself be unsubstituted or substituted with unsubstituted substituent(s) defined herein for each respective group.

The term “cycloalkylene,” as used herein, represents a divalent substituent that is a cycloalkyl having one hydrogen atom replaced with a valency. An optionally substituted cycloalkylene is a cycloalkylene that is optionally substituted as described herein for cycloalkyl.

The term “cycloalkoxy,” as used herein, represents a group —OR, where R is cycloalkyl. Cycloalkoxy may be an optionally substituted cycloalkoxy. An optionally substituted cycloalkoxy is cycloalkoxy that is optionally substituted as described herein for cycloalkyl.

The term “duplex,” as used herein, represents two oligonucleotides that are paired through hybridization of complementary nucleobases.

The term “gapmer,” as used herein, refers to an oligonucleotide having an RNase H recruiting region (gap) flanked by a 5′ wing and 3′ wing, each of the wings including at least one affinity enhancing nucleoside (e.g., 1, 2, 3, or 4 affinity enhancing nucleosides).

The term “halo,” as used herein, represents a halogen selected from bromine, chlorine, iodine, and fluorine.

The term “headmer,” as used herein, refers to an oligonucleotide having an RNase H recruiting region (gap) flanked by a 5′ wing including at least one affinity enhancing nucleoside (e.g., 1, 2, 3, or 4 affinity enhancing nucleosides).

The term “heteroalkyl,” as used herein refers to an alkyl group interrupted one or more times by one or two heteroatoms each time. Each heteroatom is, independently, O, N, or S. None of the heteroalkyl groups includes two contiguous oxygen atoms. The heteroalkyl group may be unsubstituted or substituted (e.g., optionally substituted heteroalkyl). When heteroalkyl is substituted and the substituent is bonded to the heteroatom, the substituent is selected according to the nature and valency of the heteroatom. Thus, the substituent bonded to the heteroatom, valency permitting, is selected from the group consisting of ═O, —N(RN²)₂, —SO₂ORN³, —SO₂RN², —SORN³, —COOR^{N3}, an N protecting group, alkyl, aryl, cycloalkyl, heterocyclyl, or cyano, where each RN² is independently H, alkyl, cycloalkyl, aryl, or heterocyclyl, and each RN³ is independently alkyl, cycloalkyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or I. It is understood that carbon atoms are found at the termini of a heteroalkyl group. In some embodiments, heteroalkyl is PEG

The term “heteroalkylene,” as used herein, represents a divalent substituent that is a heteroalkyl having one hydrogen atom replaced with a valency. An optionally substituted heteroalkylene is a heteroalkylene that is optionally substituted as described herein for heteroalkyl.

The term “heteroaryl,” as used herein, represents a monocyclic 5-, 6-, 7-, or 8-membered ring system, or a fused or bridging bicyclic, tricyclic, or tetracyclic ring system; the ring system contains one, two, three, or four heteroatoms independently selected from the group consisting of nitrogen, oxygen, and sulfur; and at least one of the rings is an aromatic ring. Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl, tetrazolyl, dihydroindolyl, tetrahydroquinolyl, tetrahydroisoquinolyl, etc. The term bicyclic, tricyclic, and tetracyclic heteroaryls include at least one ring having at least one heteroatom as described above and at least one aromatic ring. For example, a ring having at least one heteroatom may be fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heteroaryls include 1,2,3,5,8,8a-hexahydroindolizine; 2,3-dihydrobenzofuran; 2,3-dihydroindole; and 2,3-dihydrobenzothiophene. Heteroaryl may be optionally substituted with one, two, three, four, or five substituents independently selected from the group consisting of: alkyl; alkoxy; acyloxy; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thiol; cyano; ═O; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl. Each of the substituents may itself be unsubstituted or substituted with unsubstituted substituent(s) defined herein for each respective group.

The term “heteroaryloxy,” as used herein, refers to a structure —OR, in which R is heteroaryl. Heteroaryloxy can be optionally substituted as defined for heteroaryl.

The term “heterocyclyl,” as used herein, represents a monocyclic, bicyclic, tricyclic, or tetracyclic ring system having fused or bridging 4-, 5-, 6-, 7-, or 8-membered rings, unless otherwise specified, the ring system containing one, two, three, or four heteroatoms independently selected from the group consisting of nitrogen, oxygen, and sulfur. Heterocyclyl may be aromatic or non-aromatic. An aromatic heterocyclyl is heteroaryl as described herein. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups have a carbon count of 1 to 16 carbon atoms unless otherwise specified. Certain heterocyclyl groups may have a carbon count up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, pyranyl, dihydropyranyl, dithiazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., a cyclohexane ring, a cyclohexene ring, a cyclopentane ring, a cyclopentene ring, or another heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkoxy; acyloxy; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thiol; cyano; ═O; ═S; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl.

The term “heterocyclyl alkyl,” as used herein, represents an alkyl group substituted with a heterocyclyl group. The heterocyclyl and alkyl portions of an optionally substituted heterocyclyl alkyl are optionally substituted as described for heterocyclyl and alkyl, respectively.

The term “heterocyclylene,” as used herein, represents a divalent substituent that is a heterocyclyl having one hydrogen atom replaced with a valency. An optionally substituted heterocyclylene is a heterocyclylene that is optionally substituted as described herein for heterocyclyl.

The term “heterocyclyloxy,” as used herein, refers to a structure —OR, in which R is heterocyclyl. Heterocyclyloxy can be optionally substituted as described for heterocyclyl.

The terms “hydroxyl” and “hydroxy,” as used interchangeably herein, represent —OH.

The term “hydrophobic moiety,” as used herein, represents a monovalent group covalently linked to an oligonucleotide backbone, where the monovalent group is a bile acid (e.g., cholic acid, taurocholic acid, deoxycholic acid, oleyl lithocholic acid, or oleoyl cholenic acid), glycolipid, phospholipid, sphingolipid, isoprenoid, vitamin, saturated fatty acid, unsaturated fatty acid, fatty acid ester, triglyceride, pyrene, porphyrine, texaphyrine, adamantine, acridine, biotin, coumarin, fluorescein, rhodamine, Texas-Red, digoxygenin, dimethoxytrityl, t-butydimethylsilyl, t-butyldiphenylsilyl, cyanine dye (e.g., Cy3 or Cy5), Hoechst 33258 dye, psoralen, or ibuprofen. Non-limiting examples of the monovalent group include ergosterol, stigmasterol, β-sitosterol, campesterol, fucosterol, saringosterol, avenasterol, coprostanol, cholesterol, vitamin A, vitamin D, vitamin E, cardiolipin, and carotenoids. The linker connecting the monovalent group to the oligonucleotide may be an optionally substituted C₁₋₆₀ aliphatic (e.g., optionally substituted C₁₋₆₀ alkylene) or an optionally substituted C₂₋₆₀ heteroaliphatic (e.g., optionally substituted C₂₋₆₀ heteroalkylene), where the linker may be optionally interrupted with one, two, or three instances independently selected from the group consisting of an optionally substituted arylene, optionally substituted heterocyclylene, and optionally substituted cycloalkylene. The linker may be bonded to an oligonucleotide through, e.g., an oxygen atom attached to a 5′-terminal carbon atom, a 3′-terminal carbon atom, a 5′-terminal phosphate or phosphorothioate, a 3′-terminal phosphate or phosphorothioate, or an internucleoside linkage.

The term “internucleoside linkage,” as used herein, represents a group or bond that forms a covalent linkage between adjacent nucleosides in an oligonucleotide. An internucleoside linkage is an unmodified internucleoside linkage or a modified internucleoside linkage. An “unmodified internucleoside linkage” is a phosphate (—O—P(O)(OH)—O—) internucleoside linkage (“phosphate phosphodiester”). A “modified internucleoside linkage” is an internucleoside linkage other than a phosphate phosphodiester. The two main classes of modified internucleoside linkages are defined by the presence or absence of a phosphorus atom. Non-limiting examples of phosphorus-containing internucleoside linkages include phosphodiester linkages, phosphotriester linkages, phosphorothioate diester linkages, phosphorothioate triester linkages, morpholino internucleoside linkages, methylphosphonates, and phosphoramidate. Non-limiting examples of non-phosphorus internucleoside linkages include methylenemethylimino (—CH₂—N(CH₃)—O—CH₂—), thiodiester (—O—C(O)—S—), thionocarbamate (—O—C(O)(NH)—S—), siloxane (—O—Si(H)₂—O—), and N,N′-dimethylhydrazine (—CH₂—N(CH₃)—N(CH₃)—). Phosphorothioate linkages are phosphodiester linkages and phosphotriester linkages in which one of the non-bridging oxygen atoms is replaced with a sulfur atom. In some embodiments, an internucleoside linkage is a group of the following structure:

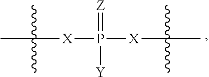

where

Z is O, S, or Se;

Y is —X-L-R¹;

each X is independently —O, S, N(-L-R¹)—, or L;

each L is independently a covalent bond or a linker (e.g., optionally substituted C₁₋₆₀ aliphatic linker or optionally substituted C₂₋₆₀ heteroaliphatic linker);

each R¹ is independently hydrogen, —S—S—R², —O—CO—R², —S—CO—R², optionally substituted C₁₋₉ heterocyclyl, or a hydrophobic moiety; and

each R² is independently optionally substituted C₁₋₁₀ alkyl, optionally substituted C₂₋₁₀ heteroalkyl, optionally substituted C₆₋₁₀ aryl, optionally substituted C₆₋₁₀ aryl C₁₋₆ alkyl, optionally substituted C₁₋₉ heterocyclyl, or optionally substituted C₁₋₉ heterocyclyl C₁₋₆ alkyl.

When L is a covalent bond, R¹ is hydrogen, Z is oxygen, and all X groups are —O—, the internucleoside group is known as a phosphate phosphodiester. When L is a covalent bond, R¹ is hydrogen, Z is sulfur, and all X groups are —O—, the internucleoside group is known as a phosphorothioate diester. When Z is oxygen, all X groups are —O—, and either (1) L is a linker or (2) R¹ is not a hydrogen, the internucleoside group is known as a phosphotriester. When Z is sulfur, all X groups are —O—, and either (1) L is a linker or (2) R¹ is not a hydrogen, the internucleoside group is known as a phosphorothioate triester. Non-limiting examples of phosphorothioate triester linkages and phosphotriester linkages are described in US 2017/0037399, the disclosure of which is incorporated herein by reference.

The term “morpholino,” as used herein in reference to a class of oligonucleotides, represents an oligomer of at least 10 morpholino monomer units interconnected by morpholino internucleoside linkages. A morpholino includes a 5′ group and a 3′ group. For example, a morpholino may be of the following structure:

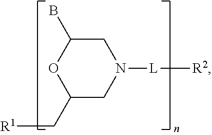

where

n is an integer of at least 10 (e.g., 12 to 30) indicating the number of morpholino units;

each B is independently a nucleobase;

R¹ is a 5′ group;

R² is a 3′ group; and

L is (i) a morpholino internucleoside linkage or, (ii) if L is attached to R², a covalent bond. A 5′ group in morpholino may be, e.g., hydroxyl, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer. A 3′ group in morpholino may be, e.g., hydrogen, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer.

The term “morpholino internucleoside linkage,” as used herein, represents a divalent group of the following structure:

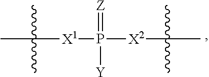

where

Z is O or S;

X¹ is a bond, —CH₂—, or —O—;

X² is a bond, —CH₂—O—, or −O—; and

Y is —NR₂, where each R is independently Cis alkyl (e.g., methyl), or both R combine together with the nitrogen atom to which they are attached to form a C₂₋₉ heterocyclyl (e.g., N-piperazinyl); provided that both X¹ and X² are not simultaneously a bond.

The term “NRL,” as used herein, represents refers to a ribonucleic acid (e.g., pre-mRNA or mRNA) that encodes the protein Neural Retina Leucine Zipper in humans. An exemplary genomic DNA sequence of a human NRL gene is given by SEQ ID NO. 1 (NCBI Reference Sequence: NG_011697.2). One of skill in the art will recognize that a pre-mRNA is produced from the genomic DNA in accordance with the central dogma; pre-mRNA is then spliced to produce transcripts, e.g., NRL transcript 1, NRL transcript 2, NRL transcript 3, or NRL transcript 4. Exemplary mRNA sequences of a human NRL gene are given by SEQ ID NOs. 2, 3, 4, and 5 (NCBI Reference Sequences: NM_006177.4, NM_001354768.1, NM_001354769.1, and NM_001354770.1). SEQ ID NO. 2 corresponds to NRL transcript 1. SEQ ID NO. 3 corresponds to NRL transcript 2. SEQ ID NO. 4 corresponds to NRL transcript 3. SEQ ID NO. 5 corresponds to NRL transcript 4. SEQ ID NOs. 2, 3, 4, and 5 are based on NCBI Reference Sequences for NRL transcripts 1, 2, 3, and 4, which are provided as RNA sequences with thymidines in the NCBI Reference Sequences. One of skill in the art will recognize that an RNA sequence typically includes uridines instead of thymidines. Accordingly, target RNA sequences may include one or more uridines instead of thymidines without affecting the sequence of an oligonucleotide of the invention.

The term “nucleobase,” as used herein, represents a nitrogen-containing heterocyclic ring found at the 1′ position of the ribofuranose/2′-deoxyribofuranose of a nucleoside. Nucleobases are unmodified or modified. As used herein, “unmodified” or “natural” nucleobases include the purine bases adenine (A) and guanine (G), and the pyrimidine bases thymine (T), cytosine (C), and uracil (U). Modified nucleobases include 5-substituted pyrimidines, 6-azapyrimidines, alkyl or alkynyl substituted pyrimidines, alkyl substituted purines, and N-2, N-6 and 0-6 substituted purines, as well as synthetic and natural nucleobases, e.g., 5-methylcytosine, 5-hydroxymethyl cytosine, xanthine, hypoxanthine, 2-aminoadenine, 6-alkyl (e.g., 6-methyl) adenine and guanine, 2-alkyl (e.g., 2-propyl) adenine and guanine, 2-thiouracil, 2-thiothymine, 2-thiocytosine, 5-halouracil, 5-halocytosine, 5-propynyl uracil, 5-propynyl cytosine, 5-trifluoromethyl uracil, 5-trifluoromethyl cytosine, 7-methyl guanine, 7-methyl adenine, 8-azaguanine, 8-azaadenine, 7-deazaguanine, 7-deazaadenine, 3-deazaguanine, 3-deazaadenine. Certain nucleobases are particularly useful for increasing the binding affinity of nucleic acids, e g., 5-substituted pyrimidines; 6-azapyrimidines; N2-, N6-, and/or O6-substituted purines. Nucleic acid duplex stability can be enhanced using, e.g., 5-methylcytosine. Non-limiting examples of nucleobases include: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH3) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example, 7-deazaadenine, 7-deazaguanine, 2-aminopyridine, or 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

The term “nucleoside,” as used herein, represents sugar-nucleobase compounds and groups known in the art, as well as modified or unmodified 2′-deoxyribofuranose-nucleobase compounds and groups known in the art. The sugar may be ribofuranose. The sugar may be modified or unmodified. An unmodified ribofuranose-nucleobase is ribofuranose having an anomeric carbon bond to an unmodified nucleobase. Unmodified ribofuranose-nucleobases are adenosine, cytidine, guanosine, and uridine. Unmodified 2′-deoxyribofuranose-nucleobase compounds are 2′-deoxyadenosine, 2′-deoxycytidine, 2′-deoxyguanosine, and thymidine. The modified compounds and groups include one or more modifications selected from the group consisting of nucleobase modifications and sugar modifications described herein. A nucleobase modification is a replacement of an unmodified nucleobase with a modified nucleobase. A sugar modification may be, e.g., a 2′-substitution, locking, carbocyclization, or unlocking. A 2′-substitution is a replacement of 2′-hydroxyl in ribofuranose with 2′-fluoro, 2′-methoxy, or 2′-(2-methoxy)ethoxy. Alternatively, a 2′-substitution may be a 2′-(ara) substitution, which corresponds to the following structure:

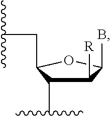

where B is a nucleobase, and R is a 2′-(ara) substituent (e.g., fluoro). 2′-(ara) substituents are known in the art and can be same as other 2′-substituents described herein. In some embodiments, 2′-(ara) substituent is a 2′-(ara)-F substituent (R is fluoro). A locking modification is an incorporation of a bridge between 4′-carbon atom and 2′-carbon atom of ribofuranose. Nucleosides having a locking modification are known in the art as bridged nucleic acids, e.g., locked nucleic acids (LNA), ethylene-bridged nucleic acids (ENA), and cEt nucleic acids. The bridged nucleic acids are typically used as affinity enhancing nucleosides.

The term “nucleotide,” as used herein, represents a nucleoside bonded to an internucleoside linkage or a monovalent group of the following structure —X¹—P(X²)(R¹)₂, where X¹ is O, S, or NH, and X² is absent, ═O, or ═S, and each R¹ is independently —OH, —N(R²)₂, or —O—CH₂CH₂CN, where each R² is independently an optionally substituted alkyl, or both R² groups, together with the nitrogen atom to which they are attached, combine to form an optionally substituted heterocyclyl.

The term “oligonucleotide,” as used herein, represents a structure containing 10 or more contiguous nucleosides covalently bound together by internucleoside linkages. An oligonucleotide includes a 5′ end and a 3′ end. The 5′ end of an oligonucleotide may be, e.g., hydroxyl, a hydrophobic moiety, 5′ cap, phosphate, diphosphate, triphosphate, phosphorothioate, diphosphorothioate, triphosphorothioate, phosphorodithioate, diphosphrodithioate, triphosphorodithioate, phosphonate, phosphoramidate, a cell penetrating peptide, an endosomal escape moiety, or a neutral organic polymer. The 3′ end of an oligonucleotide may be, e.g., hydroxyl, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer (e.g., polyethylene glycol). An oligonucleotide having a 5′-hydroxyl or 5′-phosphate has an unmodified 5′ terminus. An oligonucleotide having a 5′ terminus other than 5′-hydroxyl or 5′-phosphate has a modified 5′ terminus. An oligonucleotide having a 3′-hydroxyl or 3′-phosphate has an unmodified 3′ terminus. An oligonucleotide having a 3′ terminus other than 3′-hydroxyl or 3′-phosphate has a modified 3′ terminus. Oligonucleotides can be in double- or single-stranded form. Double-stranded oligonucleotide molecules can optionally include one or more single-stranded segments (e.g., overhangs).

The term “oxo,” as used herein, represents a divalent oxygen atom (e.g., the structure of oxo may be shown as ═O).

The term “pharmaceutically acceptable,” as used herein, refers to those compounds, materials, compositions, and/or dosage forms, which are suitable for contact with the tissues of an individual (e.g., a human), without excessive toxicity, irritation, allergic response and other problem complications commensurate with a reasonable benefit/risk ratio.

The term “pharmaceutical composition,” as used herein, represents a composition containing an oligonucleotide described herein, formulated with a pharmaceutically acceptable excipient, and manufactured or sold with the approval of a governmental regulatory agency as part of a therapeutic regimen for the treatment of disease in a subject.

The term “protecting group,” as used herein, represents a group intended to protect a functional group (e.g., a hydroxyl, an amino, or a carbonyl) from participating in one or more undesirable reactions during chemical synthesis. The term “O-protecting group,” as used herein, represents a group intended to protect an oxygen containing (e.g., phenol, hydroxyl or carbonyl) group from participating in one or more undesirable reactions during chemical synthesis. The term “N-protecting group,” as used herein, represents a group intended to protect a nitrogen containing (e.g., an amino or hydrazine) group from participating in one or more undesirable reactions during chemical synthesis. Commonly used O- and N-protecting groups are disclosed in Greene, “Protective Groups in Organic Synthesis,” 3^{rd} Edition (John Wiley & Sons, New York, 1999), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and arylalkyl ethers (e.g., trityl; methylthiomethyl; methoxymethyl; benzyloxymethyl; siloxymethyl; 2,2,2-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydroxy carbonyl, t-butyloxycarbonyl, diisopropylmethoxycarbonyl, isopropoxycarbonyl, ethoxycarbonyl, methoxycarbonyl, allyloxycarbonyl, 2,2,2-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, arylalkyl groups such as benzyl, triphenylmethyl, benzyloxymethyl, and the like and silyl groups such as trimethylsilyl, and the like.

The term “shRNA,” as used herein, refers to a double-stranded oligonucleotide of the invention having a passenger strand and a guide strand, where the passenger strand and the guide strand are covalently linked by a linker excisable through the action of the Dicer enzyme.

The term “siRNA,” as used herein, refers to a double-stranded oligonucleotide of the invention having a passenger strand and a guide strand, where the passenger strand and the guide strand are not covalently linked to each other.

The term “skipmer,” as used herein, refers a gapmer, in which alternating internucleoside linkages are phosphate phosphodiester linkages and intervening internucleoside linkages are modified internucleoside linkages.

The term “stereochemically enriched,” as used herein, refers to a local stereochemical preference for one enantiomer of the recited group over the opposite enantiomer of the same group. Thus, an oligonucleotide containing a stereochemically enriched internucleoside linkage is an oligonucleotide, in which a phosphorothioate of predetermined stereochemistry is present in preference to a phosphorothioate of stereochemistry that is opposite of the predetermined stereochemistry. This preference can be expressed numerically using a diastereomeric ratio for the phosphorothioate of the predetermined stereochemistry. The diastereomeric ratio for the phosphorothioate of the predetermined stereochemistry is the molar ratio of the diastereomers having the identified phosphorothioate with the predetermined stereochemistry relative to the diastereomers having the identified phosphorothioate with the stereochemistry that is opposite of the predetermined stereochemistry. The diastereomeric ratio for the phosphorothioate of the predetermined stereochemistry may be greater than or equal to 1.1 (e.g., greater than or equal to 4, greater than or equal to 9, greater than or equal to 19, or greater than or equal to 39).

The term “subject,” as used herein, represents a human or non-human animal (e.g., a mammal) that is suffering from, or is at risk of, disease, disorder, or condition, as determined by a qualified professional (e.g., a doctor or a nurse practitioner) with or without known in the art laboratory test(s) of sample(s) from the subject. Non-limiting examples of diseases, disorders, and conditions include retinitis pigmentosa (e.g., Rho P23H-associated retinitis pigmentosa, PDE6-associated retinitis pigmentosa, MERTK-associated retinitis pigmentosa, BBS1-associated retinitis pigmentosa, Rho-associated retinitis pigmentosa, MRFP-associated retinitis pigmentosa, RLBP1-associated retinitis pigmentosa, RP1-associated retinitis pigmentosa, RPGR-X-linked retinitis pigmentosa, NR2E3-associated retinitis pigmentosa, or SPATA7-associated retinitis pigmentosa), Stargardt disease (e.g., ABCA4-associated Stargardt disease), cone-rod dystrophy (e.g., AIPL1-associated cone-rod dystrophy or RGRIP1-associated cone-rod dystrophy), Leber congenital amaurosis (e.g., AIPL1-associated Leber congenital amaurosis, GUCY2D-associated Leber congenital amaurosis, RD3-associated Leber congenital amaurosis, RPE65-associated Leber congenital amaurosis, or SPATA7-associated Leber congenital amaurosis), Bardet Biedl syndrome (e.g., BBS1-associated Bardet Biedl syndrome), macular dystrophy (e.g., BEST1-associated macular dystrophy), dry macular degeneration, geographic atrophy, atrophic age-related macular degeneration (AMD), advanced dry AMD, retinal dystrophy (e.g., CEP290-associated retinal dystrophy, CDH3-associated retinal dystrophy, CRB1-associated retinal dystrophy, or PRPH2-associated retinal dystrophy), choroideremia (e.g., CHM-associated choroideremia), Usher syndrome type 1 (e.g., MYO7A-associated Usher syndrome), retinoschisis (e.g., RS1-X-linked retinoschisis), Leber hereditary optic neuropathy (e.g., ND4-associated Lebe'rs hereditary optic neuropathy), and achromatopsia (e.g., CNGA3-associated achromatopsia or CNGB3-associated achromatopsia). Non-limiting examples of diseases, disorders, and conditions include ocular diseases, disorders, and conditions associated with a dysfunction of ABCA4, AIPL1, BBS1, BEST1, CEP290, CDH3, CHM, CNGA3, CNGB3, CRB1, GUCY2D, MERTK, MRFP, MYO7A, ND4, NR2E3, PDE6, PRPH2, RD3, RHO, RLBP1, RP1, RPE65, RPGR, RPGRIP1, RS1, or SPATA7 gene.

A “sugar” or “sugar moiety,” includes naturally occurring sugars having a furanose ring or a structure that is capable of replacing the furanose ring of a nucleoside. Sugars included in the nucleosides of the invention may be non-furanose (or 4′-substituted furanose) rings or ring systems or open systems. Such structures include simple changes relative to the natural furanose ring (e.g., a six-membered ring). Alternative sugars may also include sugar surrogates wherein the furanose ring has been replaced with another ring system such as, e.g., a morpholino or hexitol ring system. Non-limiting examples of sugar moieties useful that may be included in the oligonucleotides of the invention include β-D-ribose, β-D-2′-deoxyribose, substituted sugars (e.g., 2′, 5′, and bis substituted sugars), 4′-S-sugars (e.g., 4′-S-ribose, 4′-S-2′-deoxyribose, and 4′-S-2′-substituted ribose), bicyclic sugar moieties (e.g., the 2′-O—CH₂-4′ or 2′-O—(CH₂)₂-4′ bridged ribose derived bicyclic sugars) and sugar surrogates (when the ribose ring has been replaced with a morpholino or a hexitol ring system).

The term “tailmer,” as used herein, refers to an oligonucleotide having an RNase H recruiting region (gap) flanked by a 3′ wing including at least one affinity enhancing nucleoside (e.g., 1, 2, 3, or 4 affinity enhancing nucleosides).

“Treatment” and “treating,” as used herein, refer to the medical management of a subject with the intent to improve, ameliorate, stabilize, or prevent a disease, disorder, or condition (e.g., retinitis pigmentosa). This term includes active treatment (treatment directed to improve retinitis pigmentosa); causal treatment (treatment directed to the cause of associated retinitis pigmentosa); palliative treatment (treatment designed for the relief of symptoms of retinitis pigmentosa); preventative treatment (treatment directed to minimizing or partially or completely inhibiting the development of retinitis pigmentosa); and supportive treatment (treatment employed to supplement another therapy).

The term “unimer,” as used herein, refers to an oligonucleotide having a pattern of structural features characterized by all of the internucleoside linkages having the same structural feature. By same structural feature is meant the same stereochemistry at the internucleoside linkage phosphorus or the same modification at the linkage phosphorus. In instances, where the “same structural feature” refers to the stereochemical configuration of the internucleoside linkages, the unimer is a “stereounimer.”

Enumeration of positions within oligonucleotides and nucleic acids, as used herein and unless specified otherwise, starts with the 5′-terminal nucleoside as 1 and proceeds in the 3′-direction.

The compounds described herein, unless otherwise noted, encompass isotopically enriched compounds (e.g., deuterated compounds), tautomers, and all stereoisomers and conformers (e.g. enantiomers, diastereomers, E/Z isomers, atropisomers, etc.), as well as racemates thereof and mixtures of different proportions of enantiomers or diastereomers, or mixtures of any of the foregoing forms as well as salts (e.g., pharmaceutically acceptable salts).

### DETAILED DESCRIPTION

In general, the invention provides oligonucleotides that may be used in the treatment of ocular degeneration disorders (e.g., a retinal degeneration disorder; e.g., retinitis pigmentosa (e.g., Rho P23H-associated retinitis pigmentosa, PDE6-associated retinitis pigmentosa, MERTK-associated retinitis pigmentosa, BBS1-associated retinitis pigmentosa, Rho-associated retinitis pigmentosa, MRFP-associated retinitis pigmentosa, RLBP1-associated retinitis pigmentosa, RP1-associated retinitis pigmentosa, RPGR-X-linked retinitis pigmentosa, NR2E3-associated retinitis pigmentosa, or SPATA7-associated retinitis pigmentosa), Stargardt disease (e.g., ABCA4-associated Stargardt disease), cone-rod dystrophy (e.g., AIPL1-associated cone-rod dystrophy or RGRIP1-associated cone-rod dystrophy), Leber congenital amaurosis (e.g., AIPL1-associated Leber congenital amaurosis, GUCY2D-associated Leber congenital amaurosis, RD3-associated Leber congenital amaurosis, RPE65-associated Leber congenital amaurosis, or SPATA7-associated Leber congenital amaurosis), Bardet Biedl syndrome (e.g., BBS1-associated Bardet Biedl syndrome), macular dystrophy (e.g., BEST1-associated macular dystrophy), dry macular degeneration, geographic atrophy, atrophic age-related macular degeneration (AMD), advanced dry AMD, retinal dystrophy (e.g., CEP290-associated retinal dystrophy, CDH3-associated retinal dystrophy, CRB1-associated retinal dystrophy, or PRPH2-associated retinal dystrophy), choroideremia (e.g., CHM-associated choroideremia), Usher syndrome type 1 (e.g., MYO7A-associated Usher syndrome), retinoschisis (e.g., RS1-X-linked retinoschisis), Leber hereditary optic neuropathy (e.g., ND4-associated Lebe'rs hereditary optic neuropathy), and achromatopsia (e.g., CNGA3-associated achromatopsia or CNGB3-associated achromatopsia)). Without wishing to be bound by theory, reduction of the expression of NRL in photoreceptor cells can prevent loss of photoreceptor cells, thereby treating an ocular degeneration disorder (e.g., a retinal degeneration disorder).

The invention provides two approaches to reducing expression of NRL in cells: an antisense approach and an RNAi approach as described herein. Typically, antisense and RNAi activities may be observed directly or indirectly. Observation or detection of an antisense or RNAi activity involves observation or detection of a change in an amount of a target nucleic acid or protein encoded by such target nucleic acid, a change in the ratio of splice variants of a nucleic acid or protein, and/or a phenotypic change in a cell or animal.

### I. Antisense

In one approach, the invention provides a single-stranded oligonucleotide having a nucleobase sequence with at least 6 contiguous nucleobases complementary to an equal-length portion within an NRL target nucleic acid. This approach is typically referred to as an antisense approach, and the corresponding oligonucleotides of the invention are referred to as antisense oligonucleotides (ASO). Without wishing to be bound by theory, this approach involves hybridization of an oligonucleotide of the invention to a target NRL nucleic acid (e.g., NRL pre-mRNA, NRL transcript 1, NRL transcript 2, NRL transcript 3, or NRL transcript 4), followed by ribonuclease H (RNase H) mediated cleavage of the target NRL nucleic acid. Alternatively and without wishing to be bound by theory, this approach involves hybridization of an oligonucleotide of the invention to a target NRL nucleic acid (e.g., NRL pre-mRNA, NRL transcript 1, NRL transcript 2, NRL transcript 3, or NRL transcript 4), thereby sterically blocking the target NRL nucleic acid from binding cellular post-transcription modification or translation machinery and thus preventing the translation of the target NRL nucleic acid translation. In some embodiments, the single-stranded oligonucleotide may be delivered to a patient as a double stranded oligonucleotide, where the oligonucleotide of the invention is hybridized to another oligonucleotide (e.g., an oligonucleotide having a total of 12 to 30 nucleotides).

An antisense oligonucleotide of the invention (e.g., a single-stranded oligonucleotide of the invention) includes a nucleobase sequence having at least 6 (e.g., at least 7, 8, 9, 10, 11, 12, 13, 14, 15, 16, 17, 18, 19, or 20) contiguous nucleobases complementary to an equal-length portion within an NRL target nucleic acid (e.g., NRL pre-mRNA, NRL transcript 1, NRL transcript 2, NRL transcript 3, or NRL transcript 4). The equal-length portion may be disposed within the sequence from position 547 to position 1260 in NRL transcript 1. The equal-length portion may be disposed within the sequence from position 354 to position 753 in NRL transcript 1. The equal-length portion may be disposed within the sequence from position 569 to position 634 in NRL transcript 1. The equal-length portion may be disposed within the sequence from position 807 to position 866 in NRL transcript 1. The equal-length portion may be disposed within the sequence from position 1149 to position 1260 in NRL transcript 1. The equal-length portion may be disposed within the sequence from position 888 to position 911 in NRL transcript 1. The equal-length portion may include positions 642-645, 766-769, or 1127-1130 in NRL transcript 1. The equal-length portion may include positions 892-895, 974-977, 1175-1178, or 1235-1238 in NRL transcript 1. The equal-length portion may include positions 721-724 in NRL transcript 1. The equal-length portion may include positions 904-907 in NRL transcript 1. The equal-length portion may include positions 825-828, 933-936, or 1031-1034 in NRL transcript 1.

An antisense oligonucleotide of the invention (e.g., a single-stranded oligonucleotide of the invention) may be a gapmer, headmer, or tailmer. Gapmers are oligonucleotides having an RNase H recruiting region (gap) flanked by a 5′ wing and 3′ wing, each of the wings including at least one affinity enhancing nucleoside (e.g., 1, 2, 3, or 4 affinity enhancing nucleosides). Headmers are oligonucleotides having an RNase H recruiting region (gap) flanked by a 5′ wing including at least one affinity enhancing nucleoside (e.g., 1, 2, 3, or 4 affinity enhancing nucleosides). Tailmers are oligonucleotides having an RNase H recruiting region (gap) flanked by a 3′ wing including at least one affinity enhancing nucleoside (e.g., 1, 2, 3, or 4 affinity enhancing nucleosides). In certain embodiments, each wing includes 1-5 nucleosides. In some embodiments, each nucleoside of each wing is a modified nucleoside. In particular embodiments, the gap includes 7-12 nucleosides. Typically, the gap region includes a plurality of contiguous, unmodified deoxyribonucleotides. For example, all nucleotides in the gap region are unmodified deoxyribonucleotides (2′-deoxyribofuranose-based nucleotides). In some embodiments, an antisense oligonucleotide of the invention (e.g., a single-stranded oligonucleotide of the invention) is a gapmer.

The 5′-wing may consists of, e.g., 1 to 8 nucleosides. The 5′-wing may consist of, e.g., 1 to 7 nucleosides. The 5′-wing may consist of, e.g., 1 to 6 linked nucleosides. The 5′-wing may consist of, e.g., 1 to 5 linked nucleosides. The 5′-wing may consist of, e.g., 2 to 5 linked nucleosides. The 5′-wing may consist of, e.g., 3 to 5 linked nucleosides. The 5′-wing may consist of, e.g., 4 or 5 linked nucleosides. The 5′-wing may consist of, e.g., 1 to 4 linked nucleosides. The 5′-wing may consist of, e.g., 1 to 3 linked nucleosides. The 5′-wing may consist of, e.g., 1 or 2 linked nucleosides. The 5′-wing may consist of, e.g., 2 to 4 linked nucleosides. The 5′-wing may consist of, e.g., 2 or 3 linked nucleosides. The 5′-wing may consist of, e.g., 3 or 4 linked nucleosides. The 5′-wing may consist of, e.g., 1 nucleoside. The 5′-wing may consist of, e.g., 2 linked nucleosides. The 5′-wing may consist of, e.g., 3 linked nucleosides. The 5′-wing may consist of, e.g., 4 linked nucleosides. The 5′-wing may consist of, e.g., 5 linked nucleosides. The 5′-wing may consist of, e.g., 6 linked nucleosides.

The 3′-wing may consists of, e.g., 1 to 8 nucleosides. The 3′-wing may consist of, e.g., 1 to 7 nucleosides. The 3′-wing may consist of, e.g., 1 to 6 linked nucleosides. The 3′-wing may consist of, e.g., 1 to 5 linked nucleosides. The 3′-wing may consist of, e.g., 2 to 5 linked nucleosides. The 3′-wing may consist of, e.g., 3 to 5 linked nucleosides. The 3′-wing may consist of, e.g., 4 or 5 linked nucleosides. The 3′-wing may consist of, e.g., 1 to 4 linked nucleosides. The 3′-wing may consist of, e.g., 1 to 3 linked nucleosides. The 3′-wing may consist of, e.g., 1 or 2 linked nucleosides. The 3′-wing may consist of, e.g., 2 to 4 linked nucleosides. The 3′-wing may consist of, e.g., 2 or 3 linked nucleosides. The 3′-wing may consist of, e.g., 3 or 4 linked nucleosides. The 3′-wing may consist of, e.g., 1 nucleoside. The 3′-wing may consist of, e.g., 2 linked nucleosides. The 3′-wing may consist of, e.g., 3 linked nucleosides. The 3′-wing may consist of, e.g., 4 linked nucleosides. The 3′-wing may consist of, e.g., 5 linked nucleosides. The 3′-wing may consist of, e.g., 6 linked nucleosides.

The 5′-wing may include, e.g., at least one bridged nucleoside. The 5′-wing may include, e.g., at least two bridged nucleosides. The 5′-wing may include, e.g., at least three bridged nucleosides. The 5′-wing may include, e.g., at least four bridged nucleosides. The 5′-wing may include, e.g., at least one constrained ethyl (cEt) nucleoside. The 5′-wing may include, e.g., at least one LNA nucleoside. Each nucleoside of the 5′-wing may be, e.g., a bridged nucleoside. Each nucleoside of the 5′-wing may be, e.g., a constrained ethyl (cEt) nucleoside. Each nucleoside of the 5′-wing may be, e.g., a LNA nucleoside.

The 3′-wing may include, e.g., at least one bridged nucleoside. The 3′-wing may include, e.g., at least two bridged nucleosides. The 3′-wing may include, e.g., at least three bridged nucleosides. The 3′-wing may include, e.g., at least four bridged nucleosides. The 3′-wing may include, e.g., at least one constrained ethyl (cEt) nucleoside. The 3′-wing may include, e.g., at least one LNA nucleoside. Each nucleoside of the 3′-wing may be, e.g., a bridged nucleoside. Each nucleoside of the 3′-wing may be, e.g., a constrained ethyl (cEt) nucleoside. Each nucleoside of the 3′-wing may be, e.g., a LNA nucleoside.

The 5′-wing may include, e.g., at least one non-bicyclic modified nucleoside. The 5′-wing may include, e.g., at least one 2′-substituted nucleoside. The 5′-wing may include, e.g., at least one 2′-MOE nucleoside. The 5′-wing may include, e.g., at least one 2′-OMe nucleoside. Each nucleoside of the 5′-wing may be, e.g., a non-bicyclic modified nucleoside. Each nucleoside of the 5′-wing may be, e.g., a 2′-substituted nucleoside. Each nucleoside of the 5′-wing may be, e.g., a 2′-MOE nucleoside. Each nucleoside of the 5′-wing may be, e.g., a 2′-OMe nucleoside.

The 3′-wing may include, e.g., at least one non-bicyclic modified nucleoside. The 3′-wing may include, e.g., at least one 2′-substituted nucleoside. The 3′-wing may include, e.g., at least one 2′-MOE nucleoside. The 3′-wing may include, e.g., at least one 2′-OMe nucleoside. Each nucleoside of the 3′-wing may be, e.g., a non-bicyclic modified nucleoside. Each nucleoside of the 3′-wing may be, e.g., a 2′-substituted nucleoside. Each nucleoside of the 3′-wing may be, e.g., a 2′-MOE nucleoside. Each nucleoside of the 3′-wing may be, e.g., a 2′-OMe nucleoside.

The gap may consist of, e.g., 6 to 20 linked nucleosides. The gap may consist of, e.g., 6 to 15 linked nucleosides. The gap may consist of, e.g., 6 to 12 linked nucleosides. The gap may consist of, e.g., 6 to 10 linked nucleosides. The gap may consist of, e.g., 6 to 9 linked nucleosides. The gap may consist of, e.g., 6 to 8 linked nucleosides. The gap may consist of, e.g., 6 or 7 linked nucleosides. The gap may consist of, e.g., 7 to 10 linked nucleosides. The gap may consist of, e.g., 7 to 9 linked nucleosides. The gap may consist of, e.g., 7 or 8 linked nucleosides. The gap may consist of, e.g., 8 to 10 linked nucleosides. The gap may consist of, e.g., 8 or 9 linked nucleosides. The gap may consist of, e.g., 6 linked nucleosides. The gap may consist of, e.g., 7 linked nucleosides. The gap may consist of, e.g., 8 linked nucleosides. The gap may consist of, e.g., 9 linked nucleosides. The gap may consist of, e.g., 10 linked nucleosides. The gap may consist of, e.g., 11 linked nucleosides. The gap may consist of, e.g., 12 linked nucleosides.

Each nucleoside of the gap may be, e.g., a 2′-deoxynucleoside. The gap may include, e.g., one or more modified nucleosides. Each nucleoside of the gap may be, e.g., a 2′-deoxynucleoside or may be, e.g., a modified nucleoside that is “DNA-like.” In such embodiments, “DNA-like” means that the nucleoside has similar characteristics to DNA, such that a duplex including the gapmer and an RNA molecule is capable of activating RNase H. For example, under certain conditions, 2′-(ara)-F may support RNase H activation, and thus is DNA-like. In certain embodiments, one or more nucleosides of the gap is not a 2′-deoxynucleoside and is not DNA-like. In certain such embodiments, the gapmer nonetheless supports RNase H activation (e.g., by virtue of the number or placement of the non-DNA nucleosides).

In certain embodiments, gaps include a stretch of unmodified 2′-deoxynucleoside interrupted by one or more modified nucleosides, thus resulting in three sub-regions (two stretches of one or more 2′-deoxynucleosides and a stretch of one or more interrupting modified nucleosides). In certain embodiments, no stretch of unmodified 2′-deoxynucleosides is longer than 5, 6, or 7 nucleosides. In certain embodiments, such short stretches is achieved by using short gap regions. In certain embodiments, short stretches are achieved by interrupting a longer gap region.

The gap may include, e.g., one or more modified nucleosides. The gap may include, e.g., one or more modified nucleosides selected from among cEt, FHNA, LNA, and 2-thio-thymidine. The gap may include, e.g., one modified nucleoside. The gap may include, e.g., a 5′-substituted sugar moiety selected from the group consisting of 5′-Me and 5′-(R)-Me. The gap may include, e.g., two modified nucleosides.

The gap may include, e.g., three modified nucleosides. The gap may include, e.g., four modified nucleosides. The gap may include, e.g., two or more modified nucleosides and each modified nucleoside is the same. The gap may include, e.g., two or more modified nucleosides and each modified nucleoside is different.

The gap may include, e.g., one or more modified internucleoside linkages. The gap may include, e.g., one or more methyl phosphonate linkages. In certain embodiments the gap may include, e.g., two or more modified internucleoside linkages. The gap may include, e.g., one or more modified linkages and one or more modified nucleosides. The gap may include, e.g., one modified linkage and one modified nucleoside. The gap may include, e.g., two modified linkages and two or more modified nucleosides.

An antisense oligonucleotide of the invention (e.g., a single-stranded oligonucleotide of the invention) may include one or more mismatches. For example, the mismatch may be specifically positioned within a gapmer, headmer, or tailmer. The mismatch may be, e.g., at position 1, 2, 3, 4, 5, 6, 7, or 8 (e.g., at position 1, 2, 3, or 4) from the 3′-end of the gap region. Alternatively or additionally, the mismatch may be, e.g., at position 9, 8, 7, 6, 5, 4, 3, 2, or 1 (e.g., at position 4, 3, 2, or 1) from the 3′-end of the gap region. In some embodiments, the 5′ wing and/or 3′ wing do not include mismatches.

An antisense oligonucleotide of the invention (e.g., a single-stranded oligonucleotide of the invention) may be a morpholino.

An antisense oligonucleotide of the invention (e.g., a single-stranded oligonucleotide of the invention) may be include a total of X to Y interlinked nucleosides, where X represents the fewest number of nucleosides in the range and Y represents the largest number nucleosides in the range. In these embodiments, X and Y are each independently selected from the group consisting of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X<Y. For example, an oligonucleotide of the invention may include a total of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 interlinked nucleosides.

In some embodiments, an antisense oligonucleotide of the invention (e.g., a single-stranded oligonucleotide of the invention) includes at least one modified internucleoside linkage. A modified internucleoside linkage may be, e.g., a phosphorothioate internucleoside linkage (e.g., a phosphorothioate diester or phosphorothioate triester).

In some embodiments, an antisense oligonucleotide of the invention (e.g., a single-stranded oligonucleotide of the invention) includes at least one stereochemically enriched phosphorothioate-based internucleoside linkage. In some embodiments, an antisense oligonucleotide of the invention (e.g., a single-stranded oligonucleotide of the invention) includes a pattern of stereochemically enriched phosphorothioate internucleoside linkages described herein (e.g., a 5′-R_{P}S_{P}S_{P}-3′). These patterns may enhance target NRL nucleic acid cleavage by RNase H relative to a stereorandom corresponding oligonucleotide. In some embodiments, inclusion and/or location of particular stereochemically enriched linkages within an oligonucleotide may alter the cleavage pattern of a target nucleic acid, when such an oligonucleotide is utilized for cleaving the nucleic acid. For example, a pattern of internucleoside linkage P-stereogenic centers may increase cleavage efficiency of a target nucleic acid. A pattern of internucleoside linkage P-stereogenic centers may provide new cleavage sites in a target nucleic acid. A pattern of internucleoside linkage P-stereogenic centers may reduce the number of cleavage sites, for example, by blocking certain existing cleavage sites. Moreover, in some embodiments, a pattern of internucleoside linkage P-stereogenic centers may facilitate cleavage at only one site within the target sequence that is complementary to an oligonucleotide utilized for the cleavage. Cleavage efficiency may be increased by selecting a pattern of internucleoside linkage P-stereogenic centers that reduces the number of cleavage sites in a target nucleic acid.

Purity of an oligonucleotide may be expressed as the percentage of oligonucleotide molecules that are of the same oligonucleotide type within an oligonucleotide composition. At least about 10% of the oligonucleotides may be, e.g., of the same oligonucleotide type. At least about 20% of the oligonucleotides may be, e.g., of the same oligonucleotide type. At least about 30% of the oligonucleotides may be, e.g., of the same oligonucleotide type. At least about 40% of the oligonucleotides may be, e.g., of the same oligonucleotide type. At least about 50% of the oligonucleotides may be, e.g., of the same oligonucleotide type. At least about 60% of the oligonucleotides may be, e.g., of the same oligonucleotide type. At least about 70% of the oligonucleotides may be, e.g., of the same oligonucleotide type. At least about 80% of the oligonucleotides may be, e.g., of the same oligonucleotide type. At least about 90% of the oligonucleotides may be, e.g., of the same oligonucleotide type. At least about 92% of the oligonucleotides may be, e.g., of the same oligonucleotide type. At least about 94% of the oligonucleotides may be, e.g., of the same oligonucleotide type. At least about 95% of the oligonucleotides may be, e.g., of the same oligonucleotide type. At least about 96% of the oligonucleotides may be, e.g., of the same oligonucleotide type. At least about 97% of the oligonucleotides may be, e.g., of the same oligonucleotide type. At least about 98% of the oligonucleotides may be, e.g., of the same oligonucleotide type. At least about 99% of the oligonucleotides may be, e.g., of the same oligonucleotide type.

An oligonucleotide may include one or more stereochemically enriched internucleoside linkages. An oligonucleotide may include two or more stereochemically enriched internucleoside linkages. An oligonucleotide may include three or more stereochemically enriched internucleoside linkages. An oligonucleotide may include four or more stereochemically enriched internucleoside linkages. An oligonucleotide may include five or more stereochemically enriched internucleoside linkages. An oligonucleotide may include 1, 2, 3, 4, 5, 6, 7, 8, 9, 10, 11, 12, 13, 14, 15, 16, 17, 18, 19, 20, 21, 22, 23, 24, or 25 stereochemically enriched internucleoside linkages. An oligonucleotide may include 5 or more stereochemically enriched internucleoside linkages. An oligonucleotide may include 6 or more stereochemically enriched internucleoside linkages. An oligonucleotide may include 7 or more stereochemically enriched internucleoside linkages. An oligonucleotide may include 8 or more stereochemically enriched internucleoside linkages. An oligonucleotide may include 9 or more stereochemically enriched internucleoside linkages. An oligonucleotide may include 10 or more stereochemically enriched internucleoside linkages. An oligonucleotide may include 11 or more stereochemically enriched internucleoside linkages. An oligonucleotide may include 12 or more stereochemically enriched internucleoside linkages. An oligonucleotide may include 13 or more stereochemically enriched internucleoside linkages. An oligonucleotide may include 14 or more stereochemically enriched internucleoside linkages. An oligonucleotide may include 15 or more stereochemically enriched internucleoside linkages. An oligonucleotide may include 16 or more stereochemically enriched internucleoside linkages. An oligonucleotide may include 17 or more stereochemically enriched internucleoside linkages. An oligonucleotide may include 18 or more stereochemically enriched internucleoside linkages. An oligonucleotide may include 19 or more stereochemically enriched internucleoside linkages. An oligonucleotide may include 20 or more stereochemically enriched internucleoside linkages. An oligonucleotide may include 21 or more stereochemically enriched internucleoside linkages. An oligonucleotide may include 22 or more stereochemically enriched internucleoside linkages. An oligonucleotide may include 23 or more stereochemically enriched internucleoside linkages. An oligonucleotide may include 24 or more stereochemically enriched internucleoside linkages. An oligonucleotide may include 25 or more stereochemically enriched internucleoside linkages.

An oligonucleotide may include at least 5%, 10%, 15%, 20%, 25%, 30%, 35%, 40%, 45%, 50%, 55%, 60%, 65%, 70%, 75%, 80%, 85%, 90%, 95%, or 100% stereochemically enriched internucleoside linkages. Exemplary such stereochemically enriched internucleoside linkages are described herein. An oligonucleotide may include at least 5%, 10%, 15%, 20%, 25%, 30%, 35%, 40%, 45%, 50%, 55%, 60%, 65%, 70%, 75%, 80%, 85%, 90%, 95%, or 100% stereochemically enriched internucleoside linkages in the S_{P} configuration.

A stereochemically enriched internucleoside linkage may be, e.g., a stereochemically enriched phosphorothioate internucleoside linkage. A provided oligonucleotide comprises at least 5%, 10%, 15%, 20%, 25%, 30%, 35%, 40%, 45%, 50%, 55%, 60%, 65%, 70%, 75%, 80%, 85%, 90%, 95%, or 100% stereochemically enriched phosphorothioate internucleoside linkages. All internucleoside linkages may be, e.g., stereochemically enriched phosphorothioate internucleoside linkages. At least 10, 20, 30, 40, 50, 60, 70, 80, or 90% stereochemically enriched phosphorothioate internucleoside linkages have the S_{P} stereochemical configuration. At least 10% stereochemically enriched phosphorothioate internucleoside linkages have the S_{P} stereochemical configuration. At least 20% stereochemically enriched phosphorothioate internucleoside linkages have the S_{P} stereochemical configuration. At least 30% stereochemically enriched phosphorothioate internucleoside linkages have the S_{P} stereochemical configuration. At least 40% stereochemically enriched phosphorothioate internucleoside linkages have the S_{P} stereochemical configuration. At least 50% stereochemically enriched phosphorothioate internucleoside linkages have the S_{P} stereochemical configuration. At least 60% stereochemically enriched phosphorothioate internucleoside linkages have the S_{P} stereochemical configuration. At least 70% stereochemically enriched phosphorothioate internucleoside linkages have the S_{P} stereochemical configuration. At least 80% stereochemically enriched phosphorothioate internucleoside linkages have the S_{P} stereochemical configuration. At least 90% stereochemically enriched phosphorothioate internucleoside linkages have the S_{P} stereochemical configuration. At least 95% stereochemically enriched phosphorothioate internucleoside linkages have the S_{P} stereochemical configuration. At least 10, 20, 30, 40, 50, 60, 70, 80, or 90% stereochemically enriched phosphorothioate internucleoside linkages have the R_{P} stereochemical configuration. At least 10% stereochemically enriched phosphorothioate internucleoside linkages have the R_{P} stereochemical configuration. At least 20% stereochemically enriched phosphorothioate internucleoside linkages have the R_{P} stereochemical configuration. At least 30% stereochemically enriched phosphorothioate internucleoside linkages have the R_{P} stereochemical configuration. At least 40% stereochemically enriched phosphorothioate internucleoside linkages have the R_{P} stereochemical configuration. At least 50% stereochemically enriched phosphorothioate internucleoside linkages have the R_{P} stereochemical configuration. At least 60% stereochemically enriched phosphorothioate internucleoside linkages have the R_{P} stereochemical configuration. At least 70% stereochemically enriched phosphorothioate internucleoside linkages have the R_{P} stereochemical configuration. At least 80% stereochemically enriched phosphorothioate internucleoside linkages have the R_{P} stereochemical configuration. At least 90% stereochemically enriched phosphorothioate internucleoside linkages have the R_{P} stereochemical configuration. At least 95% stereochemically enriched phosphorothioate internucleoside linkages have the R_{P} stereochemical configuration. In some embodiments, less than 10, 20, 30, 40, 50, 60, 70, 80, or 90% stereochemically enriched phosphorothioate internucleoside linkages have the R_{P} stereochemical configuration. In some embodiments, less than 10% stereochemically enriched phosphorothioate internucleoside linkages have the R_{P} stereochemical configuration. In some embodiments, less than 20% stereochemically enriched phosphorothioate internucleoside linkages have the R_{P} stereochemical configuration. In some embodiments, less than 30% stereochemically enriched phosphorothioate internucleoside linkages have the R_{P} stereochemical configuration. In some embodiments, less than 40% stereochemically enriched phosphorothioate internucleoside linkages have the R_{P} stereochemical configuration. In some embodiments, less than 50% stereochemically enriched phosphorothioate internucleoside linkages have the R_{P} stereochemical configuration. In some embodiments, less than 60% stereochemically enriched phosphorothioate internucleoside linkages have the R_{P} stereochemical configuration. In some embodiments, less than 70% stereochemically enriched phosphorothioate internucleoside linkages have the R_{P} stereochemical configuration. In some embodiments, less than 80% stereochemically enriched phosphorothioate internucleoside linkages have the R_{P} stereochemical configuration. In some embodiments, less than 90% stereochemically enriched phosphorothioate internucleoside linkages have the R_{P} stereochemical configuration. In some embodiments, less than 95% stereochemically enriched phosphorothioate internucleoside linkages have the R_{P} stereochemical configuration. An oligonucleotide may have, e.g., only one R_{P} stereochemically enriched phosphorothioate internucleoside linkages. An oligonucleotide may have, e.g., only one R_{P} stereochemically enriched phosphorothioate internucleoside linkages, where all internucleoside linkages are stereochemically enriched phosphorothioate internucleoside linkages. A stereochemically enriched phosphorothioate internucleoside linkage may be, e.g., a stereochemically enriched phosphorothioate diester linkage. In some embodiments, each stereochemically enriched phosphorothioate internucleoside linkage is independently a stereochemically enriched phosphorothioate diester linkage. In some embodiments, each internucleoside linkage is independently a stereochemically enriched phosphorothioate diester linkage. In some embodiments, each internucleoside linkage is independently a stereochemically enriched phosphorothioate diester linkage, and only one is R_{P}.

The gap region may include, e.g., a stereochemically enriched internucleoside linkage. The gap region may include, e.g., stereochemically enriched phosphorothioate internucleoside linkages. The gap region may have, e.g., a repeating pattern of internucleoside linkage stereochemistry. The gap region may have, e.g., a repeating pattern of internucleoside linkage stereochemistry. The gap region may have, e.g., a repeating pattern of internucleoside linkage stereochemistry, where the repeating pattern is (S_{P})ₘR_{P} or R_{P}(S_{P})ₘ, where m is 2, 3, 4, 5, 6, 7 or 8. The gap region may have, e.g., a repeating pattern of internucleoside linkage stereochemistry, where the repeating pattern is (S_{P})ₘR_{P} or R_{P}(S_{P})ₘ, where m is 2, 3, 4, 5, 6, 7 or 8. The gap region may have, e.g., a repeating pattern of internucleoside linkage stereochemistry, where the repeating pattern is (S_{P})ₘR_{P}, where m is 2, 3, 4, 5, 6, 7 or 8. The gap region may have, e.g., a repeating pattern of internucleoside linkage stereochemistry, where the repeating pattern is R_{P}(S_{P})ₘ, where m is 2, 3, 4, 5, 6, 7 or 8. The gap region may have, e.g., a repeating pattern of internucleoside linkage stereochemistry, where the repeating pattern is (S_{P})ₘR_{P} or R_{P}(S_{P})ₘ, where m is 2, 3, 4, 5, 6, 7 or 8. The gap region may have, e.g., a repeating pattern of internucleoside linkage stereochemistry, where the repeating pattern is a motif including at least 33% of internucleoside linkages with the S_{P} stereochemical identify. The gap region may have, e.g., a repeating pattern of internucleoside linkage stereochemistry, where the repeating pattern is a motif including at least 50% of internucleoside linkages with the S_{P} stereochemical identify. The gap region may have, e.g., a repeating pattern of internucleoside linkage stereochemistry, where the repeating pattern is a motif including at least 66% of internucleoside linkages with the S_{P} stereochemical identify. The gap region may have, e.g., a repeating pattern of internucleoside linkage stereochemistry, where the repeating pattern is a repeating triplet motif selected from R_{P}R_{P}S_{P} and S_{P}S_{P}R_{P}. The gap region may have, e.g., a repeating pattern of internucleoside linkage stereochemistry, where the repeating pattern is a repeating R_{P}R_{P}S_{P}. The gap region may have, e.g., a repeating pattern of internucleoside linkage stereochemistry, where the repeating pattern is a repeating S_{P}S_{P}R_{P}.

An oligonucleotide may include a pattern of internucleoside P-stereogenic centers in the gap region including (S_{P})ₘR_{P} or R_{P}(S_{P})ₘ. An oligonucleotide may include a pattern of internucleoside P-stereogenic centers in the gap region including R_{P}(S_{P})ₘ. An oligonucleotide may include a pattern of P-stereogenic centers in the gap region including (S_{P})ₘR_{P}. In some embodiments, m is 2. An oligonucleotide may include a pattern of internucleoside P-stereogenic centers in the gap region including R_{P}(S_{P})₂. An oligonucleotide may include a pattern of internucleoside P-stereogenic centers in the gap region including (S_{P})₂R_{P}(S_{P})₂. An oligonucleotide may include a pattern of internucleoside P-stereogenic centers in the gap region including (R_{P})₂R_{P}(S_{P})₂. An oligonucleotide may include a pattern of internucleoside P-stereogenic centers in the gap region including R_{P}S_{P}R_{P}(S_{P})₂. An oligonucleotide may include a pattern of internucleoside P-stereogenic centers in the gap region including S_{P}R_{P}R_{P}(S_{P})₂. An oligonucleotide may include a pattern of internucleoside P-stereogenic centers in the gap region including (S_{P})₂R_{P}.

An oligonucleotide may include a pattern of internucleoside P-stereogenic centers including (S_{P})ₘR_{P} or R_{P}(S_{P})ₘ. An oligonucleotide may include a pattern of internucleoside P-stereogenic centers including R_{P}(S_{P})ₘ. An oligonucleotide may include a pattern of internucleoside P-stereogenic centers including (S_{P})ₘR_{P}. In some embodiments, m is 2. An oligonucleotide may include a pattern of internucleoside P-stereogenic centers including R_{P}(S_{P})₂. An oligonucleotide may include a pattern of internucleoside P-stereogenic centers including (S_{P})₂R_{P}(S_{P})₂. An oligonucleotide may include a pattern of internucleoside P-stereogenic centers including (R_{P})₂R_{P}(S_{P})₂. An oligonucleotide may include a pattern of internucleoside P-stereogenic centers including R_{P}S_{P}R_{P}(S_{P})₂. An oligonucleotide may include a pattern of internucleoside P-stereogenic centers including S_{P}R_{P}R_{P}(S_{P})₂. An oligonucleotide may include a pattern of internucleoside P-stereogenic centers including (S_{P})₂R_{P}.

In the embodiments of internucleoside P-stereogenic center patterns, m is 2, 3, 4, 5, 6, 7 or 8, unless specified otherwise. In some embodiments of internucleoside P-stereogenic center patterns, m is 3, 4, 5, 6, 7 or 8. In some embodiments of internucleoside P-stereogenic center patterns, m is 4, 5, 6, 7 or 8. In some embodiments of internucleoside P-stereogenic center patterns, m is 5, 6, 7 or 8. In some embodiments of internucleoside P-stereogenic center patterns, m is 6, 7 or 8. In some embodiments of internucleoside P-stereogenic center patterns, m is 7 or 8. In some embodiments of internucleoside P-stereogenic center patterns, m is 2. In some embodiments of internucleoside P-stereogenic center patterns, m is 3. In some embodiments of internucleoside P-stereogenic center patterns, m is 4. In some embodiments of internucleoside P-stereogenic center patterns, m is 5. In some embodiments of internucleoside P-stereogenic center patterns, m is 6. In some embodiments of internucleoside P-stereogenic center patterns, m is 7. In some embodiments of internucleoside P-stereogenic center patterns, m is 8.

A repeating pattern may be, e.g., (S_{P})ₘ(R_{P})ₙ, where n is independently 1, 2, 3, 4, 5, 6, 7 or 8, and m is independently as described herein. An oligonucleotide may include a pattern of internucleoside P-stereogenic centers including (S_{P})ₘ(R_{P})ₙ. An oligonucleotide may include a pattern of internucleoside P-stereogenic centers including (S_{P})ₘ(R_{P})ₙ. A repeating pattern may be, e.g., (R_{P})ₙ(S_{P})ₘ, where n is independently 1, 2, 3, 4, 5, 6, 7 or 8, and m is independently as described herein. An oligonucleotide may include a pattern of internucleoside P-stereogenic centers including (R_{P})ₙ(S_{P})ₘ. An oligonucleotide may include a pattern of internucleoside P-stereogenic centers in the gap region including (R_{P})ₙ(S_{P})ₘ. In some embodiments, (R_{P})ₙ(S_{P})ₘ is (R_{P})(S_{P})₂. In some embodiments, (S_{P})ₙ(R_{P})ₘ is (S_{P})₂(R_{P}).

A repeating pattern may be, e.g., (S_{P})ₘ(R_{P})ₙ(S_{P})ₜ, where each of n and t is independently 1, 2, 3, 4, 5, 6, 7 or 8, and m is as described herein. An oligonucleotide may include a pattern of internucleoside P-stereogenic centers including (S_{P})ₘ(R_{P})ₙ(S_{P})ₜ. An oligonucleotide may include a pattern of internucleoside P-stereogenic centers including (S_{P})ₘ(R_{P})ₙ(S_{P})ₜ. A repeating pattern may be, e.g., (S_{P})ₜ(R_{P})ₙ(S_{P})ₘ, where each of n and t is independently 1, 2, 3, 4, 5, 6, 7 or 8, and m is as described herein. An oligonucleotide may include a pattern of internucleoside P-stereogenic centers including (S_{P})ₜ(R_{P})ₙ(S_{P})ₘ. An oligonucleotide may include a pattern of internucleoside P-stereogenic centers in the gap region including (S_{P})ₜ(R_{P})ₙ(S_{P})ₘ.

A repeating pattern is (Np)ₜ(R_{P})ₙ(S_{P})ₘ, where each of n and t is independently 1, 2, 3, 4, 5, 6, 7 or 8, Np is independently R_{P} or S_{P}, and m is as described herein. An oligonucleotide may include a pattern of internucleoside P-stereogenic centers including (Np)ₜ(R_{P})ₙ(S_{P})ₘ. An oligonucleotide may include a pattern of internucleoside P-stereogenic centers in the gap region including (Np)ₜ(R_{P})ₙ(S_{P})ₘ. A repeating pattern may be, e.g., (Np)ₜ(R_{P})ₙ(S_{P})ₘ, where each of n and t is independently 1, 2, 3, 4, 5, 6, 7 or 8, Np is independently R_{P} or S_{P}, and m is as described herein. An oligonucleotide may include a pattern of internucleoside P-stereogenic centers including (Np)ₜ(R_{P})ₙ(S_{P})ₘ. An oligonucleotide may include a pattern of internucleoside P-stereogenic centers in the gap region including (Np)ₜ(R_{P})ₙ(S_{P})ₘ. In some embodiments, Np is R_{P}. In some embodiments, Np is S_{P}. All Np may be, e.g., same. All Np may be, e.g., S_{P}. At least one Np may be, e.g., different from another Np. In some embodiments, t is 2.

In the embodiments of internucleoside P-stereogenic center patterns, n is 1, 2, 3, 4, 5, 6, 7 or 8. In some embodiments of internucleoside P-stereogenic center patterns, n is 2, 3, 4, 5, 6, 7 or 8. In some embodiments of internucleoside P-stereogenic center patterns, n is 3, 4, 5, 6, 7 or 8. In some embodiments of internucleoside P-stereogenic center patterns, n is 4, 5, 6, 7 or 8. In some embodiments of internucleoside P-stereogenic center patterns, n is 5, 6, 7 or 8. In some embodiments of internucleoside P-stereogenic center patterns, n is 6, 7 or 8. In some embodiments of internucleoside P-stereogenic center patterns, n is 7 or 8. In some embodiments of internucleoside P-stereogenic center patterns, n is 1. In some embodiments of internucleoside P-stereogenic center patterns, n is 2. In some embodiments of internucleoside P-stereogenic center patterns, n is 3. In some embodiments of internucleoside P-stereogenic center patterns, n is 4. In some embodiments of internucleoside P-stereogenic center patterns, n is 5. In some embodiments of internucleoside P-stereogenic center patterns, n is 6. In some embodiments of internucleoside P-stereogenic center patterns, n is 7. In some embodiments of internucleoside P-stereogenic center patterns, n is 8.

In the embodiments of internucleoside P-stereogenic center patterns, t is 1, 2, 3, 4, 5, 6, 7 or 8. In some embodiments of internucleoside P-stereogenic center patterns, t is 2, 3, 4, 5, 6, 7 or 8. In some embodiments of internucleoside P-stereogenic center patterns, t is 3, 4, 5, 6, 7 or 8. In some embodiments of internucleoside P-stereogenic center patterns, t is 4, 5, 6, 7 or 8. In some embodiments of internucleoside P-stereogenic center patterns, t is 5, 6, 7 or 8. In some embodiments of internucleoside P-stereogenic center patterns, t is 6, 7 or 8. In some embodiments of internucleoside P-stereogenic center patterns, t is 7 or 8. In some embodiments of internucleoside P-stereogenic center patterns, t is 1. In some embodiments of internucleoside P-stereogenic center patterns, t is 2. In some embodiments of internucleoside P-stereogenic center patterns, t is 3. In some embodiments of internucleoside P-stereogenic center patterns, t is 4. In some embodiments of internucleoside P-stereogenic center patterns, t is 5. In some embodiments of internucleoside P-stereogenic center patterns, t is 6. In some embodiments of internucleoside P-stereogenic center patterns, t is 7. In some embodiments of internucleoside P-stereogenic center patterns, t is 8.

At least one of m and t may be, e.g., greater than 2. At least one of m and t may be, e.g., greater than 3. At least one of m and t may be, e.g., greater than 4. At least one of m and t may be, e.g., greater than 5. At least one of m and t may be, e.g., greater than 6. At least one of m and t may be, e.g., greater than 7. In some embodiments, each of m and t is greater than 2. In some embodiments, each of m and t is greater than 3. In some embodiments, each of m and t is greater than 4. In some embodiments, each of m and t is greater than 5. In some embodiments, each of m and t is greater than 6. In some embodiments, each of m and t is greater than 7.

In some embodiments of internucleoside P-stereogenic center patterns, n is 1, and at least one of m and t is greater than 1. In some embodiments of internucleoside P-stereogenic center patterns, n is 1 and each of m and t is independent greater than 1. In some embodiments of internucleoside P-stereogenic center patterns, m>n and t>n. In some embodiments, (S_{P})ₘ(R_{P})ₙ(S_{P})ₜ is (S_{P})₂R_{P}(S_{P})₂. In some embodiments, (S_{P})ₜ(R_{P})ₙ(S_{P})ₘ is (S_{P})₂R_{P}(S_{P})₂. In some embodiments, (S_{P})ₜ(R_{P})ₙ(S_{P})ₘ is S_{P}R_{P}(S_{P})₂. In some embodiments, (Np)ₜ(R_{P})ₙ(S_{P})ₘ is (Np)ₜR_{P}(S_{P})ₘ. In some embodiments, (Np)ₜ(R_{P})ₙ(S_{P})ₘ is (Np)₂R_{P}(S_{P})ₘ. In some embodiments, (Np)ₜ(R_{P})ₙ(S_{P})ₘ Is (R_{P})₂R_{P}(S_{P})ₘ. In some embodiments, (Np)ₜ(R_{P})ₙ(S_{P})ₘ is (S_{P})₂R_{P}(S_{P})ₘ. In some embodiments, (Np)ₜ(R_{P})ₙ(S_{P})ₘ is R_{P}S_{P}R_{P}(S_{P})ₘ. In some embodiments, (Np)ₜ(R_{P})ₙ(S_{P})ₘ Is S_{P}R_{P}R_{P}(S_{P})ₘ.

In some embodiments, (S_{P})ₜ(R_{P})ₙ(S_{P})ₘ is S_{P}R_{P}S_{P}S_{P}. In some embodiments, (S_{P})ₜ(R_{P})ₙ(S_{P})ₘ is (S_{P})₂R_{P}(S_{P})₂. In some embodiments, (S_{P})ₜ(R_{P})ₙ(S_{P})ₘ is (S_{P})₃R_{P}(S_{P})₃. In some embodiments, (S_{P})ₜ(R_{P})ₙ(S_{P})ₘ is (S_{P})₄R_{P}(S_{P})₄. In some embodiments, (S_{P})ₜ(R_{P})ₙ(S_{P})ₘ is (S_{P})ₜR_{P}(S_{P})₅. In some embodiments, (S_{P})ₜ(R_{P})ₙ(S_{P})ₘ is S_{P}R_{P}(S_{P})₅. In some embodiments, (S_{P})ₜ(R_{P})ₙ(S_{P})ₘ is (S_{P})₂R_{P}(S_{P})₅. In some embodiments, (S_{P})ₜ(R_{P})ₙ(S_{P})ₘ is (S_{P})₃R_{P}(S_{P})₅. In some embodiments, (S_{P})ₜ(R_{P})ₙ(S_{P})ₘ is (S_{P})₄R_{P}(S_{P})₅. In some embodiments, (S_{P})ₜ(R_{P})ₙ(S_{P})ₘ is (S_{P})₅R_{P}(S_{P})₅.

In some embodiments, (S_{P})ₘ(R_{P})ₙ(S_{P})ₜ is (S_{P})₂R_{P}(S_{P})₂. In some embodiments, (S_{P})ₘ(R_{P})ₙ(S_{P})ₜ is (S_{P})₃R_{P}(S_{P})₃. In some embodiments, (S_{P})ₘ(R_{P})ₙ(S_{P})ₜ is (S_{P})₄R_{P}(S_{P})₄. In some embodiments, (S_{P})ₘ(R_{P})ₙ(S_{P})ₜ is (S_{P})ₘR_{P}(S_{P})₅. In some embodiments, (S_{P})ₘ(R_{P})ₙ(S_{P})ₜ is (S_{P})₂R_{P}(S_{P})₅. In some embodiments, (S_{P})ₘ(R_{P})ₙ(S_{P})ₜ is (S_{P})₃R_{P}(S_{P})₅. In some embodiments, (S_{P})ₘ(R_{P})ₙ(S_{P})ₜ is (S_{P})₄R_{P}(S_{P})₅. In some embodiments, (S_{P})ₘ(R_{P})ₙ(S_{P})ₜ is (S_{P})₅R_{P}(S_{P})₅.

The gap region may include, e.g., at least one R_{P} internucleoside linkage. The gap region may include, e.g., at least one R_{P} phosphorothioate internucleoside linkage. The gap region may include, e.g., at least two R_{P} internucleoside linkages. The gap region may include, e.g., at least two R_{P} phosphorothioate internucleoside linkages. The gap region may include, e.g., at least three R_{P} internucleoside linkages. The gap region may include, e.g., at least three R_{P} phosphorothioate internucleoside linkages. The gap region may include, e.g., at least 4, 5, 6, 7, 8, 9, or 10 R_{P} internucleoside linkages. The gap region may include, e.g., at least 4, 5, 6, 7, 8, 9, or 10 R_{P} phosphorothioate internucleoside linkages.

A gapmer may include a wing-gap-wing motif that is a 5-10-5 motif, where the nucleosides in each wing region are 2′-MOE-modified nucleosides. A wing-gap-wing motif of a gapmer may be, e.g., a 5-10-5 motif where the nucleosides in the gap region are 2′-deoxyribonucleosides. A wing-gap-wing motif of a gapmer may be, e.g., a 5-10-5 motif, where all internucleoside linkages are phosphorothioate internucleoside linkages. A wing-gap-wing motif of a gapmer may be, e.g., a 5-10-5 motif, where all internucleoside linkages are stereochemically enriched phosphorothioate internucleoside linkages. A wing-gap-wing motif of a gapmer may be, e.g., a 5-10-5 motif, where the nucleosides in each wing region are 2′-MOE-modified nucleosides, the nucleosides in the gap region are 2′-deoxyribonucleosides, and all internucleoside linkages are stereochemically enriched phosphorothioate internucleoside linkages.

In certain embodiments, a wing-gap-wing motif is a 5-10-5 motif where the residues at each wing region are not 2′-MOE-modified residues. In certain embodiments, a wing-gap-wing motif is a 5-10-5 motif where the residues in the gap region are 2′-deoxyribonucleotide residues. In certain embodiments, a wing-gap-wing motif is a 5-10-5 motif, where all internucleosidic linkages are phosphorothioate internucleosidic linkages. In certain embodiments, a wing-gap-wing motif is a 5-10-5 motif, where all internucleoside linkages are stereochemically enriched phosphorothioate internucleoside linkages. In certain embodiments, a wing-gap-wing motif is a 5-10-5 motif where the residues at each wing region are not 2′-MOE-modified residues, the residues in the gap region are 2′-deoxyribonucleotide, and all internucleoside linkages are stereochemically enriched phosphorothioate internucleoside linkages.

An oligonucleotide may include a region with at least one of the first, second, third, fifth, seventh, eighth, ninth, eighteenth, nineteenth, and twentieth internucleoside linkages being a P-stereogenic linkage (e.g., phosphorothioate phosphodiester or phosphorothioate phosphotriester). At least two of the first, second, third, fifth, seventh, eighth, ninth, eighteenth, nineteenth and twentieth internucleoside linkages are stereogenic. At least three of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least four of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least five of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least six of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least seven of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least eight of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least nine of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). One of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Two of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Three of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Four of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Five of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Six of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Seven of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Eight of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Nine of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Ten of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester).

An oligonucleotide may include a region with at least one of the first, second, third, fifth, seventh, eighteenth, nineteenth and twentieth internucleoside linkages being P-stereogenic (e.g., phosphorothioate phosphodiester or phosphorothioate phosphotriester). At least two of the first, second, third, fifth, seventh, eighteenth, nineteenth and twentieth internucleoside linkages may be, e.g., P-stereogenic (e.g., phosphorothioate phosphodiester or phosphorothioate phosphotriester). At least three of the first, second, third, fifth, seventh, eighteenth, nineteenth and twentieth internucleoside linkages may be, e.g., P-stereogenic (e.g., phosphorothioate phosphodiester or phosphorothioate phosphotriester). At least four of the first, second, third, fifth, seventh, eighteenth, nineteenth and twentieth internucleoside linkages may be, e.g., P-stereogenic (e.g., phosphorothioate phosphodiester or phosphorothioate phosphotriester). At least five of the first, second, third, fifth, seventh, eighteenth, nineteenth and twentieth internucleoside linkages may be, e.g., P-stereogenic (e.g., phosphorothioate phosphodiester or phosphorothioate phosphotriester). At least six of the first, second, third, fifth, seventh, eighteenth, nineteenth and twentieth internucleoside linkages may be, e.g., P-stereogenic (e.g., phosphorothioate phosphodiester or phosphorothioate phosphotriester). At least seven of the first, second, third, fifth, seventh, eighteenth, nineteenth and twentieth internucleoside linkages may be, e.g., P-stereogenic (e.g., phosphorothioate phosphodiester or phosphorothioate phosphotriester). One of the first, second, third, fifth, seventh, eighteenth, nineteenth and twentieth internucleoside may be, e.g., P-stereogenic (e.g., phosphorothioate phosphodiester or phosphorothioate phosphotriester). Two of the first, second, third, fifth, seventh, eighteenth, nineteenth and twentieth internucleoside linkages may be, e.g., P-stereogenic (e.g., phosphorothioate phosphodiester or phosphorothioate phosphotriester). Three of the first, second, third, fifth, seventh, eighteenth, nineteenth and twentieth internucleoside linkages may be, e.g., P-stereogenic (e.g., phosphorothioate phosphodiester or phosphorothioate phosphotriester). Four of the first, second, third, fifth, seventh, eighteenth, nineteenth and twentieth internucleoside linkages may be, e.g., P-stereogenic (e.g., phosphorothioate phosphodiester or phosphorothioate phosphotriester). Five of the first, second, third, fifth, seventh, eighteenth, nineteenth and twentieth internucleoside linkages may be, e.g., P-stereogenic (e.g., phosphorothioate phosphodiester or phosphorothioate phosphotriester). Six of the first, second, third, fifth, seventh, eighteenth, nineteenth and twentieth internucleoside linkages may be, e.g., P-stereogenic (e.g., phosphorothioate phosphodiester or phosphorothioate phosphotriester). Seven of the first, second, third, fifth, seventh, eighteenth, nineteenth and twentieth internucleoside linkages may be, e.g., P-stereogenic (e.g., phosphorothioate phosphodiester or phosphorothioate phosphotriester). Eight of the first, second, third, fifth, seventh, eighteenth, nineteenth and twentieth internucleoside linkages may be, e.g., P-stereogenic (e.g., phosphorothioate phosphodiester or phosphorothioate phosphotriester).

An oligonucleotide may include a region with at least one of the first, second, third, fifth, seventh, eighth, ninth, eighteenth, nineteenth, and twentieth internucleoside linkages being P-stereogenic (e.g., phosphorothioate phosphodiester or phosphorothioate phosphotriester), and at least one internucleoside linkage being non-stereogenic. An oligonucleotide may include a region in which at least one of the first, second, third, fifth, seventh, eighteenth, nineteenth, and twentieth internucleoside linkages being P-stereogenic (e.g., phosphorothioate phosphodiester or phosphorothioate phosphotriester), and at least one internucleoside linkage being non-stereogenic. At least two internucleoside linkages may be, e.g., non-stereogenic. At least three internucleoside linkages may be, e.g., non-stereogenic. At least four internucleoside linkages may be, e.g., non-stereogenic. At least five internucleoside linkages may be, e.g., non-stereogenic. At least six internucleoside linkages may be, e.g., non-stereogenic. At least seven internucleoside linkages may be, e.g., non-stereogenic. At least eight internucleoside linkages may be, e.g., non-stereogenic. At least nine internucleoside linkages may be, e.g., non-stereogenic. At least 10 internucleoside linkages may be, e.g., non-stereogenic. At least 11 internucleoside linkages may be, e.g., non-stereogenic. At least 12 internucleoside linkages may be, e.g., non-stereogenic. At least 13 internucleoside linkages may be, e.g., non-stereogenic. At least 14 internucleoside linkages may be, e.g., non-stereogenic. At least 15 internucleoside linkages may be, e.g., non-stereogenic. At least 16 internucleoside linkages may be, e.g., non-stereogenic. At least 17 internucleoside linkages may be, e.g., non-stereogenic. At least 18 internucleoside linkages may be, e.g., non-stereogenic. At least 19 internucleoside linkages may be, e.g., non-stereogenic. At least 20 internucleoside linkages may be, e.g., non-stereogenic. In some embodiments, one internucleoside linkage is non-stereogenic. In some embodiments, two internucleoside linkages are non-stereogenic. In some embodiments, three internucleoside linkages are non-stereogenic. In some embodiments, four internucleoside linkages are non-stereogenic. In some embodiments, five internucleoside linkages are non-stereogenic. In some embodiments, six internucleoside linkages are non-stereogenic. In some embodiments, seven internucleoside linkages are non-stereogenic. In some embodiments, eight internucleoside linkages are non-stereogenic. In some embodiments, nine internucleoside linkages are non-stereogenic. In some embodiments, 10 internucleoside linkages are non-stereogenic. In some embodiments, 11 internucleoside linkages are non-stereogenic. In some embodiments, 12 internucleoside linkages are non-stereogenic. In some embodiments, 13 internucleoside linkages are non-stereogenic. In some embodiments, 14 internucleoside linkages are non-stereogenic. In some embodiments, 15 internucleoside linkages are non-stereogenic. In some embodiments, 16 internucleoside linkages are non-stereogenic. In some embodiments, 17 internucleoside linkages are non-stereogenic. In some embodiments, 18 internucleoside linkages are non-stereogenic. In some embodiments, 19 internucleoside linkages are non-stereogenic. In some embodiments, 20 internucleoside linkages are non-stereogenic. An oligonucleotide may include a region in which all internucleoside linkages, except at least one of the first, second, third, fifth, seventh, eighth, ninth, eighteenth, nineteenth and twentieth internucleoside linkages which is P-stereogenic, are non-stereogenic.

An oligonucleotide may include a region with at least one of the first, second, third, fifth, seventh, eighth, ninth, eighteenth, nineteenth, and twentieth internucleoside linkages being P-stereogenic, and at least one internucleoside linkage being phosphate phosphodiester. An oligonucleotide may include a region with at least one of the first, second, third, fifth, seventh, eighteenth, nineteenth, and twentieth internucleoside linkages being P-stereogenic, and at least one internucleoside linkage being phosphate phosphodiester. At least two internucleoside linkages may be, e.g., phosphate phosphodiesters. At least three internucleoside linkages may be, e.g., phosphate phosphodiesters. At least four internucleoside linkages may be, e.g., phosphate phosphodiesters. At least five internucleoside linkages may be, e.g., phosphate phosphodiesters. At least six internucleoside linkages may be, e.g., phosphate phosphodiesters. At least seven internucleoside linkages may be, e.g., phosphate phosphodiesters. At least eight internucleoside linkages may be, e.g., phosphate phosphodiesters. At least nine internucleoside linkages may be, e.g., phosphate phosphodiesters. At least 10 internucleoside linkages may be, e.g., phosphate phosphodiesters. At least 11 internucleoside linkages may be, e.g., phosphate phosphodiesters. At least 12 internucleoside linkages may be, e.g., phosphate phosphodiesters. At least 13 internucleoside linkages may be, e.g., phosphate phosphodiesters. At least 14 internucleoside linkages may be, e.g., phosphate phosphodiesters. At least 15 internucleoside linkages may be, e.g., phosphate phosphodiesters. At least 16 internucleoside linkages may be, e.g., phosphate phosphodiesters. At least 17 internucleoside linkages may be, e.g., phosphate phosphodiesters. At least 18 internucleoside linkages may be, e.g., phosphate phosphodiesters. At least 19 internucleoside linkages may be, e.g., phosphate phosphodiesters. At least 20 internucleoside linkages may be, e.g., phosphate phosphodiesters. In some embodiments, one internucleoside linkage is phosphate phosphodiesters. In some embodiments, two internucleoside linkages are phosphate phosphodiesters. In some embodiments, three internucleoside linkages are phosphate phosphodiesters. In some embodiments, four internucleoside linkages are phosphate phosphodiesters. In some embodiments, five internucleoside linkages are phosphate phosphodiesters. In some embodiments, six internucleoside linkages are phosphate phosphodiesters. In some embodiments, seven internucleoside linkages are phosphate phosphodiesters. In some embodiments, eight internucleoside linkages are phosphate phosphodiesters. In some embodiments, nine internucleoside linkages are phosphate phosphodiesters. In some embodiments, 10 internucleoside linkages are phosphate phosphodiesters. In some embodiments, 11 internucleoside linkages are phosphate phosphodiesters. In some embodiments, 12 internucleoside linkages are phosphate phosphodiesters. In some embodiments, 13 internucleoside linkages are phosphate phosphodiesters. In some embodiments, 14 internucleoside linkages are phosphate phosphodiesters. In some embodiments, 15 internucleoside linkages are phosphate phosphodiesters. In some embodiments, 16 internucleoside linkages are phosphate phosphodiesters. In some embodiments, 17 internucleoside linkages are phosphate phosphodiesters. In some embodiments, 18 internucleoside linkages are phosphate phosphodiesters. In some embodiments, 19 internucleoside linkages are phosphate phosphodiesters. In some embodiments, 20 internucleoside linkages are phosphate phosphodiesters. An oligonucleotide may include a region with all internucleoside linkages, except at least one of the first, second, third, fifth, seventh, eighth, ninth, eighteenth, nineteenth, and twentieth internucleoside linkages being P-stereogenic, being phosphate phosphodiesters.

An oligonucleotide may include a region with at least one of the first, second, third, fifth, seventh, eighth, ninth, eighteenth, nineteenth, and twentieth internucleoside linkages being P-stereogenic, and at least 10% of all internucleoside linkages in the region being non-stereogenic. An oligonucleotide may include a region with at least one of the first, second, third, fifth, seventh, eighteenth, nineteenth, and twentieth internucleoside linkages being P-stereogenic, and at least 10% of all internucleoside linkages in the region being non-stereogenic. At least 20% of all the internucleoside linkages in the region may be, e.g., non-stereogenic. At least 30% of all the internucleoside linkages in the region may be, e.g., non-stereogenic. At least 40% of all the internucleoside linkages in the region may be, e.g., non-stereogenic. At least 50% of all the internucleoside linkages in the region may be, e.g., non-stereogenic. At least 60% of all the internucleoside linkages in the region may be, e.g., non-stereogenic. At least 70% of all the internucleoside linkages in the region may be, e.g., non-stereogenic. At least 80% of all the internucleoside linkages in the region may be, e.g., non-stereogenic. At least 90% of all the internucleoside linkages in the region may be, e.g., non-stereogenic. At least 50% of all the internucleoside linkages in the region may be, e.g., non-stereogenic. A non-stereogenic internucleoside linkage may be, e.g., a phosphate phosphodiester. In some embodiments, each non-stereogenic internucleoside linkage is a phosphate phosphodiester.

The first internucleoside linkage of the region may be, e.g., an S_{P} internucleoside linkage. The first internucleoside linkage of the region may be, e.g., an R_{P} internucleoside linkage. The second internucleoside linkage of the region may be, e.g., an S_{P} internucleoside linkage. The second internucleoside linkage of the region may be, e.g., an R_{P} internucleoside linkage. The third internucleoside linkage of the region may be, e.g., an S_{P} internucleoside linkage. The third internucleoside linkage of the region may be, e.g., an R_{P} internucleoside linkage. The fifth internucleoside linkage of the region may be, e.g., an S_{P} internucleoside linkage. The fifth internucleoside linkage of the region may be, e.g., an R_{P} internucleoside linkage. The seventh internucleoside linkage of the region may be, e.g., an S_{P} internucleoside linkage. The seventh internucleoside linkage of the region may be, e.g., an R_{P} internucleoside linkage. The eighth internucleoside linkage of the region may be, e.g., an S_{P} internucleoside linkage. The eighth internucleoside linkage of the region may be, e.g., an R_{P} internucleoside linkage. The ninth internucleoside linkage of the region may be, e.g., an S_{P} internucleoside linkage. The ninth internucleoside linkage of the region may be, e.g., an R_{P} internucleoside linkage. The eighteenth internucleoside linkage of the region may be, e.g., an S_{P} internucleoside linkage. The eighteenth internucleoside linkage of the region may be, e.g., an R_{P} internucleoside linkage. The nineteenth internucleoside linkage of the region may be, e.g., an S_{P} internucleoside linkage. The nineteenth internucleoside linkage of the region may be, e.g., an R_{P} internucleoside linkage. The twentieth internucleoside linkage of the region may be, e.g., an S_{P} internucleoside linkage. The twentieth internucleoside linkage of the region may be, e.g., an R_{P} internucleoside linkage.

The region may have a length of, e.g., at least 21 bases. The region may have a length of, e.g., 21 bases.

In some embodiments, each stereochemically enriched internucleoside linkage in an oligonucleotide is a phosphorothioate phosphodiester.

An oligonucleotide may have, e.g., at least 25% of its internucleoside linkages in S_{P} configuration. An oligonucleotide may have, e.g., at least 30% of its internucleoside linkages in S_{P} configuration. An oligonucleotide may have, e.g., at least 35% of its internucleoside linkages in S_{P} configuration. An oligonucleotide may have, e.g., at least 40% of its internucleoside linkages in S_{P} configuration. An oligonucleotide may have, e.g., at least 45% of its internucleoside linkages in S_{P} configuration. An oligonucleotide may have, e.g., at least 50% of its internucleoside linkages in S_{P} configuration. An oligonucleotide may have, e.g., at least 55% of its internucleoside linkages in S_{P} configuration. An oligonucleotide may have, e.g., at least 60% of its internucleoside linkages in S_{P} configuration. An oligonucleotide may have, e.g., at least 65% of its internucleoside linkages in S_{P} configuration. An oligonucleotide may have, e.g., at least 70% of its internucleoside linkages in S_{P} configuration. An oligonucleotide may have, e.g., at least 75% of its internucleoside linkages in S_{P} configuration. An oligonucleotide may have, e.g., at least 80% of its internucleoside linkages in S_{P} configuration. An oligonucleotide may have, e.g., at least 85% of its internucleoside linkages in S_{P} configuration. An oligonucleotide may have, e.g., at least 90% of its internucleoside linkages in S_{P} configuration.

An oligonucleotide may include at least two internucleoside linkages having different stereochemical configuration and/or different P-modifications relative to one another. The oligonucleotide may have a structure represented by the following formula:

[S^{B}ₙ₁R^{B}ₙ₂S^{B}ₙ₃R^{B}ₙ₄ . . . S^{B}ₙₓR^{B}_{ny}]

where:

each R^{B} independently represents a block of nucleotide units having the R_{P} configuration at the internucleoside linkage phosphorus atom;

each S^{B} independently represents a block of nucleotide units having the S_{P} configuration at the internucleoside linkage phosphorus atom;

each of n1 to ny is zero or an integer, provided that at least one odd n and at least one even n must be non-zero so that the oligonucleotide includes at least two internucleoside linkages with different stereochemistry relative to one another; and

where the sum of n1 to ny is between 2 and 200.

In some embodiments, the sum of n1 to ny is between a lower limit selected from the group consisting of 2, 3, 4, 5, 6, 7, 8, 9, 10, 11, 12, 13, 14, 15, 16, 17, 18, 19, 20, 21, 22, 23, 24, 25, and more, and the upper limit selected from the group consisting of 5, 10, 15, 20, 25, 30, 35, 40, 45, 50, 55, 60, 65, 70, 75, 80, 85, 90, 95, 100, 110, 120, 130, 140, 150, 160, 170, 180, 190, and 200, the upper limit being greater than the lower limit. In some of these embodiments, each n has the same value. In some embodiments, each even n has the same value as each other even n. In some embodiments, each odd n has the same value each other odd n. At least two even ns may have, e.g., different values from one another. At least two odd ns may have, e.g., different values from one another.

At least two adjacent ns may be, e.g., equal to one another, so that an oligonucleotide includes adjacent blocks of S_{P} linkages and R_{P} linkages of equal lengths. In some embodiments, an oligonucleotide includes repeating blocks of S_{P} and R_{P} linkages of equal lengths. In some embodiments, an oligonucleotide includes repeating blocks of S_{P} and R_{P} linkages, where at least two such blocks are of different lengths from one another. In some such embodiments, each S_{P} block is of the same length and is of a different length from each R_{P} block, where all R_{P} blocks may optionally be of the same length as one another.

At least two skip-adjacent ns may be, e.g., equal to one another, so that a provided oligonucleotide includes at least two blocks of internucleoside linkages of a first stereochemistry that are equal in length to one another and are separated by a separating block of internucleoside linkages of the opposite stereochemistry, where the separating block may be of the same length or a different length from the blocks of first stereochemistry.

In some embodiments, ns associated with linkage blocks at the ends of an oligonucleotide are of the same length. In some embodiments, an oligonucleotide has terminal blocks of the same linkage stereochemistry. In some such embodiments, the terminal blocks are separated from one another by a middle block of the opposite linkage stereochemistry.

An oligonucleotide of formula [S^{B}ₙ₁R^{B}ₙ₂S^{B}ₙ₃R^{B}ₙ₄ . . . S^{B}ₙₓR^{B}_{ny}] may be, e.g., a stereoblockmer. An oligonucleotide of formula [S^{B}ₙ₁R^{B}ₙ₂S^{B}ₙ₃R^{B}ₙ₄ . . . S^{B}ₙₓR^{B}_{ny}] may be, e.g., a stereoskipmer. An oligonucleotide of formula [S^{B}ₙ₁R^{B}ₙ₂S^{B}ₙ₃R^{B}ₙ₄ . . . S^{B}ₙₓR^{B}_{ny}] may be, e.g., a stereoaltmer. An oligonucleotide of formula [S^{B}ₙ₁R^{B}ₙ₂S^{B}ₙ₃R^{B}ₙ₄ . . . S^{B}ₙₓR^{B}_{ny}] may be, e.g., a gapmer.

An oligonucleotide of formula [S^{B}ₙ₁R^{B}ₙ₂S^{B}ₙ₃R^{B}ₙ₄ . . . S^{B}ₙₓR^{B}_{ny}] may be, e.g., of any of the above described patterns and may further include, e.g., patterns of P-modifications. For instance, an oligonucleotide of formula [S^{B}ₙ₁R^{B}ₙ₂S^{B}ₙ₃R^{B}ₙ₄ . . . S^{B}ₙₓR^{B}_{ny}] may be, e.g., a stereoskipmer and a P-modification skipmer. An oligonucleotide of formula [S^{B}ₙ₁R^{B}ₙ₂S^{B}ₙ₃R^{B}ₙ₄ . . . S^{B}ₙₓR^{B}_{ny}] may be, e.g., a stereoblockmer and a P-modification altmer. An oligonucleotide of formula [S^{B}ₙ₁R^{B}ₙ₂S^{B}ₙ₃R^{B}ₙ₄ . . . S^{B}ₙₓR^{B}_{ny}] may be, e.g., a stereoaltmer and a P-modification blockmer.

An oligonucleotide may include, e.g., at least one phosphate phosphodiester and at least two consecutive modified internucleoside linkages. An oligonucleotide may include, e.g., at least one phosphate phosphodiester and at least two consecutive phosphorothioate triesters.

An oligonucleotide may be, e.g., a blockmer. An oligonucleotide may be, e.g., a stereoblockmer. An oligonucleotide may be, e.g., a P-modification blockmer. An oligonucleotide may be, e.g., a linkage blockmer.

An oligonucleotide may be, e.g., an altmer. An oligonucleotide may be, e.g., a stereoaltmer. An oligonucleotide may be, e.g., a P-modification altmer. An oligonucleotide may be, e.g., a linkage altmer.

An oligonucleotide may be, e.g., a unimer. An oligonucleotide may be, e.g., a stereounimer. An oligonucleotide may be, e.g., a P-modification unimer. An oligonucleotide may be, e.g., a linkage unimer.

An oligonucleotide may be, e.g., a skipmer.

### II. RNAi

In another approach, the invention provides a double-stranded oligonucleotide including a passenger strand hybridized to a guide strand having a nucleobase sequence with at least 6 contiguous nucleobases complementary to an equal-length portion within an NRL target nucleic acid. This approach is typically referred to as an RNAi approach, and the corresponding oligonucleotides of the invention are referred to as siRNA. Without wishing to be bound by theory, this approach involves incorporation of the guide strand into an RNA-induced silencing complex (RISC), which can identify and hybridize to an NRL target nucleic acid in a cell through complementarity of a portion of the guide strand and the target nucleic acid. Upon identification (and hybridization), RISC may either remain on the target nucleic acid thereby sterically blocking translation or cleave the target nucleic acid.

A double-stranded oligonucleotide of the invention may be an siRNA of the invention. An siRNA of the invention includes a guide strand and a passenger strand that are not covalently linked to each other. Alternatively, a double-stranded oligonucleotide of the invention may be an shRNA of the invention. An shRNA of the invention includes a guide strand and a passenger strand that are covalently linked to each other by a linker. Without wishing to be bound by theory, shRNA is processed by the Dicer enzyme to remove the linker and produce a corresponding siRNA.

A double-stranded oligonucleotide of the invention (e.g., an siRNA of the invention) includes a nucleobase sequence having at least 6 (e.g., at least 7, 8, 9, 10, 11, 12, 13, 14, 15, 16, 17, 18, 19, or 20) contiguous nucleobases complementary to an equal-length portion within an NRL target nucleic acid. The equal-length portion within an NRL target nucleic acid may be, e.g., a coding sequence within the NRL target nucleic acid. The NRL target nucleic acid may be NRL pre-mRNA, NRL transcript 1, NRL transcript 2, NRL transcript 3, or NRL transcript 4. The equal-length portion may include positions 586-605 or 264-283 in NRL transcript 1. The equal-length portion may include positions 815-834 or 965-984 in NRL transcript 1. Non-limiting examples of the equal-length portions include aatgactttgacttgatgaag (positions 586 et seq. of NRL transcript 1) and aactggacagcggagcacgat (positions 264 et seq. of NRL transcript 1). Further non-limiting examples of the equal-length portions include tgagtcctgaagaggccat (positions 815 et seq. of NRL transcript 1) and tgtctgtgcgggagctaaa (positions 965 et seq. of NRL transcript 1).

Typically, a guide strand includes a seed region, a slicing site, and 5′- and 3′-terminal residues. The seed region-typically, a six nucleotide-long sequence from position 2 to position 7—are involved in the target nucleic acid recognition. The slicing site are the nucleotides (typically at positions 10 and 11) that are complementary to the target nucleosides linked by an internucleoside linkage that undergoes a RISC-mediated cleavage. The 5′- and 3′ terminal residues typically interact with or are blocked by the Ago2 component of RISC.

A double-stranded oligonucleotide of the invention (e.g., an siRNA of the invention) may include one or more mismatches. For example, the one or more mismatches may be included outside the seed region and the slicing site. Typically, the one or more mismatches may be included among the 5′- and/or 3′-terminal nucleosides.

A double-stranded oligonucleotide of the invention (e.g., an siRNA of the invention) may include a guide strand having total of X to Y interlinked nucleosides and a passenger strand having a total of X to Y interlinked nucleosides, where each X represents independently the fewest number of nucleosides in the range and each Y represents independently the largest number nucleosides in the range. In these embodiments, X and Y are each independently selected from the group consisting of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X<Y. For example, a strand (e.g., a guide strand or a passenger strand) in a double-stranded oligonucleotide of the invention may include a total of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 interlinked nucleosides.

### III. Complementarity

It is possible to introduce mismatch bases without eliminating activity. Accordingly an oligonucleotide of the invention may include (i) a nucleobase sequence having at least 6 contiguous nucleobases complementary to an equal-length portion within an NRL target nucleic acid and (ii) a nucleobase sequence having a plurality of nucleobases including one or more nucleobases complementary to an NRL target nucleic acid and one or more mismatches.

In some embodiments, oligonucleotides of the invention are complementary to an NRL target nucleic acid over the entire length of the oligonucleotide. In other embodiments, oligonucleotides are 99%, 95%, 90%, 85%, or 80% complementary to the NRL target nucleic acid. In further embodiments, oligonucleotides are at least 80% complementary to the NRL target nucleic acid over the entire length of the oligonucleotide and include a nucleobase sequence that is fully complementary to an NRL target nucleic acid. The nucleobase sequence that is fully complementary may be, e.g., 6 to 20, 10 to 18, or 18 to 20 contiguous nucleobases in length.

An oligonucleotide of the invention may include one or more mismatched nucleobases relative to the target nucleic acid. In certain embodiments, an antisense or RNAi activity against the target is reduced by such mismatch, but activity against a non-target is reduced by a greater amount. Thus, the off-target selectivity of the oligonucleotides may be improved.

### IV. Oligonucleotide Modifications

An oligonucleotide of the invention may be a modified oligonucleotide. A modified oligonucleotide of the invention includes one or more modifications, e.g., a nucleobase modification, a sugar modification, an internucleoside linkage modification, or a terminal modification.

Nucleobase Modifications

Oligonucleotides of the invention may include one or more modified nucleobases. Unmodified nucleobases include the purine bases adenine (A) and guanine (G), and the pyrimidine bases thymine (T), cytosine (C), and uracil (U). Modified nucleobases include 5-substituted pyrimidines, 6-azapyrimidines, alkyl or alkynyl substituted pyrimidines, alkyl substituted purines, and N-2, N-6 and 0-6 substituted purines, as well as synthetic and natural nucleobases, e.g., 5-methylcytosine, 5-hydroxymethyl cytosine, xanthine, hypoxanthine, 2-aminoadenine, 6-alkyl (e.g., 6-methyl) adenine and guanine, 2-alkyl (e.g., 2-propyl) adenine and guanine, 2-thiouracil, 2-thiothymine, 2-thiocytosine, 5-halouracil, 5-halocytosine, 5-propynyl uracil, 5-propynyl cytosine, 5-trifluoromethyl uracil, 5-trifluoromethyl cytosine, 7-methyl guanine, 7-methyl adenine, 8-azaguanine, 8-azaadenine, 7-deazaguanine, 7-deazaadenine, 3-deazaguanine, 3-deazaadenine. Certain nucleobases are particularly useful for increasing the binding affinity of nucleic acids, e g., 5-substituted pyrimidines; 6-azapyrimidines; N2-, N6-, and/or 06-substituted purines. Nucleic acid duplex stability can be enhanced using, e.g., 5-methylcytosine. Non-limiting examples of nucleobases include: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH3) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deazaadenine, 7-deazaguanine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443

Sugar Modifications

Oligonucleotides of the invention may include one or more sugar modifications in nucleosides. Nucleosides having an unmodified sugar include a sugar moiety that is a furanose ring as found in ribonucleosides and 2′-deoxyribonucleosides.

Sugars included in the nucleosides of the invention may be non-furanose (or 4′-substituted furanose) rings or ring systems or open systems. Such structures include simple changes relative to the natural furanose ring (e.g., a six-membered ring). Alternative sugars may also include sugar surrogates wherein the furanose ring has been replaced with another ring system such as, e.g., a morpholino or hexitol ring system. Non-limiting examples of sugar moieties useful that may be included in the oligonucleotides of the invention include β-D-ribose, β-D-2′-deoxyribose, substituted sugars (e.g., 2′, 5′, and bis substituted sugars), 4′-S-sugars (e.g., 4′-S-ribose, 4′-S-2′-deoxyribose, and 4′-S-2′-substituted ribose), bicyclic sugar moieties (e.g., the 2′-O—CH₂-4′ or 2′-O—(CH₂)₂-4′ bridged ribose derived bicyclic sugars) and sugar surrogates (when the ribose ring has been replaced with a morpholino or a hexitol ring system).

Typically, a sugar modification may be, e.g., a 2′-substitution, locking, carbocyclization, or unlocking. A 2′-substitution is a replacement of 2′-hydroxyl in ribofuranose with 2′-fluoro, 2′-methoxy, or 2′-(2-methoxy)ethoxy. Alternatively, a 2′-substitution may be a 2′-(ara) substitution, which corresponds to the following structure:

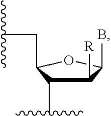

where B is a nucleobase, and R is a 2′-(ara) substituent (e.g., fluoro). 2′-(ara) substituents are known in the art and can be same as other 2′-substituents described herein. In some embodiments, 2′-(ara) substituent is a 2′-(ara)-F substituent (R is fluoro). A locking modification is an incorporation of a bridge between 4′-carbon atom and 2′-carbon atom of ribofuranose. Nucleosides having a sugar with a locking modification are known in the art as bridged nucleic acids, e.g., locked nucleic acids (LNA), ethylene-bridged nucleic acids (ENA), and cEt nucleic acids. The bridged nucleic acids are typically used as affinity enhancing nucleosides.

Internucleoside Linkage Modifications

Oligonucleotides of the invention may include one or more internucleoside linkage modifications. The two main classes of internucleoside linkages are defined by the presence or absence of a phosphorus atom. Non-limiting examples of phosphorus-containing internucleoside linkages include phosphodiester linkages, phosphotriester linkages, phosphorothioate diester linkages, phosphorothioate triester linkages, morpholino internucleoside linkages, methylphosphonates, and phosphoramidate. Non-limiting examples of non-phosphorus internucleoside linkages include methylenemethylimino (—CH2-N(CH3)-O—CH2-), thiodiester (—O—C(O)—S—), thionocarbamate (—O—C(O)(NH)—S—), siloxane (—O—Si(H)₂—O—), and N,N′-dimethylhydrazine (—CH2-N(CH3)-N(CH3)-). Modified linkages, compared to natural phosphodiester linkages, can be used to alter, typically increase, nuclease resistance of the oligonucleotide. Methods of preparation of phosphorous-containing and non-phosphorous-containing internucleoside linkages are known in the art.

Internucleoside linkages may be stereochemically enriched. For example, phosphorothioate-based internucleoside linkages (e.g., phosphorothioate diester or phosphorothioate triester) may be stereochemically enriched. The stereochemically enriched internucleoside linkages including a stereogenic phosphorus are typically designated S_{P} or R_{P} to identify the absolute stereochemistry of the phosphorus atom. Within an oligonucleotide, S_{P} phosphorothioate indicates the following structure:

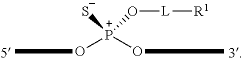

Within an oligonucleotide, R_{P} phosphorothioate indicates the following structure:

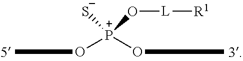

The oligonucleotides of the invention may include one or more neutral internucleoside linkages. Non-limiting examples of neutral internucleoside linkages include phosphotriesters, phosphorothioate triesters, methylphosphonates, methylenemethylimino (3′-CH₂—N(CH₃)—O—3′), amide-3 (3′-CH₂—C(═O)—N(H)-3′), amide-4 (3′-CH2-N(H)—C(═O)-3′), formacetal (3′-O—CH2-O—3′), and thioformacetal (3′-S—CH₂—O—3′). Further neutral internucleoside linkages include nonionic linkages including siloxane (dialkylsiloxane), carboxylate ester, carboxamide, sulfide, sulfonate ester, and amides (See for example: Carbohydrate Modifications in Antisense Research; Y. S. Sanghvi and P. D. Cook, Eds., ACS Symposium Series 580; Chapters 3 and 4, 40-65).

Oligonucleotides may include, e.g., modified internucleoside linkages arranged along the oligonucleotide or region thereof in a defined pattern or modified internucleoside linkage motif. Oligonucleotides may include, e.g., a region having an alternating internucleoside linkage motif. In certain embodiments, oligonucleotides of the present disclosure include a region of uniformly modified internucleoside linkages. In certain such embodiments, the oligonucleotide may include, e.g., a region that is uniformly linked by phosphorothioate internucleoside linkages. The oligonucleotide may be, e.g., uniformly linked by phosphorothioate internucleoside linkages. Each internucleoside linkage of the oligonucleotide is selected from phosphodiester and phosphorothioate. Each internucleoside linkage of the oligonucleotide is selected from phosphodiester and phosphorothioate and at least one internucleoside linkage is phosphorothioate.

The oligonucleotide may include, e.g., at least 6 phosphorothioate internucleoside linkages. The oligonucleotide may include, e.g., at least 7 phosphorothioate internucleoside linkages. The oligonucleotide may include, e.g., at least 8 phosphorothioate internucleoside linkages. The oligonucleotide may include, e.g., at least 9 phosphorothioate internucleoside linkages. The oligonucleotide may include, e.g., at least 10 phosphorothioate internucleoside linkages. The oligonucleotide may include, e.g., at least 11 phosphorothioate internucleoside linkages. The oligonucleotide may include, e.g., at least 12 phosphorothioate internucleoside linkages. The oligonucleotide may include, e.g., at least 13 phosphorothioate internucleoside linkages. The oligonucleotide may include, e.g., at least 14 phosphorothioate internucleoside linkages.

The oligonucleotide may include, e.g., at least one block of at least 6 consecutive phosphorothioate internucleoside linkages. The oligonucleotide may include, e.g., at least one block of at least 7 consecutive phosphorothioate internucleoside linkages. The oligonucleotide may include, e.g., at least one block of at least 8 consecutive phosphorothioate internucleoside linkages. The oligonucleotide may include, e.g., at least one block of at least 9 consecutive phosphorothioate internucleoside linkages. The oligonucleotide may include, e.g., at least one block of at least 10 consecutive phosphorothioate internucleoside linkages. The oligonucleotide may include, e.g., at least one block of at least 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide. The oligonucleotide may include, e.g., fewer than 15 phosphorothioate internucleoside linkages. The oligonucleotide may include, e.g., fewer than 14 phosphorothioate internucleoside linkages. The oligonucleotide may include, e.g., fewer than 13 phosphorothioate internucleoside linkages. The oligonucleotide may include, e.g., fewer than 12 phosphorothioate internucleoside linkages. The oligonucleotide may include, e.g., fewer than 11 phosphorothioate internucleoside linkages. The oligonucleotide may include, e.g., fewer than 10 phosphorothioate internucleoside linkages. The oligonucleotide may include, e.g., fewer than 9 phosphorothioate internucleoside linkages. The oligonucleotide may include, e.g., fewer than 8 phosphorothioate internucleoside linkages. The oligonucleotide may include, e.g., fewer than 7 phosphorothioate internucleoside linkages. The oligonucleotide may include, e.g., fewer than 6 phosphorothioate internucleoside linkages. The oligonucleotide may include, e.g., fewer than 5 phosphorothioate internucleoside linkages. In some embodiments, at least one phosphorothioate internucleoside linkage is a phosphorothioate diester. In some embodiments, each phosphorothioate internucleoside linkage is a phosphorothioate diester. In some embodiments, at least one phosphorothioate internucleoside linkage is a phosphorothioate triester. In some embodiments, each phosphorothioate internucleoside linkage is a phosphorothioate triester. In some embodiments, each internucleoside linkage is independently a phosphodiester (e.g., phosphate phosphodiester or phosphorothioate diester).

An oligonucleotide may include a pattern of internucleoside P-stereogenic centers including (S_{P})ₘR_{P} or R_{P}(S_{P})ₘ. An oligonucleotide may include a pattern of internucleoside P-stereogenic centers including R_{P}(S_{P})ₘ. An oligonucleotide may include a pattern of internucleoside P-stereogenic centers including (S_{P})ₘR_{P}. In some embodiments, m is 2. An oligonucleotide may include a pattern of internucleoside P-stereogenic centers including R_{P}(S_{P})₂. An oligonucleotide may include a pattern of internucleoside P-stereogenic centers including (S_{P})₂R_{P}(S_{P})₂. An oligonucleotide may include a pattern of internucleoside P-stereogenic centers including (R_{P})₂R_{P}(S_{P})₂. An oligonucleotide may include a pattern of internucleoside P-stereogenic centers including R_{P}S_{P}R_{P}(S_{P})₂. An oligonucleotide may include a pattern of internucleoside P-stereogenic centers including S_{P}R_{P}R_{P}(S_{P})₂. An oligonucleotide may include a pattern of internucleoside P-stereogenic centers including (S_{P})₂R_{P}.

In the embodiments of internucleoside P-stereogenic center patterns, m is 2, 3, 4, 5, 6, 7 or 8, unless specified otherwise. In some embodiments of internucleoside P-stereogenic center patterns, m is 3, 4, 5, 6, 7 or 8. In some embodiments of internucleoside P-stereogenic center patterns, m is 4, 5, 6, 7 or 8. In some embodiments of internucleoside P-stereogenic center patterns, m is 5, 6, 7 or 8. In some embodiments of internucleoside P-stereogenic center patterns, m is 6, 7 or 8. In some embodiments of internucleoside P-stereogenic center patterns, m is 7 or 8. In some embodiments of internucleoside P-stereogenic center patterns, m is 2. In some embodiments of internucleoside P-stereogenic center patterns, m is 3. In some embodiments of internucleoside P-stereogenic center patterns, m is 4. In some embodiments of internucleoside P-stereogenic center patterns, m is 5. In some embodiments of internucleoside P-stereogenic center patterns, m is 6. In some embodiments of internucleoside P-stereogenic center patterns, m is 7. In some embodiments of internucleoside P-stereogenic center patterns, m is 8.

An oligonucleotide may include a region with at least one of the first, second, third, fifth, seventh, eighth, ninth, eighteenth, nineteenth, and twentieth internucleoside linkages being a P-stereogenic linkage (e.g., phosphorothioate phosphodiester or phosphorothioate phosphotriester). At least two of the first, second, third, fifth, seventh, eighth, ninth, eighteenth, nineteenth and twentieth internucleoside linkages are stereogenic. At least three of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least four of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least five of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least six of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least seven of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least eight of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least nine of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). One of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Two of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Three of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Four of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Five of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Six of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Seven of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Eight of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Nine of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Ten of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester).

An oligonucleotide may include a region with at least one of the first, second, third, fifth, seventh, eighteenth, nineteenth and twentieth internucleoside linkages being P-stereogenic (e.g., phosphorothioate phosphodiester or phosphorothioate phosphotriester). At least two of the first, second, third, fifth, seventh, eighteenth, nineteenth and twentieth internucleoside linkages may be, e.g., P-stereogenic (e.g., phosphorothioate phosphodiester or phosphorothioate phosphotriester). At least three of the first, second, third, fifth, seventh, eighteenth, nineteenth and twentieth internucleoside linkages may be, e.g., P-stereogenic (e.g., phosphorothioate phosphodiester or phosphorothioate phosphotriester). At least four of the first, second, third, fifth, seventh, eighteenth, nineteenth and twentieth internucleoside linkages may be, e.g., P-stereogenic (e.g., phosphorothioate phosphodiester or phosphorothioate phosphotriester). At least five of the first, second, third, fifth, seventh, eighteenth, nineteenth and twentieth internucleoside linkages may be, e.g., P-stereogenic (e.g., phosphorothioate phosphodiester or phosphorothioate phosphotriester). At least six of the first, second, third, fifth, seventh, eighteenth, nineteenth and twentieth internucleoside linkages may be, e.g., P-stereogenic (e.g., phosphorothioate phosphodiester or phosphorothioate phosphotriester). At least seven of the first, second, third, fifth, seventh, eighteenth, nineteenth and twentieth internucleoside linkages may be, e.g., P-stereogenic (e.g., phosphorothioate phosphodiester or phosphorothioate phosphotriester). One of the first, second, third, fifth, seventh, eighteenth, nineteenth and twentieth internucleoside may be, e.g., P-stereogenic (e.g., phosphorothioate phosphodiester or phosphorothioate phosphotriester). Two of the first, second, third, fifth, seventh, eighteenth, nineteenth and twentieth internucleoside linkages may be, e.g., P-stereogenic (e.g., phosphorothioate phosphodiester or phosphorothioate phosphotriester). Three of the first, second, third, fifth, seventh, eighteenth, nineteenth and twentieth internucleoside linkages may be, e.g., P-stereogenic (e.g., phosphorothioate phosphodiester or phosphorothioate phosphotriester). Four of the first, second, third, fifth, seventh, eighteenth, nineteenth and twentieth internucleoside linkages may be, e.g., P-stereogenic (e.g., phosphorothioate phosphodiester or phosphorothioate phosphotriester). Five of the first, second, third, fifth, seventh, eighteenth, nineteenth and twentieth internucleoside linkages may be, e.g., P-stereogenic (e.g., phosphorothioate phosphodiester or phosphorothioate phosphotriester). Six of the first, second, third, fifth, seventh, eighteenth, nineteenth and twentieth internucleoside linkages may be, e.g., P-stereogenic (e.g., phosphorothioate phosphodiester or phosphorothioate phosphotriester). Seven of the first, second, third, fifth, seventh, eighteenth, nineteenth and twentieth internucleoside linkages may be, e.g., P-stereogenic (e.g., phosphorothioate phosphodiester or phosphorothioate phosphotriester). Eight of the first, second, third, fifth, seventh, eighteenth, nineteenth and twentieth internucleoside linkages may be, e.g., P-stereogenic (e.g., phosphorothioate phosphodiester or phosphorothioate phosphotriester).

An oligonucleotide may include a region with at least one of the first, second, third, fifth, seventh, eighth, ninth, eighteenth, nineteenth, and twentieth internucleoside linkages being P-stereogenic (e.g., phosphorothioate phosphodiester or phosphorothioate phosphotriester), and at least one internucleoside linkage being non-stereogenic. An oligonucleotide may include a region in which at least one of the first, second, third, fifth, seventh, eighteenth, nineteenth, and twentieth internucleoside linkages being P-stereogenic (e.g., phosphorothioate phosphodiester or phosphorothioate phosphotriester), and at least one internucleoside linkage being non-stereogenic. At least two internucleoside linkages may be, e.g., non-stereogenic. At least three internucleoside linkages may be, e.g., non-stereogenic. At least four internucleoside linkages may be, e.g., non-stereogenic. At least five internucleoside linkages may be, e.g., non-stereogenic. At least six internucleoside linkages may be, e.g., non-stereogenic. At least seven internucleoside linkages may be, e.g., non-stereogenic. At least eight internucleoside linkages may be, e.g., non-stereogenic. At least nine internucleoside linkages may be, e.g., non-stereogenic. At least 10 internucleoside linkages may be, e.g., non-stereogenic. At least 11 internucleoside linkages may be, e.g., non-stereogenic. At least 12 internucleoside linkages may be, e.g., non-stereogenic. At least 13 internucleoside linkages may be, e.g., non-stereogenic. At least 14 internucleoside linkages may be, e.g., non-stereogenic. At least 15 internucleoside linkages may be, e.g., non-stereogenic. At least 16 internucleoside linkages may be, e.g., non-stereogenic. At least 17 internucleoside linkages may be, e.g., non-stereogenic. At least 18 internucleoside linkages may be, e.g., non-stereogenic. At least 19 internucleoside linkages may be, e.g., non-stereogenic. At least 20 internucleoside linkages may be, e.g., non-stereogenic. In some embodiments, one internucleoside linkage is non-stereogenic. In some embodiments, two internucleoside linkages are non-stereogenic. In some embodiments, three internucleoside linkages are non-stereogenic. In some embodiments, four internucleoside linkages are non-stereogenic. In some embodiments, five internucleoside linkages are non-stereogenic. In some embodiments, six internucleoside linkages are non-stereogenic. In some embodiments, seven internucleoside linkages are non-stereogenic. In some embodiments, eight internucleoside linkages are non-stereogenic. In some embodiments, nine internucleoside linkages are non-stereogenic. In some embodiments, 10 internucleoside linkages are non-stereogenic. In some embodiments, 11 internucleoside linkages are non-stereogenic. In some embodiments, 12 internucleoside linkages are non-stereogenic. In some embodiments, 13 internucleoside linkages are non-stereogenic. In some embodiments, 14 internucleoside linkages are non-stereogenic. In some embodiments, 15 internucleoside linkages are non-stereogenic. In some embodiments, 16 internucleoside linkages are non-stereogenic. In some embodiments, 17 internucleoside linkages are non-stereogenic. In some embodiments, 18 internucleoside linkages are non-stereogenic. In some embodiments, 19 internucleoside linkages are non-stereogenic. In some embodiments, 20 internucleoside linkages are non-stereogenic. An oligonucleotide may include a region in which all internucleoside linkages, except at least one of the first, second, third, fifth, seventh, eighth, ninth, eighteenth, nineteenth and twentieth internucleoside linkages which is P-stereogenic, are non-stereogenic.

An oligonucleotide may include a region with at least one of the first, second, third, fifth, seventh, eighth, ninth, eighteenth, nineteenth, and twentieth internucleoside linkages being P-stereogenic, and at least one internucleoside linkage being phosphate phosphodiester. An oligonucleotide may include a region with at least one of the first, second, third, fifth, seventh, eighteenth, nineteenth, and twentieth internucleoside linkages being P-stereogenic, and at least one internucleoside linkage being phosphate phosphodiester. At least two internucleoside linkages may be, e.g., phosphate phosphodiesters. At least three internucleoside linkages may be, e.g., phosphate phosphodiesters. At least four internucleoside linkages may be, e.g., phosphate phosphodiesters. At least five internucleoside linkages may be, e.g., phosphate phosphodiesters. At least six internucleoside linkages may be, e.g., phosphate phosphodiesters. At least seven internucleoside linkages may be, e.g., phosphate phosphodiesters. At least eight internucleoside linkages may be, e.g., phosphate phosphodiesters. At least nine internucleoside linkages may be, e.g., phosphate phosphodiesters. At least 10 internucleoside linkages may be, e.g., phosphate phosphodiesters. At least 11 internucleoside linkages may be, e.g., phosphate phosphodiesters. At least 12 internucleoside linkages may be, e.g., phosphate phosphodiesters. At least 13 internucleoside linkages may be, e.g., phosphate phosphodiesters. At least 14 internucleoside linkages may be, e.g., phosphate phosphodiesters. At least 15 internucleoside linkages may be, e.g., phosphate phosphodiesters. At least 16 internucleoside linkages may be, e.g., phosphate phosphodiesters. At least 17 internucleoside linkages may be, e.g., phosphate phosphodiesters. At least 18 internucleoside linkages may be, e.g., phosphate phosphodiesters. At least 19 internucleoside linkages may be, e.g., phosphate phosphodiesters. At least 20 internucleoside linkages may be, e.g., phosphate phosphodiesters. In some embodiments, one internucleoside linkage is phosphate phosphodiesters. In some embodiments, two internucleoside linkages are phosphate phosphodiesters. In some embodiments, three internucleoside linkages are phosphate phosphodiesters. In some embodiments, four internucleoside linkages are phosphate phosphodiesters. In some embodiments, five internucleoside linkages are phosphate phosphodiesters. In some embodiments, six internucleoside linkages are phosphate phosphodiesters. In some embodiments, seven internucleoside linkages are phosphate phosphodiesters. In some embodiments, eight internucleoside linkages are phosphate phosphodiesters. In some embodiments, nine internucleoside linkages are phosphate phosphodiesters. In some embodiments, 10 internucleoside linkages are phosphate phosphodiesters. In some embodiments, 11 internucleoside linkages are phosphate phosphodiesters. In some embodiments, 12 internucleoside linkages are phosphate phosphodiesters. In some embodiments, 13 internucleoside linkages are phosphate phosphodiesters. In some embodiments, 14 internucleoside linkages are phosphate phosphodiesters. In some embodiments, 15 internucleoside linkages are phosphate phosphodiesters. In some embodiments, 16 internucleoside linkages are phosphate phosphodiesters. In some embodiments, 17 internucleoside linkages are phosphate phosphodiesters. In some embodiments, 18 internucleoside linkages are phosphate phosphodiesters. In some embodiments, 19 internucleoside linkages are phosphate phosphodiesters. In some embodiments, 20 internucleoside linkages are phosphate phosphodiesters. An oligonucleotide may include a region with all internucleoside linkages, except at least one of the first, second, third, fifth, seventh, eighth, ninth, eighteenth, nineteenth, and twentieth internucleoside linkages being P-stereogenic, being phosphate phosphodiesters.

An oligonucleotide may include a region with at least one of the first, second, third, fifth, seventh, eighth, ninth, eighteenth, nineteenth, and twentieth internucleoside linkages being P-stereogenic, and at least 10% of all internucleoside linkages in the region being non-stereogenic. An oligonucleotide may include a region with at least one of the first, second, third, fifth, seventh, eighteenth, nineteenth, and twentieth internucleoside linkages being P-stereogenic, and at least 10% of all internucleoside linkages in the region being non-stereogenic. At least 20% of all the internucleoside linkages in the region may be, e.g., non-stereogenic. At least 30% of all the internucleoside linkages in the region may be, e.g., non-stereogenic. At least 40% of all the internucleoside linkages in the region may be, e.g., non-stereogenic. At least 50% of all the internucleoside linkages in the region may be, e.g., non-stereogenic. At least 60% of all the internucleoside linkages in the region may be, e.g., non-stereogenic. At least 70% of all the internucleoside linkages in the region may be, e.g., non-stereogenic. At least 80% of all the internucleoside linkages in the region may be, e.g., non-stereogenic. At least 90% of all the internucleoside linkages in the region may be, e.g., non-stereogenic. At least 50% of all the internucleoside linkages in the region may be, e.g., non-stereogenic. A non-stereogenic internucleoside linkage may be, e.g., a phosphate phosphodiester. In some embodiments, each non-stereogenic internucleoside linkage is a phosphate phosphodiester.

The first internucleoside linkage of the region may be, e.g., an S_{P} internucleoside linkage. The first internucleoside linkage of the region may be, e.g., an R_{P} internucleoside linkage. The second internucleoside linkage of the region may be, e.g., an S_{P} internucleoside linkage. The second internucleoside linkage of the region may be, e.g., an R_{P} internucleoside linkage. The third internucleoside linkage of the region may be, e.g., an S_{P} internucleoside linkage. The third internucleoside linkage of the region may be, e.g., an R_{P} internucleoside linkage. The fifth internucleoside linkage of the region may be, e.g., an S_{P} internucleoside linkage. The fifth internucleoside linkage of the region may be, e.g., an R_{P} internucleoside linkage. The seventh internucleoside linkage of the region may be, e.g., an S_{P} internucleoside linkage. The seventh internucleoside linkage of the region may be, e.g., an R_{P} internucleoside linkage. The eighth internucleoside linkage of the region may be, e.g., an S_{P} internucleoside linkage. The eighth internucleoside linkage of the region may be, e.g., an R_{P} internucleoside linkage. The ninth internucleoside linkage of the region may be, e.g., an S_{P} internucleoside linkage. The ninth internucleoside linkage of the region may be, e.g., an R_{P} internucleoside linkage. The eighteenth internucleoside linkage of the region may be, e.g., an S_{P} internucleoside linkage. The eighteenth internucleoside linkage of the region may be, e.g., an R_{P} internucleoside linkage. The nineteenth internucleoside linkage of the region may be, e.g., an S_{P} internucleoside linkage. The nineteenth internucleoside linkage of the region may be, e.g., an R_{P} internucleoside linkage. The twentieth internucleoside linkage of the region may be, e.g., an S_{P} internucleoside linkage. The twentieth internucleoside linkage of the region may be, e.g., an R_{P} internucleoside linkage.

The region may have a length of, e.g., at least 21 bases. The region may have a length of, e.g., 21 bases.

In some embodiments, each stereochemically enriched internucleoside linkage in an oligonucleotide is a phosphorothioate phosphodiester.

An oligonucleotide may have, e.g., at least 25% of its internucleoside linkages in S_{P} configuration. An oligonucleotide may have, e.g., at least 30% of its internucleoside linkages in S_{P} configuration. An oligonucleotide may have, e.g., at least 35% of its internucleoside linkages in S_{P} configuration. An oligonucleotide may have, e.g., at least 40% of its internucleoside linkages in S_{P} configuration. An oligonucleotide may have, e.g., at least 45% of its internucleoside linkages in S_{P} configuration. An oligonucleotide may have, e.g., at least 50% of its internucleoside linkages in S_{P} configuration. An oligonucleotide may have, e.g., at least 55% of its internucleoside linkages in S_{P} configuration. An oligonucleotide may have, e.g., at least 60% of its internucleoside linkages in S_{P} configuration. An oligonucleotide may have, e.g., at least 65% of its internucleoside linkages in S_{P} configuration. An oligonucleotide may have, e.g., at least 70% of its internucleoside linkages in S_{P} configuration. An oligonucleotide may have, e.g., at least 75% of its internucleoside linkages in S_{P} configuration. An oligonucleotide may have, e.g., at least 80% of its internucleoside linkages in S_{P} configuration. An oligonucleotide may have, e.g., at least 85% of its internucleoside linkages in S_{P} configuration. An oligonucleotide may have, e.g., at least 90% of its internucleoside linkages in S_{P} configuration.

An oligonucleotide may include, e.g., at least one phosphate phosphodiester and at least two consecutive modified internucleoside linkages. An oligonucleotide may include, e.g., at least one phosphate phosphodiester and at least two consecutive phosphorothioate triesters.

An oligonucleotide may be, e.g., a blockmer. An oligonucleotide may be, e.g., a stereoblockmer. An oligonucleotide may be, e.g., a P-modification blockmer. An oligonucleotide may be, e.g., a linkage blockmer.

An oligonucleotide may be, e.g., an altmer. An oligonucleotide may be, e.g., a stereoaltmer. An oligonucleotide may be, e.g., a P-modification altmer. An oligonucleotide may be, e.g., a linkage altmer.

An oligonucleotide may be, e.g., a unimer. An oligonucleotide may be, e.g., a stereounimer. An oligonucleotide may be, e.g., a P-modification unimer. An oligonucleotide may be, e.g., a linkage unimer.

An oligonucleotide may be, e.g., a skipmer.

Terminal Modifications

Oligonucleotides of the invention may include a terminal modification. The terminal modification is a 5′-terminal modification or a 3′-terminal modification.

The 5′ end of an oligonucleotide may be, e.g., hydroxyl, a hydrophobic moiety, 5′ cap, phosphate, diphosphate, triphosphate, phosphorothioate, diphosphorothioate, triphosphorothioate, phosphorodithioate, diphosphrodithioate, triphosphorodithioate, phosphonate, phosphoramidate, a cell penetrating peptide, an endosomal escape moiety, or a neutral organic polymer. An unmodified 5′-terminus is hydroxyl or phosphate. An oligonucleotide having a 5′ terminus other than 5′-hydroxyl or 5′-phosphate has a modified 5′ terminus.

The 3′ end of an oligonucleotide may be, e.g., hydroxyl, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer (e.g., polyethylene glycol). An unmodified 3′-terminus is hydroxyl or phosphate. An oligonucleotide having a 3′ terminus other than 3′-hydroxyl or 3′-phosphate has a modified 3′ terminus.

The terminal modification (e.g., 5′-terminal modification) may be, e.g., a hydrophobic moiety. Advantageously, an oligonucleotide including a hydrophobic moiety may exhibit superior cellular uptake, as compared to an oligonucleotide lacking the hydrophobic moiety. Oligonucleotides including a hydrophobic moiety may therefore be used in compositions that are substantially free of transfecting agents. A hydrophobic moiety is a monovalent group (e.g., a bile acid (e.g., cholic acid, taurocholic acid, deoxycholic acid, oleyl lithocholic acid, or oleoyl cholenic acid), glycolipid, phospholipid, sphingolipid, isoprenoid, vitamin, saturated fatty acid, unsaturated fatty acid, fatty acid ester, triglyceride, pyrene, porphyrine, texaphyrine, adamantine, acridine, biotin, coumarin, fluorescein, rhodamine, Texas-Red, digoxygenin, dimethoxytrityl, t-butydimethylsilyl, t-butyldiphenylsilyl, cyanine dye (e.g., Cy3 or Cy5), Hoechst 33258 dye, psoralen, or ibuprofen) covalently linked to the terminus of the oligonucleotide backbone (e.g., 5′-terminus). Non-limiting examples of the monovalent group include ergosterol, stigmasterol, p-sitosterol, campesterol, fucosterol, saringosterol, avenasterol, coprostanol, cholesterol, vitamin A, vitamin D, vitamin E, cardiolipin, and carotenoids. The linker connecting the monovalent group to the oligonucleotide may be an optionally substituted C₁₋₆₀ aliphatic (e.g., optionally substituted C₁₋₆₀ alkylene) or an optionally substituted C₂₋₆₀ heteroaliphatic (e.g., optionally substituted C₂₋₆₀ heteroalkylene), where the linker may be optionally interrupted with one, two, or three instances independently selected from the group consisting of an optionally substituted arylene, optionally substituted heterocyclylene, and optionally substituted cycloalkylene. The linker may be bonded to an oligonucleotide through, e.g., an oxygen atom attached to a 5′-terminal carbon atom, a 3′-terminal carbon atom, a 5′-terminal phosphate or phosphorothioate, a 3′-terminal phosphate or phosphorothioate, or an internucleoside linkage.

### V. Pharmaceutical Compositions

An oligonucleotide of the invention may be included in a pharmaceutical composition. A pharmaceutical composition typically includes a pharmaceutically acceptable diluent or carrier. A pharmaceutical composition may include (e.g., consist of), e.g., a sterile saline solution and an oligonucleotide of the invention. The sterile saline is typically a pharmaceutical grade saline. A pharmaceutical composition may include (e.g., consist of), e.g., sterile water and an oligonucleotide of the invention. The sterile water is typically a pharmaceutical grade water. A pharmaceutical composition may include (e.g., consist of), e.g., phosphate-buffered saline (PBS) and an oligonucleotide of the invention. The sterile PBS is typically a pharmaceutical grade PBS.

In certain embodiments, pharmaceutical compositions include one or more oligonucleotides and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligonucleotides may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions including an oligonucleotide encompass any pharmaceutically acceptable salts of the oligonucleotide, esters of the oligonucleotide, or salts of such esters. In certain embodiments, pharmaceutical compositions including an oligonucleotide, upon administration to a subject (e.g., a human), are capable of providing (directly or indirectly) the biologically active metabolite or residue thereof. Accordingly, for example, the disclosure is also drawn to pharmaceutically acceptable salts of oligonucleotides, prodrugs, pharmaceutically acceptable salts of such prodrugs, and other bioequivalents. Suitable pharmaceutically acceptable salts include, but are not limited to, sodium and potassium salts. In certain embodiments, prodrugs include one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligonucleotide, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions include a delivery system. Examples of delivery systems include, but are not limited to, liposomes and emulsions. Certain delivery systems are useful for preparing certain pharmaceutical compositions including those including hydrophobic compounds. In certain embodiments, certain organic solvents such as dimethylsulfoxide are used.

In certain embodiments, pharmaceutical compositions include one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions include a co-solvent system. Certain of such co-solvent systems include, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol including 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intraocular (e.g., intravitreal), intravenous, subcutaneous, intramuscular, intrathecal, intracerebroventricular, etc.). In certain of such embodiments, a pharmaceutical composition includes a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain.

### VI. Methods of the Invention

The invention provides methods of using oligonucleotides of the invention.

A method of the invention may be a method of inhibiting the production of an NRL protein in a cell including an NRL gene by contacting the cell with the oligonucleotide of the invention (e.g., a single-stranded oligonucleotide of the invention or a double-stranded oligonucleotide of the invention). The cell may be present in a subject (e.g., in a subject's eye). The cell may be a photoreceptor cell.

A method of the invention may be a method of treating a subject having a disease, disorder, or condition (e.g., retinitis pigmentosa) by administering to the subject a therapeutically effective amount of an oligonucleotide of the invention or a pharmaceutical composition of the invention. The diseases, disorders, and conditions that may be treated using methods of the invention include retinitis pigmentosa (e.g., Rho P23H-associated retinitis pigmentosa, PDE6-associated retinitis pigmentosa, MERTK-associated retinitis pigmentosa, BBS1-associated retinitis pigmentosa, Rho-associated retinitis pigmentosa, MRFP-associated retinitis pigmentosa, RLBP1-associated retinitis pigmentosa, RP1-associated retinitis pigmentosa, RPGR-X-linked retinitis pigmentosa, NR2E3-associated retinitis pigmentosa, or SPATA7-associated retinitis pigmentosa), Stargardt disease (e.g., ABCA4-associated Stargardt disease), cone-rod dystrophy (e.g., AIPL1-associated cone-rod dystrophy or RGRIP1-associated cone-rod dystrophy), Leber congenital amaurosis (e.g., AIPL1-associated Leber congenital amaurosis, GUCY2D-associated Leber congenital amaurosis, RD3-associated Leber congenital amaurosis, RPE65-associated Leber congenital amaurosis, or SPATA7-associated Leber congenital amaurosis), Bardet Biedl syndrome (e.g., BBS1-associated Bardet Biedl syndrome), macular dystrophy (e.g., BEST1-associated macular dystrophy), dry macular degeneration, geographic atrophy, atrophic age-related macular degeneration (AMD), advanced dry AMD, retinal dystrophy (e.g., CEP290-associated retinal dystrophy, CDH3-associated retinal dystrophy, CRB1-associated retinal dystrophy, or PRPH2-associated retinal dystrophy), choroideremia (e.g., CHM-associated choroideremia), Usher syndrome type 1 (e.g., MYO7A-associated Usher syndrome), retinoschisis (e.g., RS1-X-linked retinoschisis), Leber hereditary optic neuropathy (e.g., ND4-associated Lebe'rs hereditary optic neuropathy), and achromatopsia (e.g., CNGA3-associated achromatopsia or CNGB3-associated achromatopsia). Methods of the invention may be used to treat subjects having a disease, disorder, or condition associated with a dysfunction of ABCA4, AIPL1, BBS1, BEST1, CEP290, CDH3, CHM, CNGA3, CNGB3, CRB1, GUCY2D, MERTK, MRFP, MYO7A, ND4, NR2E3, PDE6, PRPH2, RD3, RHO, RLBP1, RP1, RPE65, RPGR, RPGRIP1, RS1, or SPATA7 gene. Advantageously, because the oligonucleotides of the invention target NR2E3 and not ABCA4, AIPL1, BBS1, BEST1, CEP290, CDH3, CHM, CNGA3, CNGB3, CRB1, GUCY2D, MERTK, MRFP, MYO7A, ND4, PDE6, PRPH2, RD3, RHO, RLBP1, RP1, RPE65, RPGR, RPGRIP1, RS1, or SPATA7, the therapeutic activity of the oligonucleotides of the invention does not depend on the type of the mutation responsible for the dysfunctional ABCA4, AIPL1, BBS1, BEST1, CEP290, CDH3, CHM, CNGA3, CNGB3, CRB1, GUCY2D, MERTK, MRFP, MYO7A, ND4, NR2E3, PDE6, PRPH2, RD3, RHO, RLBP1, RP1, RPE65, RPGR, RPGRIP1, RS1, or SPATA7 gene.

The oligonucleotide of the invention or the pharmaceutical composition of the invention may be administered to the subject using methods known in the art. For example, the oligonucleotide of the invention or the pharmaceutical composition of the invention may be administered topically to the eye of the subject. Additionally or alternatively, the oligonucleotide of the invention or the pharmaceutical composition of the invention may be administered to the subject intraocularly (e.g., intravitreally).

### VII. Preparation of Oligonucleotides

Oligonucleotides of the invention may be prepared using techniques and methods known in the art for the oligonucleotide synthesis. For example, oligonucleotides of the invention may be prepared using a phosphoramidite-based synthesis cycle. This synthesis cycle includes the steps of (1) de-blocking a 5′-protected nucleotide to produce a 5′-deblocked nucleotide, (2) coupling the 5′-deblocked nucleotide with a 5′-protected nucleoside phosphoramidite to produce nucleosides linked through a phosphite, (3) repeating steps (1) and (2) one or more times as needed, (4) capping the 5′-terminus, and (5) oxidation or sulfurization of internucleoside phosphites. The reagents and reaction conditions useful for the oligonucleotide synthesis are known in the art.

The oligonucleotides disclosed herein may be linked to solid support as a result of solid-phase synthesis. Cleavable solid supports that may be used with the oligonucleotides are known in the art. Non-limiting examples of the solid support include, e.g., controlled pore glass or macroporous polystyrene bonded to a strand through a cleavable linker (e.g., succinate-based linker) known in the art (e.g., UnyLinker™). An oligonucleotide linked to solid support may be removed from the solid support by cleaving the linker connecting an oligonucleotide and solid support.

The following examples are meant to illustrate the invention. They are not meant to limit the invention in any way.

### EXAMPLES

### Example 1. Inhibition of Target Nucleic Acid Expression In Vitro

Oligonucleotides may be assessed for their ability to knockdown a target NRL nucleic acid in a cultured cell line expressing high levels of the target NRL nucleic acid. Selected oligonucleotides may be incubated with a cultured cell line expressing high levels of the target NRL nucleic acid. Relative target NRL nucleic acid reduction may be determined using standard techniques useful for quantification of nucleic acids. For comparison, the measured target NRL nucleic acid levels may be normalized to target NRL nucleic acid levels in a cell treated with a negative control oligonucleotide. Alternatively, the measured target NRL nucleic acid levels may be normalized to housekeeping gene levels.

A positive control oligonucleotide may be transfected to ensure appropriate cell transfection efficacy. The transfection may be effected using a transfection agent, e.g., LIPOFECTAMINE.

Dose response analysis may be conducted in the selected cell line. Dose-responsive reduction in the target NRL nucleic acid levels indicates that an oligonucleotide is effective at reducing the expression of the target NRL nucleic acid.

### Example 2. Functional Testing of Oligonucleotides in Explanted Retinal Cells

An oligonucleotide may be tested in a physiologically relevant primary culture assay using, e.g., intact retinas from wt mice. In this assay, suppression of Rho expression may be used as a read out. After a culture period with media containing vehicle or an oligonucleotide, the retinas may be collected and assessed for Rho expression. Rho is a well-described target of NRL in rod photoreceptors. Oligonucleotides of the invention may cause a substantial reduction in the Rho expression compared to a vehicle in retinal explants from mice. NRL loss-of-function mutations typically lead to a reduction in rod gene expression. To determine whether the same was true for our oligos, explant cultures of murine retinas treated as described above may also be assayed for rod photoreceptor genes, e.g., NRL, NR2E3, GNAT1, PDE6A, PDE6B, RHO, GNB1, and CRX. After a culture period (e.g., after 2, 3, 4, 5, 6, or 7 days), an oligonucleotide may decrease the expression of the rod specific genes compared to vehicle treatment.

### Example 3. Rhodopsin Expression Reduction in the Retinas of Adult Mice

Oligonucleotides may be tested for their effect on adult photoreceptor gene expression in vivo. Oligonucleotide compositions may be administered intravitreally to one eye of an adult mouse (>P21). After a predetermined period of time (e.g., 1 week, 1 month, 3 months, and/or 6 months following the administration), expression of photoreceptor genes may be measured in the treated eye and in the untreated eye. The photoreceptor gene expressions in the treated eye may then be compared to those in the untreated eye. Treatment with oligonucleotides of the invention may reduce the expression of Rho and rod specific genes, e.g., NRL, NR2E3, GNAT1, PDE6A, PDE6B, GNB1, and CRX. The Rho and the other rod specific gene expressions may be assessed by qPCR (nucleic acid) and by Western blot (proteins) analyses. The oligonucleotides may also increase the expression of some cone photoreceptor genes (e.g., GNAT 2, PDE6C, GNB3, OPN 1SW, OPN 1MW, ARR3, and/or THRB) in the adult retinas.

### Example 4. Rod Degeneration in Mutant Rhodopsin Retinas

The effect of the oligonucleotides of the invention on Rho expression in adult rods may have potential as a way to slow the degeneration of these cells in dominant forms of retinitis pigmentosa, e.g., Rho P23H. In this disease, the affected individuals express a mutant form of rhodopsin that is likely inappropriately processed and ultimately leads to the death of the rods. Reducing the NRL expression using oligonucleotides of the invention may slow the degeneration of the rods.

The assay for assessing the effect of an oligonucleotide of the invention on retinitis pigmentosa may be performed as follows. Retina from RhoP23H transgenic mice at P8 may be explanted and maintained in media containing vehicle or an oligonucleotide of the invention. The majority of rod cell deaths in the RhoP23H transgenic line typically occurs between P14 and P21. Therefore, explants of retinas from RhoP23H mice at P12 were made and treated the explants with vehicle or an oligonucleotide of the invention. Here, designations P8, P12, P14, and P21 refer to the post-natal age of the test mice. In these tests, P8 explants allow for the assessment of the activity of the oligonucleotides of the invention in decreasing the level of expression of Rho, and P12 explants allow for the assessment of the activity of the oligonucleotides of the invention in preserving the cells in the outer nuclear layer (ONL).

After an extended culture period, the retinas may be subjected to histologic analysis. The number of nuclei may be counted in the outer nuclear layer (ONL) of each retina in the central region. Retinas treated with an oligonucleotide of the invention may have a greater number of rod photoreceptors in the ONL than vehicle-treated controls.

### Example 5. Rod Degeneration in Mutant RPE Retinas

Oligonucleotides of the invention may slow the degeneration of adult rod cells in recessive forms of retinitis pigmentosa, driven by mutations in genes like Phosphodiesterase 6 (PDE6). PDE6 is highly concentrated in the retina. It is most abundant in the internal membranes of retinal photoreceptors, where it reduces cytoplasmic levels of cyclic guanosine monophosphate (cGMP) in rod and cone outer segments in response to light. In this disease, the affected individuals express a mutant form of PDE6 that ultimately leads to the death of the rods and cones.

Oligonucleotides of the invention may be assayed to assess their effect on the degeneration of the photoreceptor cells as follows. Retina from rd10 mice, carrying a spontaneous PDE mutation, at P8 may be explanted and maintained in media containing vehicle or an oligonucleotide of the invention. The mutant rods may then be assayed for the rhodopsin expression levels, and the rhodopsin expression levels may be compared to those in the wild-type retina. Rod degeneration in these mice starts around P18. Therefore, explants of retinas from rd10 mice at P16 may be made. The explants may be treated with vehicle or an oligonucleotide of the invention. Here, designations P8, P16, and P18 refer to the post-natal age of the test mice. In these tests, P8 explants allow for the assessment of the activity of the oligonucleotides of the invention in decreasing the level of expression of RHO, and P16 explants allow for the assessment of the activity of the oligonucleotides of the invention in preserving the cells in the outer nuclear layer (ONL).

After an extended culture period, the retinas may be subjected to histologic analysis. The number of nuclei may be counted in the ONL of each retina in the central region. Retinas treated with an oligonucleotide of the invention may have a greater number of rod photoreceptors in the ONL than vehicle-treated controls.

The studies described herein demonstrate that the oligonucleotides of the invention may be useful in the treatment of multiple inherited retinal degenerations (IRDs) in a mutation independent manner. The inherited retinal degenerations include, e.g., diseases, disorders, and conditions associated with a of ABCA4, AIPL1, BBS1, BEST1, CEP290, CDH3, CHM, CNGA3, CNGB3, CRB1, GUCY2D, MERTK, MRFP, MYO7A, ND4, NRL, PDE6, PRPH2, RD3, RHO, RLBP1, RP1, RPE65, RPGR, RPGRIP1, RS1, or SPATA7 gene. Non-limiting examples of the diseases, disorders, and conditions that may be treated using oligonucleotides of the invention include retinitis pigmentosa, Stargardt disease, cone-rod dystrophy, Leber congenital amaurosis, Bardet Biedl syndrome, macular dystrophy, dry macular degeneration, geographic atrophy, atrophic age-related macular degeneration (AMD), advanced dry AMD, retinal dystrophy, choroideremia, Usher syndrome type 1, retinoschisis, Leber hereditary optic neuropathy, and achromatopsia.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. An oligonucleotide comprising a total of 12 to 50 interlinked nucleotides and having a nucleobase sequence comprising at least 6 contiguous nucleobases complementary to an equal-length portion within an NRL target nucleic acid.

2. The oligonucleotide of claim 1, wherein the oligonucleotide comprises at least one modified nucleobase.

3. The oligonucleotide of claim 1, wherein the oligonucleotide comprises at least one modified internucleoside linkage.

4. The oligonucleotide of claim 3, wherein the modified internucleoside linkage is a phosphorothioate linkage.

5. The oligonucleotide of claim 1, wherein the oligonucleotide comprises at least one modified sugar nucleoside.

6. The oligonucleotide of claim 1, wherein the oligonucleotide is a gapmer.

7. The oligonucleotide of claim 1, wherein the oligonucleotide is a morpholino oligomer.

8. The oligonucleotide of claim 1, wherein the oligonucleotide comprises a hydrophobic moiety covalently attached at a 5′-terminus, 3′-terminus, or internucleoside linkage of the oligonucleotide.

9. The oligonucleotide of claim 1, wherein the oligonucleotide comprises a region complementary to a coding sequence within the NRL target nucleic acid.

10. The oligonucleotide of claim 1, wherein the NRL target nucleic acid is NRL transcript 1, 2, 3, or 4.

11. The oligonucleotide of claim 1, wherein the oligonucleotide comprises a region complementary to a region within the sequence from position 547 to position 1260, position 354 to position 753, position 569 to position 634, position 807 to position 866, position 1149 to position 1260, or position 888 to position 911 in NRL transcript 1.

12. The oligonucleotide of claim 1, wherein the oligonucleotide comprises a nucleobase sequence comprising at least 6 contiguous nucleobases complementary to a region comprising a sequence selected from the group consisting of positions 642-645, 766-769, and 1127-1130 in NRL transcript 1.

13. The oligonucleotide of claim 1, wherein the oligonucleotide comprises a nucleobase sequence comprising at least 6 contiguous nucleobases complementary to a region comprising a sequence selected from the group consisting of positions 892-895, 974-977, 1175-1178, and 1235-1238 in NRL transcript 1.

14. The oligonucleotide of claim 1, wherein the oligonucleotide comprises a nucleobase sequence comprising at least 6 contiguous nucleobases complementary to a region comprising a sequence of positions 721-724 in NRL transcript 1.

15. The oligonucleotide of claim 1, wherein the oligonucleotide comprises a nucleobase sequence comprising at least 6 contiguous nucleobases complementary to a region comprising a sequence of positions 904-907 in NRL transcript 1.

16. The oligonucleotide of claim 1, wherein the oligonucleotide comprises a nucleobase sequence comprising at least 6 contiguous nucleobases complementary to a region comprising a sequence selected from the group consisting of positions 825-828, 933-936, and 1031-1034 in NRL transcript 1.

17. The oligonucleotide of claim 1, wherein the oligonucleotide comprises 8-24 contiguous nucleobases complementary to an equal-length portion within an NRL target nucleic acid.

18. A double-stranded oligonucleotide comprising the oligonucleotide of any one of claims 1 to 17 hybridized to a complementary nucleotide.

19. A double-stranded oligonucleotide comprising a passenger strand hybridized to a guide strand comprising a nucleobase sequence comprising at least 6 contiguous nucleobases complementary to an equal-length portion within a NRL target nucleic acid, wherein each of the passenger strand and the guide strand comprises a total of 12 to 50 interlinked nucleotides.

20. The oligonucleotide of claim 19, wherein the passenger strand comprises at least one modified nucleobase.

21. The oligonucleotide of claim 19, wherein the passenger strand comprises at least one modified internucleoside linkage.

22. The oligonucleotide of claim 21, wherein the modified internucleoside linkage is a phosphorothioate linkage.

23. The oligonucleotide of claim 19, wherein the passenger strand comprises at least one modified sugar nucleoside.

24. The oligonucleotide of claim 23, wherein at least one modified sugar nucleoside is a bridged nucleic acid.

25. The oligonucleotide of claim 19, wherein the passenger strand comprises a hydrophobic moiety covalently attached at a 5′-terminus, 3′-terminus, or internucleoside linkage of the passenger strand.

26. The oligonucleotide of claim 19, wherein the guide strand comprises at least one modified nucleobase.

27. The oligonucleotide of claim 19, wherein the guide strand comprises at least one modified internucleoside linkage.

28. The oligonucleotide of claim 27, wherein the modified internucleoside linkage is a phosphorothioate linkage.

29. The oligonucleotide of claim 19, wherein the guide strand comprises at least one modified sugar nucleoside.

30. The oligonucleotide of claim 19, wherein the guide strand comprises a hydrophobic moiety covalently attached at a 5′-terminus, 3′-terminus, or internucleoside linkage of the passenger strand.

31. The oligonucleotide of claim 19, wherein the guide strand comprises a region complementary to a coding sequence within the NRL target nucleic acid.

32. The oligonucleotide of claim 19, wherein the NRL target nucleic acid is NRL transcript 1, 2, 3, or 4.

33. The oligonucleotide of claim 19, wherein the guide strand comprises a sequence complementary to a sequence comprising positions 586-605 or 264-283 or 815-834 or 965-984 in NRL transcript 1.

34. The oligonucleotide of claim 19, wherein the hybridized oligonucleotide comprises at least one 3′-overhang.

35. The oligonucleotide of claim 19, wherein the hybridized oligonucleotide is a blunt or comprises two 3′-overhangs.

36. A pharmaceutical composition comprising the oligonucleotide of any one of claim 1 to 35 and a pharmaceutically acceptable excipient.

37. A method of inhibiting the production of an NRL protein in a cell comprising an NRL gene, the method comprising contacting the cell with the oligonucleotide of any one of claims 1 to 35.

38. The method of claim 37, wherein the cell is in a subject.

39. The method of claim 38, wherein the cell is in the subject's eye.

40. A method of treating a subject in need thereof, the method comprising administering to the subject a therapeutically effective amount of the oligonucleotide of any one of claims 1 to 35 or the pharmaceutical composition of claim 36.

41. The method of any one of claims 38 to 40, wherein the oligonucleotide or pharmaceutical composition is administered intraocularly or topically to the eye of the subject.

42. The method of any one of claims 38 to 41, wherein the subject is in need of a treatment for an ocular disease, disorder, or condition associated with a dysfunction of ABCA4, AIPL1, BBS1, BEST1, CEP290, CDH3, CHM, CNGA3, CNGB3, CRB1, GUCY2D, MERTK, MRFP, MYO7A, ND4, NR2E3, PDE6, PRPH2, RD3, RHO, RLBP1, RP1, RPE65, RPGR, RPGRIP1, RS1, or SPATA7 gene.

43. The method of any one of claims 39 to 42, wherein the subject is in need of a treatment for retinitis pigmentosa, Stargardt disease, cone-rod dystrophy, Leber congenital amaurosis, Bardet Biedl syndrome, macular dystrophy, dry macular degeneration, geographic atrophy, atrophic age-related macular degeneration (AMD), advanced dry AMD, retinal dystrophy, choroideremia, Usher syndrome type 1, retinoschisis, Leber hereditary optic neuropathy, and achromatopsia.

44. The method of claim 43, wherein the subject is in need of a treatment for retinitis pigmentosa.

45. The method of claim 44, wherein retinitis pigmentosa is Rho P23H-associated retinitis pigmentosa, PDE6-associated retinitis pigmentosa, MERTK-associated retinitis pigmentosa, BBS1-associated retinitis pigmentosa, Rho-associated retinitis pigmentosa, MRFP-associated retinitis pigmentosa, RLBP1-associated retinitis pigmentosa, RP1-associated retinitis pigmentosa, RPGR-X-linked retinitis pigmentosa, NR2E3-associated retinitis pigmentosa, or SPATA7-associated retinitis pigmentosa.


SEQUENCE LISTING (5 sequences)

SEQ ID NO: 1 - DNA, 43329 bp, Homo sapiens
tcttacaaaccttggttaataaggagcttgtcaaattcccacaccagtccgctttcatgccctcctgggcaactagaaccttagcggcctggctgtacagtcctgacttctggcccaccttgctgtcaatgaaggtgatgccatcctggtgtccagccagtgcacccacaggcttggggtcatcctcccgcatggtgcgtcgatcccacactttgcagatggcatcatctcccccagagaacaggatttgggagcttatatcagcaaaggccactgcattcacatcatcctcatgggactcaatctaggcgaaaaggaggccaggatataagacctctttctaggtcttataaaaagccaaaaaccaggaggcagaggctctatctcaggagccatacctgaagggtgcgccggttctgttctcggtcaaagacatacaggcagccatcattggccctgaaagggaagggataaaggagggtcctggagccatgatcgagaggaccaacgtggtatatgctctagagagttccactgagaaatcctgctctgggtcaagccctttgctctttttatacaggaagtgggggaagagttagctgtgtttcaaggaggcttgacacctccaatctcgtgctggggaaatggagagcaaggggtgaagcaacttgtaaatcctgaccttgggagaaggtctggttgttctctgcagctgccagtgccatctgtgtaacagcagaactcatctctggcagaacccccatcttgttattgagggaaacataccccacaagcacttaccctcctagtacttctcgtccatctgaggagacagcaatggagaagacagcaaagcgacgctcatctggcctaaaagcaagaaggagctgggttaagtggacttccttcagcttttgttaatctacataaaatctgacaacggttgtaaggcacatccccaggggaaaaaagaagtctggctaacatgcaccataatgcagatgctcagtgagaacccagaaatcatcctgcaaccatctattttacccagtcctctggctgacaagatggtgcttccctcccaggaatgcacacagacccaccagtatactttttcccctaaacagaacttccaactcaagaggtttgctcccaaagagcttcccttgagaagtttctgatgagtctgaccagaaagggaagccagtacctgagatccagggcagtgtgtgtatctccctcaccatagatattgcagatatgaactaagagaagcaagaaaaacaagaatctttcagggatgggggaagaagagtggaagtgaagctagggaagagatcaaacaggggacaagggtactggagcactttgggagtgattttcccaggacaaagtgagggatcctggctcagaggggacaggcacatggaaacctcaggtcccaggctggagagtcagtggaagcctggtgcatactcactgtaatcagaccagctagagtagaggaagtggttcccatcaggggtgaaggccacatccaagacgctccagcctacgtcgcgggccttgatgctcttgaatttacggaaacggccatatcggcagtcatagagtcggattgtctggtctgtgtaagtaaagatcaggcagaattatgggacagtttccttgtaacttttctagattagacatctcccactcagtgtacctgtggagtcttccctttatcatgggaaacatggtctggctaagcatccttaagccaggtctgagctccccgtctgtggctttctagcctcagtcagggggaatgtcccatgttccaggaaaagatagtttataggaccagggtctggtaccttggcaagcagacatgaatatttgaccatctttgctgtagatgccacagaaagccttctgagagtagctatcagtgaagcccagatcattgggcaagaagctagggaaggagagagaaagtgagggtgaggggtgctcaggataaggagtgtcccaatcagcagaatcctaacagagctggaatatcagaacagtcattcttcccttttggctgtgagtgcaatcccgtctttcaaccattccaccatcctctaaatttctacaattttctacacttttctactttgtttactctcaaagtttcaaccccatttaacctaacctttaatatctctgaggctcctttaacacctaccccttcaaagtttaggagataagacctatctggcccttagagtctcttcaccaagccccatactcactgagatatcactcgagactgttctccaagggagaagcttccccgatggcagaggccccgttctctctaaatggaagggattggggaatattgctagcagaaatagagggccacttcagccatatcaagccccctcctccaaagatattgctcttgttatagaaatggacacacacatcatagtctgtcccacagaactccgatggaggctggttaactctccagccccagcgcttgaggcttctactttggaaagttgacttcctttttctctggggctggcagagcttctggctgctttggcaggcctgctgccaggctgggaggtggagaggcctacctggtgcaacattcgaggaaagctgtgcttctgggcggcccgcctaagccccagctgccctgtggccagttccacttgtgtcttgatctcattgaattccagctcccgggtgtcaggggtagcatccactgtgagtaccaaggagaagtttcatcctaggtctgccagcctcagaggacagttagcagaactgtacccaagcccacccctgtggctgaatatactggggctgtaaggactccttattccttaaattatcgtaccttctttcaatgtaacctatcattcaaaatatcactaaaaggttttctagtaaaagtcttccaacattaggggcttttcctcttaccaggtgggttgtatcgatccccaagacgaccatcccaagctctgtcattctcttcctctgagtccaagagggcctgaatgaattgtaaatttgctgcacctcctccctgcaccaacctcacttggcctctgtggagagacccccattataaaggaggggacttcttcccccaaaccaaccaaattcagagtcaatggctatctagctcttagaccacttggtactcattttgaggtttgaggctgactagaccttggctgttatttttggtctcctataaaacacagtaggaaagagagttttactcttacagcaagagcaagaccaacatgctgtggacgtgacctactaagttgacctcccagcgagctaaacattctaacatcatttccttagaacaagagcaagaccaatacgctgtggacatgacctactaagttgacctcccaacaagctaaacatcctcacagcatttccttctgagcctctgcctgagaagtccaccacagaagacaatattctaagctgggtgcggtggtgtgtgcaactgtagtcttagctacctgggaggcaaggattgcttgagcccaggagttgcaggctgtggtgtgcacttgatcacacctgtgaatagcccgttctccagactggggaacacagtgagccctgtctatgtctactaaaaacaacacaaaacacaaacaaaaaagaatacaatattctgaagggacccccaaacccatcttctcctcaaagagccataagcaaagaaagaaagccagctgcacccttttccagtcaattccaggtggtctctcccttcaaggcattctttcctcaagaacaggttagagtcagggcaaattaaacttgtttttcaagtaaatcgaagtggcctcatatcatgtgtcaactgccttactaaaaagttcaacactcccttcccatctcttcaccccttacatcactcctttcccactaggaccccccccttttttttttcaagcttacttggaaaatagactatatttgtgtagtcacagagacaggctcccttgaatagcccctcaaacacatgagcaggtcctcttggtgaccctccctaggtaagaaccactcttactgggtatggagctgagtgccatgggtgcccagcagggaaaggctgttcagctcaaacagaacagctgtcctgagcgaatcccatttcctaacctctgtaattttcctgcctggagttgggatcaaggcactgtggtagcacctgctgggggtcacaccagagggtaagttacctgcggaggagataggccagtacctgggccagatccacatcttcatcctcttcttcctcttcctcactccgacgcaggccagcccctcttcggggcaagccctcggaggggtctccggacccggatcctgcactgctgctgttccgcgatcccatcttctggtcacagcattcttaggtcctgtgcgggggctcctcctgtcacctccttgggtttggtgaaagcaaagcgtgctctagaaataccagggtctcctgagacggggtagtagctatctctaggagggtgacccttcctcggggctgagatcccttttctttagcctatgccaactaccaaggcgtcgggaacacaaaccttctccaatcctgtgaagcaaagaaagcaagagtgaaccgtagtgcgctaccctagctcaccaactgaagaaagatttgttttctacaccaaactcacctatcgaagtccccggactggggagcgggaggggtgttgggcggtgttggatttccaggtaacctctctgaccgaccaatcgaaatccctctcggaagaaagcgcttcactagcttatctcatctaaccaattagaagccttaggactcaaagaccctcgacgcccccaccttatcgaccaatcagagcgcccccttacaaaggccggcagcagtgacagccaatgaaaatagaatttggcctaacacccctccttcctcgcctcacgcccacgtggtgcgccgggggtgtgtacgccatgagggggaggaagccgagaagcacctcccactgaagccggcggtaaccgccccacgcgagaccacccaccagccaggagcggtcgcaggacccgcagcggcgcgaggccttgtttacaccgactgccgccactgcgttggccaaccgctcctccgtcacgcgcttcgctcgtcacgcatctcgcggcttcctggccggcgggctgcaaacgtcacacgacggccccgcccccttggccagccaactggacagcggagcacgatgattgataggtaggcgggagaatgcgcgaggtgcctgaggggaggagaaagtgcacacccaaggagggctgttagctcttgctggggaggagtaagtgggaactgccagcttcctgaatcagagacagccgaactaggcgtaacggctgagaggcggggcttaaaaaagaaacgtttggtggttcgctaccgagaggtattttagcaagggatataatttactgtctgtccctaaagatcatttggcatcgcacttttcagacagtgcccaagggcctcctttctccccactgcttcccctgtccagctggagccgttcttctgtcgctggccgcagctccagtcggttctgccctaaggggtgcccagcgtcccagtagacctccataccgtccttccgggcttagcttttggaagccttgctcttgggacggtccccttttagacctccctttatccggtctgacaattttttaagacaatccctggctttccgccgcggtctttgcagacgttccctaatacacgcgtctttctgtcaaagcttttccggttccgctgtcccctttgacgctcccttggccctccgggatttgcagaccttcgctagtccctctgtcctcgcggtgcctctccgcccccggccgcgggtccagggtgcgcgctcgccgcctcctgccccctcctgccccctcctgcgggtttatgagtaacaagcctgagtccaaggccgggagttgccacatctctgatcctcgccctggctgggtggggacgtgtgggttgaatgaccttgctgtctcgggctcagaggctcagggtccgagctgaacttcattccctgcagcctgcacttccttgggaaagctgcgagcttccctccgcccgctcctgagggcagggatgggagcgagggctcgtggccttcttgggccctcctggggcctctcccagtgagggcgagggtttggccagctttcgggcgacgtctcctagtctctagatccggagacctgagcttgtcaccagggctagctttgatggcctctgcgcttagaagggtcctgaatgtttggtttaatgttctagtgttaacggtcttgaaattattagtaattttaaagcaagtgcctggtattttcatcttgcactcggctccactgattatgtaggcggtctgctgggcgcccatcctctattgccagattacttactatctgtgctttaacgtttattgagactttaggattccgtggtttctgggttatccagcctggaaaatcgggatctttatttattgattgattttacctgcgttctcccagaatgtgacggaatcattttttttttttattatactctaagttttagggtacatgtgcacattgtgcaggttagttacatatgtatacatgtgccatgctggtgcgctgcaccactaacgtgtcatctagcattaggtatatctcccaatgctatccctcccccctcccccgaccccaccacagtccccagagtgtgatattccccttcctgtgtccatgtgatctcattgttcaattcccacctatgagtgagaatatgcggtgtttggttttttgttcttgcgatagtttactgagaatgatggtttccaatttcatccatgtccctacaaaggacatgaactcatcattttttatggctgcatagtattccatggtgtatatgtgccacattttcttaatccagtctatcattgttggacatttgggttggttccaagtctttgctattgtgaatagtgccgcaataaacatacgtgtgcatgtgtctttatagcagcatgatttatagtcctttgggtatatacccagtaatgggatggctgggtcaaatggtatttctagttctagatccctgaggaatcgccacactgacttccacaatggttgaactagtttacagtcccaccaacagtgtaaaagtgttcctatttctccacatcctctccagcacctgttgtttcctgactttttaatgattgccattctaactggtgtgagatgatatctcatagtggttttgatttgcatttctctgatggccagtgatgatgagcatttcttcatgtgttttttggctgcataaatgtctgcttttgagaagtgtctgttcatgtccttcgcccactttttgatggggttgtttgtttttttcttgtaaatttgtttgagttcattgtagattctggatattagccctttgtcagatgagtaggttgcgaaaattttctcccatgttgtaggttgcctgttcactctgatggtagtttcttttgctgtgcagaagctctttagtttaattagatcccatttgtcatcctccctaactcattttatgaggccagcatcattctgataccaaagccgggcagagacacaaccaaaaaagagaattttagaccaatatccttgatgaacattgatgcaaaaatcctcaataaaatactggcaaaccgaatccagcagcacatcaaaaagcttatcgaccatgatcaagtgggcttcatccctgggatgcaaggctggttcaatatacgcaaatcaataaatgtaatccagcatataaacagagccaaagacaaaaaccacatgattatctcaatagatgcagaaaaagcctttgacaaaattcaacaacccttcatgctaaaaactctcaataaattaggtattgatgggacgtatttcaaaataataagagctatctatgacaaacccacagccaatatcatactgaatgggcaaaaactggaagcattccctttgaaaactggcacaagacagggatgccctctctcaccgctcctattcaacatagtgttggaagttctggccagggcaatcaggcaggagaaggaaataaagggtattcaattaggaaaagaggaagtcaaattgtccctgtttgcagacgacatgattgtttatctagaaaaccccatcgtctcagcccaaaatctccttaagctgataagcaacttcagcaaagtctcaggacggaatcattttttaaaaaaattggccgggcgcggtggctcacgcctgtaatcccagcactttgggaggccaaggcgggtggatcacctgaggtcaggagttcgagaccagcctggccaacatggtgaaaccccgtcactaaaaaaaaaaaagccgagtgtggtggcatgcgcttgtaatcccagccactcgagaggctgaggcaagagaatcgcttgaacccgggaggtggaggttgcattgagccgagatcacgccactgcactccagcctgggctacaaaagcgaaactccgtcacacacacacaaaaaacaatcaaccaaccaaacaaaaaacacctatgcctcttccggtagttgatctgctgaagaatcctcctctaacagcatttagtgcttaacgcacaagtgagctgggcacggtggcttacgactgtaattccagcactgtgggaggccgaggaggaggatcatttgtggccaggagttcaaggccagcctgggcaacatagtgagacccagtctacaaaaagaaaaaaaaaatagccggccatcgtggcgcgtgcctatggtcccaacttacttgggaaggatgaaggagggaatcttgagcctatgaggtggaggctgcagtgagccatgattgctccactgtactccagcctggggatagagctaaagactgtctctaaaagaaaatcaacaaatggctcactattgttcattgtttcaaatacaaacttccaaacctggatgtctatttttagtaacctcattttttgatatttctgctaagcaaatatgccaataaatagcttgtttatttttacctctgcaccttcccaggagaatttcttattagagggaagctgtctacaaaactcccttcctcacagccttgcactttgacccagtaatactgcttccagaaatctaggccggacatgcataaaaatttatgttctacatagcaattattgataaaagtggaacactggaaacaaagatctaaatatcttgcaatatgggattgttaaataaattatggtccatgtagtatagacacagaaataccaggtagctattaaaatggtgttgtagtcttcaatttttttttaaggtacctattcctggtataggtttgttgtagccttctatttttataccataggaaattgtgcaaactacattgtcaagttaaaaaaataaagataatcatttcttctggtgacatgtaatagatgcaaaaacgatgtggtttcattttttcattagaaagtataataggcgtggtagctcacacctgtaatcccagtactttgagaggctgaggcgggcagctcgcttgaccacaggaatttgagatcagcctgggcaatgtgacgaaaccccatctctacgaaaaatacaaaaacaaaattgagctgggcctgtagtcttggctacccaggatgctgaggtggaaggatcacctgagtctggtcaaggctgcagtgagccatgattgcgccactgcacttcatccagcctgggcgagaatgagaccccgtctcaaagaaaaaaaaaaaaagtataataatacccagctcattacatcattaaacaaagtggggagggcttgcatgaactggtgacaaaggtgtagcttctacaaaggattatgatgaatacagctatagcacctgaggtccagaaaaacgaacaaaaaacttctgttaaacttatctccttaacaattcattttttgattctcttttctcttcttcttacaaattagtttaaacctgaataaaatcttttaattcaagcttttgtaatatggaacttgtacattattgagtaaaacatttaaaagtcatggtaaatgttaaaataactgaggttttatgacaaaattgagtccttcacatcacatgggaaagaatataagattaatcactggattcattgattaatttttttgcgtataaaagattttttttttttttttttgagatggagtcttgctctgtcacccaggctggagtgcagtggaccgatctcggctcactgcaagctccgcctcccgggttcatgctattctcctgcctcagcctcccgagtacctgggactacaggcgcccgccaccatgcccggctaattttttgtatttttcttagtagagacggggtttcaccatgttagccaggatggtctcgatctcctgacctcatgatctgaccacctaggcctcccaaagtgctgggattacaggtgtgagccaccacgcctggcagcatataaaagatttttaagcctatgaacttgattatctcactatgacctgtaatgtttttcaaaatgtgtaagagcatttatatcttagggtatacagtgtgtctattgaaataaataaaatggcatatctttaaaaataattatggtgaagtcattttgatagactaaattttgcctatgctgtcttgattttgaaatttggtacgttgcgtggttacagtttgtgaagcacttttcatttgggtaacctctactctctccagttgcacttaaagtcacttaaagccacagagaggtttttctaccttcagcacccattttaggacaatttctggtaattgctaagtaaatgtcagctagttgacttcctgcctgacctgtcttggcctgtttcctgctcattagcatctccactagagggcaggctgggctgctgaggagagaaggaaaggagtgttttcattctctagctaaccctgtcctcctctcctctactgcagctcccagcttggtgaccacacagagctcctgggacagttccgtgtttggagtgactgatgctcatacatgtgtttgaacattgccttatagtattctctgtgtcgatggaaatggagagtgtgctaataatttacctagggacttaaaatgtgttgactagaaagccactaagtaactatgggatgttctaatactctcatgtcttgtgtctgtaagaatcagatttttctgtgtgtagggtaggagctatagatgtaatataagtaaataccctttagtggcccggggtagtggctcacacctgtaatcccagcactttgagaggctgaggcaggtggattgcttgaaaccaggagttcaagaccagcctgggcaacatagtgagaccccatctctaaaaaaaaaaaaaaaattagccaggtgtggtggcttacacctgtagctactcgggaggctgaggcaagaggatcatttgagcccaggaattcaaggctgcagtgagctgtgatcacgctactgcattacagcctgggcaacaaagcaagaccatgtttccagaaaaccctttagccctgattttgaatagaacatgtcagtatgagttcatgaggttttatcttctgtcgctatccaccaaaaagatctagaaacaatgaccaatccagtatcaatgaacactcctagcatccagattgtggtcttaaaacgccatttccccctgaaagaagccagagcttcttagagaaaaagctgattccaggtttggggaaggaaatgaacacaatgagcatggagcaacttataccagatagccgggaagcaaggagtatgtatcatgtcaaaaggactcagctctttttggcctagcttccatagccaaatgtgggacaatttgaacatttaataaggtcaatttaataaacactaagtatgtttaaatccatgagttcataatgtttgtgtgtaactaactggttaccttttcaaaggataataggaaatcaaatcatttttttgaagatgataaataaagataaagaattaagcagttatcctgctgtttctatataaacaataccatagggtaatgaaatagaagtgtccattttataaaattattgcaatgttatgacaactcaaatatggctgttttgcaatccctagtgagttaatgcatttagacactgaacatctgagtctggattcgtcaatgggtaaattgaaggaaaagaaaggaatggagggggaatctgtagactgatagagacttgaaagacatatgaaaaaaattttaaatgggagcaaagctaaactatagtgtctaaggatctacatttaggtgatcaaactataaaaaaatgcaaagaagtaattattactcaccatagtatccactatggcaggaaggggtggctatgatagagatgggcaacagggtcggggcggggcggggctgggttggtgtctgcaaagttctgtttctttccctgggtagtgttccaaaggtgttcactttatgatagtttattcagccataactttgttttagatggtcttctgtatctgtgttttattttaaaataaaaagattataatgtgttaactatattttatcagttatctggagctgaagggcatgcagccccattttattctaattaataaaaataatttttaaggctaacactttcttgtccctgtgcatttagaagataccagaaaggtgcctggaaccccagttccctgcctcttagctggagggatttatttttaaatctattttctttctctcctagattttatttacattgctcatgttccttttaaactgtagagtcagcaatatgtaatatacgcttacagggcctactctaatcattataaagtggaaaggtgatgatagctaacatgtattaaatggttattcggtgccaggcatagttctgagtgctttattccactgactcttttaatcttcccaacaagactgtaaagtgtaggcatttccattttacagatgaagcaactgagaggcacctctggctcaagtctactatgtgtttaatacctgtgctatgttgcttttgacggtattcagttatcatatgctatttatgaagctaaggatttggctgtgttttattattatttttaaatttttaaatttatttatttatttattttttttttgagacagagtctcactctgtcgcctaggctggagtgcagtggtgtgatctcagctcactgcaacctcagcctcccagtttcaagtgattctcccgcctcagcttcctgagtagctggaattacaggcatgggccaccatgcctggctaatttttgtatttttagtagagatggggtttcaccatgttggtctagctggtctcaaactgctgacctcaggtgatctgcccacctcggcctcccaaagtgtgagccaccgcgcctggctggctgtgttttaaagtacaatatactccactgctaactttgttctaacctctgttccactctgacctttagatttttacacattctggctggtcactggcatttcctatgagaacacttggtgtttcctcttctagttttttcctttgagggagttcccaataattccattttatttgttgaagtttgttccctttttctgggtaaagactaccacactacccaccttaggttatacattctctcagcctgatccagattgcacagaagttatcctaagctgatgcccctgactcatgtgagatcattgcactgttttcctgattactctccaaagtttatttcagatgaatttaggctactctaaactgtagttcgcagaccggcagcatcagcattacccaggagcttgtaaggaaaacaaaatcttgaggctactacagacttgctgagtgacaatctgcattttaacaagatctccaggtgattcaaaggcacttttaagtttgagaaacacggaataggtgactttttatttttttatttttttgagacagagtcttgctctgtcccccaggctggagtgcaatggcatgatctaggcttactgcaacctctgcctcctgagttcaagtgattgtcctaaatagctgggattacaggcacctgccatcatgcccagctaatttttgtatttttgtagagaaggggtttcaccgtgttggccacgctggtcttgaactcccaacctcaggtgatccgccctccttggcctcccaaagtgctgggattataggcatgagccactgcgcccggtggaataggtgacttctaaggtccttgcagttgtagatgtctatactactatgaaagacatgtctttgcagttttatcactttaacccttagctctattgtttactaactgtgaagctttggacaaattacttgacctctctgtgcctttgttacttcatctgtaaaatggtaattcccccaaagggttgttaggtatgtgcttagaacactgccttgttgggaataggcccccaaaatctggccataaactggccccaaaactggccataagcaaaatctctgcagcactgtgacatgtttgtgatggccatgacacccacactggaaggttgtgggttcaccggaatgagggcaaggaacacatggcccaccctgggcagaaaaccgcttaaaggtattcttaaaccataaacaatagcatgagcgatctgtgccttaaggacatgctcctgctgcagataaccagccagagcccatctcttcattttggcccatccctttgtttcccgtaaggaatacttttagttaatctataaaaacaatgcttatcactgacttgctgtcaataaacacgtgggtaaatctctgttcgaggctgtcagctctgaaggctgtgagacccctgatttcccactccacacctctatatttctgtgtgcgtgtttttaattcctctagcactgctgggttagggtctccttgacagagctggtctcagcagtgcctggcataaagaaggtgccacagaagtgttgttagctgtcctaatattgaatatgcttccatgaatactatgcagctcccagtgccaggtcaggcagggggaagaacaaattcgagaactgacaatgaagccacttcactggagaatggagttgtagaggcttctgcaatctaagtgggagatcaggaattttaggcttgggggctgtactgagcagcttgctgaggccctcagttttttttttgttttgttttgttttgttttttttttttgtgacggagtttcgctctgttgcccagactggagtgtgcagtggcacgaccttggtttactgcaacctctgcctcctgggttcacaccattctcctgcctcagcttcccaaatagctaggactacaggcgcccgccaccactcccggttaattttttttttttttttgtatttttagtagcgatagggtttcaccgtgttagcccggatggtctcaatcccctgacctcgtgatctgtgcgcctcagcctcccaaagtgctgggattacaggcgtgaaccaccacacccagcctcaccatttttttttttttaagaactcaggacatagagtggatagtgccttctcccagcaattcctatgtctgtgacccagtgctagagctacctgtaaaacagatctttgattgtgccaggctgtgcttcttctaggggctggaagaagatgattgtgagacaggtgttagagggaagcctgtgaagataggtgggaaatgaaaacagcacaaatcttgtgtaaaatagataagcatctcttattattggacagtctttgtgggaaggtctatggcacattgaagatgttagttaatatcaaattgctcatattttctgcaaggtgccttccctattcccagatgagggtgctatgtcctcttgctagactagggcctcaggtcacattttgtgcacacgtctctccagggcctcaagctcagccaacacctctttgggcagatcctggttgacctgctctgtcaggtagctccgaatgtcacgaacctcctgttcccagaagtccttggggagggagaacagctgagtggtgtctatagctctgaggccgctgagatccaaggctccttcctttggcaccagcccaatgggtgtctctcgggcactgtcctccccctctaaccgccggcagatccagtctagcacccgagcattctccccaaagcctggccacaggaagtgccctgcctcgtcacgccggaaccagttgacatggaagatacggggcagctgggcccccttgcgcccttccatgctcagccagtgttccaggtagtgcccgaagttgtagccaaaaaagggccgcatggcaaatgggtcgtgcatgatgatcttccctgggggggaagcaagatgggtaaggaatcttccttctcactggggtagggaagggcaccctgcatccagttggaggaagctgtgaaaggcttatggttctgaatatcttcagggatggggaaaagatagaagtgggaagaggtggggccatatcagactccaggggtggacaaaaggaaggtctggctcaggaagggagagaggacgtgctgtgtgcacacacaggagagggaggaatggtgagggtgctcacctttgtgttctgctgcagcagtggactcagagcgcatggcgctgcccacaaacaccccatgacgccagttgaaggcctcgtataccaggggtacccctagatcaccaatgaacagagtcacaggcagcccttttctttggaccctaactccagcccttctggtgagcttgactggtactctctctcctaagacctcggctatcccattttcagaccctttgcctcctcccaagtttgcattcaccaggggagaactctggagaaaggtgagatccaggctgaggctctggatcagagggagggaggtaggccctaatacagagatagcccttttgccaagaacatggagctcatatgttgtttacctttgggtctgcggccaccaaagatgatggcgtcaatggggacaccctctggggcctcccaggctgggtccatgatggggcactggcgagccggggcacaaaatcgagagttgggatgtgcacagggctccttgtcacctggcagaggaaatacaaacactattatttccaaggtcatgtcgacccccaacacacacatacatgggcacacattcttgcagggttctttttttttttttgcctcatcagctgcctagaaccttgtgtgataaggagcagagagaggggagccagggcatctccaagagctcaggttgtttagccatgaaggtgcggccagctgagctatgtgagaaaagagtgggagaggaattggggtttggcagggcactaatgtttgtgccaagctactggcacttggcactgaccagggctcaagggaatggggttagatatgggcataaggttataataatttttcccccagaaactatttcattatttgtgagattattatttaaatgaatacccaaacctaaagcaaaaactgagaatgtcagcttagtacttcagacaattgactttccgtcagcaaaccggggaagacaggttctcctaagttatccaaatgcaaataggaatttattttatctttttatttttgaggcagagtcttgctctgttgcccaggctgtagtgcagtggtgcaatcttggcttactgcaacctccgcctcccaggttcaaacaattctcctacctcagcctccccagtagctgggattacaggcacccaccaccatgcctggctgatttttttgtatttttaatagagatggggtttcaccatgttggccaggctggtttcaaactcctgaccttaactgatctgcccaccttggcctcccaaagtagtaggattacaggcatgagccactgcgcctggcctgattttatttacttaattaatttagttatgagacggagtctcactctgtcaccaggctggagtgcagtggcatgatcttgtctcactgcaacctctgcctcctgggatcaagcaattttcctgcctcctgagtagctgggattacaggcatgagccaccatgcctggctaatttttgtattttattggagacggggttttgccatgttgccaggctggtctcgaactcctgacctcaggtgatccaactgcctcggcctcccaaagtgctgggattacagatgtgagtcactgtgccctgcaaggaattgattttaaagctaacaacatgttggtgatttccctgccctgcttagaccctgatgaagtgatagggctggaaaaatagagatagggatatcacatgggtagggataacctcaggagcccaggtcacacttggcaccttcaggtgtcctggaggggggttgggactaagtcagtggcacatgtatgaagcaccactgtgtggcccctgagtggacatggtgccttctctgctggaggttgctggtcagacctggggagtggggaagccacggggccaaaatccccaccctctcccatccgccagcttctgcccgccattgcagataagagccatccttcttcagcacccagtgcagaatcctaacacagggtattattctgggaattgaagctggggacaagatgccctagttatgcccccaaatctggagatgatgaaagggttggggaactagcctgtggggaaagatgaaagtttcttggatttctcagttgggtgggaaggctgaggggggcttaatgatggtgttagtaaaaataaagggtgattagatgaagcaaggagggtgactgctaggccagcctggcaaacctgaatataggcttcagtgactacaaggccagcctcccctcccatgctcttctttgcccctgcctcttggtgtctggactgcctgactgcagaaggccatggaaagaatactgaaggtccagagtgcagggctgagggagaggtgggactcccaggcgcccagggtaatgacaggattcagaaactagcttctgggaaagactcaagcactgacctgattttgtttgtaggaaggattggggtgaatgctttggagggacaggggaagggtgacggtaacaatactgacctttagacagtggttattatacaggggacagtgggagccaagtggcaggaggagagttggagcagaggaggcagctttcctcttttcctaatgggccaaactttatggatagtgcttggagcccaataccttctgtaagaatgttcaaaagtccttccttttcctttcattaaaaaaaaatccagcctgtatttattattgagaatttactgtgtatttgtcagtgtgataaaccctttttatgcactatttaatcctccaacaatctcatgagataggtactatcacaactaccctcattcaactgatgtggaaactaaagcttagggaagttgagcaacttagccagatcacaaaaccagtgactggtggagttgggacatgaactgagattttgcctgggactggaaactgcaaacgttcatgacttctgtgcctcctggcagtcctagaagatggttggaggttgtggagaaagcttttccaccattaaggtgctgaggcctggaggctgtgccacaccttccccaccgcacataccaggtttccagggtttgcccagccaggaggtcacagtaacaccaggtggaagaggctggtcaatgccctcccagtacacgccaccatcactggtctcagccacattggtaaaaatagtgttactctggattgtagccatggcgttgggattggtggtggcagaggtaccaggggcaaccccaaagaagccgttctcagggttgatggcccggagtcgacctgttaggagaagtgaaagaaaggaaggaccaaacaaggaaaacttgagatgtaagaccaaagaaggtcctgctccacccacccctgtcctgagatggacccaagatctgatgaggaaaggctgtctctcgggtggctgggctgccagaggaatggagcagggttgggtgttcaggaaactttctcactgacaaagtcattgtgaacactagagaggagaccccgaggctctgacaagagccagaaccaagagtatgatctgagagtccctcaccttcactgtcaaacctcatccaagcaatatcatcccccacacactccactttccagcctggcagtgcaggccgcatcatagccaggttggtcttgccacaggcactagggaaggcggctgccacatagcgcttcttccctgcagggctggtgatgcccaggatctgtgcattggggagagagggaataagaaagaggtcaccaaaagtcagggcatggtctgcatggggaagggcagctagggtctaatagggaggaccaatgtttattaactatataagtgattgccctgccatcaggcctgggttagtggcaggggaaacctcaacattcagcagcctgaggccaaagctttgggccttcaagtagagtccccgcccaccagcctggcacctggcaggagggaggtgaggctgaggcagactggccaggccccacccctgccctgtccccggcagctgccctgcttctcaccaggccctcaccagcatgtgctctgccagccagccctcatcccgggccagccgagaggcgatgcgtagggcaaagcacttcttgcccagcagggagttgccaccatagccgctgccgaaggagatgatctcccgctggtcgggcacgtggccaatcagggttttctctgggttgcacggccactggctcactggctcccctggggacaatggggtcatggggctgctggcagcagcatcaggaactggggtgtcggggccagagggatcagggattggcaggagagacacttgggagcagtagcagcaccaagaggtggctgaagccccccacctcataataaccatcagcttccatgcccctattcttggacacggatttttctcaacctagtgcaaagtgctctgcaaacacagccttatccctctgctgggactgctgctctggatcttgggaattaggttgccagagaggacaggttcttagggtctgtggagaggtaggatttgggatttcctctgagtacaagaaggcctctgtgttccccttggggcagagcaggtgcttaccttgtcctgtcaggggctggcccacggagtgcagacacttgacaaagtcaccatctcccagggcctgaagcacaggtgtccccagtcgggtcataatacgcatgcttgccaccacataggctgagtcagtgagctgcaccccgatgcgggacagcggggagcccacaggacccatgctgaatggaagcacatacatggtgcggcctgtggaggaaggcatcccctggatgaagggttccaaacctgttcacatcttccgtgcccttgccactgtgcccctgccctggttaccctgcatgcagcctggaaacctctcatccacagctcgctggaaatcagctggggacatccagttgcccagctgcccacgggccccaccaggcgggagtggtaccgtgtcccgctgagaaggagttacaatcaccgtcttgctctctactcgtgccacatccttggggtctgtgcgggccagccagctgggggagaggggattgtcaggaagatggtggccagcaggtttcccttgtccctagacccactttttcaagtccaactttgactccaggtcttctgttggccttatgtttccccacccaatcacatctcagtcccttcctccaggctttgtcccatctaaggggtgttccccaaggtcccagccctctgggggtctctgcccctggcaaaagctccctgaactggtgaggaaaaaaaaaaaaagcaaaggatcttatttatatcagcttcttcatttaacattaaaataaaatacaaactacaaactaggctgggcgcagtggctcatgcctgtaatcccagcactttgggaggctgaggtgggtggatcacttgaggttaggagtttgagaccaacctggccaacatggtgaaaccctgtctctactaaaaatacaaaaatcagctgggtgtggtggcgcatgcttgtaatcccagctactccggaggctgaggcaggagaattgcttgagacaattccccggaggcagaggttgttgtgagctgaaatcatgccactgcactccagcctgggtgatacagtgagactccatctcaaaaaaaaaaaaaaatcccagtttctttcttttttttttttgagacagagtttcgctcttattgcccaggctggagtgcaatgacgcgatcttggctcactgcaacctccgcctcttgggtttaagtgattctcctgcctcaccctcccaagtagctgggattacaggcatgcgctgccatgctgggctaatttttgtatttttttttttttttttcagtagagacggggtttctccatgttggtcaggctggtcccaaactcccgacacctggtgatccacccgccttggcctctcaaagtgctgggattataggcgtaagcaaatcccagtttctgggtctgtttaccccaaagctctcattcttgttcattccattgggatgttctagttaagttgattttgccttggtgaacccaaagtggcctcagtgcacctatcctgcaggttgtggagcccaaggcttaccagttattgtacttggggagctttcggatgaggccctgctgctccagcagggtcagtgtggcagtattctcagcctcagttccatcacagatgtggatgccctctggttggcacaggcgggcactgtgctctacaaaatctcgaatgccagtgggaagctggcccagatctccactaagcactcgcagggtctggatgctacggcatgatggccagcccaaggggctcagcccatgccagttaagcctataggagaaacagaggcaatgagctagttgctgtcatcgagagtggacaaggcaggtggtggagagacagaaagcttgggctgcagcctgctaaaacatgtctgcatgcagggaccatgaagagcaggttgcagttggtgtgtgggatgtgttcaggacacatgtatcttgtagcctttatgaggcttgaggagggtgtctcctgctcagcgagggacagatatggccaaatgagcaaaaaaggcacatgcctggctgtgtggaagtatacatgtaggcatgctgatgttactgcctgtgtagcccatgcccatgtttaactgtttaatcaacgtttagaaataagtagggagatggctgggcacggtggctcatgcctgtaatcccagcactttgggaggctgaggcaggtggatcatgaggtcaggagatcaagaccatactgggcaacatggtgaaaccccatctactaaaaatataaaaattagccggatgtggtggcgtgtgcctctagtcccagctactcaggaggctgaggcaggagaatcgcttgaacccgggaggtggaggttgcagtgagccgagatcgggacactgcactccaacctgggcgacagagcaagactctgtctcaaaaaaaaaaaaaaaaaaaaaaaaaaaagaaatgagtaggggcctttggcccaagtgtcaagacaagaagttgggcccaggtgacaatttcatatgtctggtatttgaagcctcatccattttatttccattttcaagtatagttttttataaattaaaatggcagctgtgtttattctttccttctggtgtgtatgctgtaagactatgaaagccccaagcaaaggtcacagtctttccagtgccaggtttctagatgagggctcttgcacagagaataggtgactggatgctgggcttccaggccctgatctgctgctgcccacttggaccaacaatggttgcataaaatgttgccctcgggtaagtatttctgaactctgcggaaggttgaccactggggaatgagtgtcactggctggggttcatgggcttaattctcaggaggtcagcgtcctgttgtctgtcctctcatggccctgggagacagagaagcaacgtctgctctgattggcctggactcaaggtggagtaaacccgagagagcaaagaccatgaaggggatgttactctcctcctctatcaaggcctcgcccatcagaaactgcccccacacccaggaccacgtgaccccgtgcttcccctggctcttcttcctagaggccacatgcctagagagctttggtgttggtgggagggtggaacagcagcttgctggcagctcagactcagagggtgttagcctggggaaggaagaaagctcagagcatggatacgggagcttgagtctgtctggggtagaagctggggtggtttccgctaaagcaggatagatgatcagtgtggccccaggggcaggggaggagggctactgggacctgggtctgttcctgcctcagagggaagttctccgtgagcatcagttttcaagcacacagtcttgaggacagcctctctctccctggagcctggctcacgttatctcttgtgaagcccccacaccctcttgcagccctctaagtgggacgtttccttcagagaccacccacaagggctgcccaagtggaacgggctgtgcgacagatgtgggtcacttccgactctgggacaaaagctgagaagagaggcctcagagaataaataggagactaagaggacaggcagacaaacaggacgagcctgggggctccaggcctggacggaggaagaagccagctatgtaagaaacgaaggcaacttggcgccctcctcccaccttgcttgcctccctcccctgggcccaggtccctcgggctgctctggacttcccgccaggttccagtccttccagtccgggagaggccgggaatggatatttaaccttccaccttctttagggggctctgagcttagtctcccggggctcacgtacccttgtccccagggccctgctgcctctcgaagtaccctggggcgctggctgagagcccgttagaggtccgagggaggtatctgggaaggaaaggaggatatgcggagacctggctcagcagagaggagcgaggagagagaggaagttctgagagtggcagatagagatcgcgcggtcagaggcgagcgaggcagagaaggggaggtgcaggcggaaacgaggagcaccggccggctgccctcccaagagacgcgcggcgcgggaggctggacccacagcagtcgcccctgcccgcctccgcctaggatgggaaacctggcggaggctgagagcaggagagcgccactggcagccccgagggggcgagcgcagcggaaggtgcgggtgggccccgggccgggggtcactcaccgcaggccagggcggtacaatgcggccatggcacctgggcaggggctgcggggtggccaggaaccgagcggagccggggaggtatggaaggcggggaaggagggagcgcggcgaagcgagccacagcagaggctggcgggaggcgcttaaaaaggaggggggcaggcgcgggggcggaacctggtagggctggcactagctttcctccgccccgctggcaggccagctccctcggccccggggctccaggctccaggtcagcccagccgccgccccaacccctgcctccaaaccgccaaacccatctctcccattggaggactgggtccactcttcggcccccgacttcgaggccccgccccccatccgccaccgccctcccagccgcacatgatgtaactttgaggactcttgctcccgggagagacactaggtggcaaacaagctaggaacgaggggcgaggccaggggcgggagactacaggggccagacgtctagaagttagggggttgggcattgcctttccagttcctcctcctggggacgcgacaccctgagtcctgggcctttctgtagtccaagtgcccaatgctaaggtccacttagtccccaactcctaaatccccgtttctcctcctgatcaaggctcctttctgccttgccatgaaatggtggagtctctctacccctgggtctcttccttggtcctctgatctggtcattctcctcccccggcacatcccctcttcccctgacggagtcttactctgtcacccaggctggagtgcgtgcagtggcgcgatctcggctcactgcacactccgcctcccgggttcaagcaattctcctgtctcaacctgcagagtagctggaattacaggcgcctgccaccacactcggctgattttttgtattttttgtaaagacggggtttctccatgctggccaagctggtctccaactcctgacctcgtgatccgcctgccttggcctcccaaagtgctggaatttacaggcgtgagccaccacgcccggcctggtcattctttctctccacaatccaatgaggaaactcatctgcccacccaactaccatcataaagtcctcaggtgatttctcaagctatacctccattgggacatctctctccaacctgagaccactggatatcacccacaatgctatatagcacctcaaactcagcatatctatgatgggtgtctgctcagcatccactcgccttttctggtaatagcaccctgattttcttttggggaaccatctcctcagtctgtattgtcatgtgggtctgatgtcacccctcttgggggtaggcttgcagcttagccaacttttgtgttcctcactctggccacagcaatcaaggatgttcaaggatgggcatgagatccctctctgggactttttctaaaactttggaagttagctctctttctgctgagttgctgagctggtagaatataagcctggagctgctgagtgttagcttggcactgcaaagcaggaacctggctgagacagaagccagcctagaggaagcagagcagggagatggcgagcaatggattcctgatttcagttacaggaattgatgcatttcttctcttgctcaagctaattttaattggagttctgccacttgcaacagaaacagtcttgattctcagatttcttggagttctatgctggaacatagtgggataaggagagctggtctccagccacgggcttgctactggtccccgtctcttccccctcaggctctagcctacactcaacctcaggtatatgtatttggatctctttccccccagttcatagatccaaactctgtccacacttacccagctgcaaacacatacctcttggccaacctttgggcctaaaggtgtgcacactggtggcagcatggttgacctgaaggtcatcatcttctccataatcctgcacctgctctcatggtttcctacctcagcaaatggcaccatcatctggccagttgccccaaagggaatcagtctagactattcttatcctgcatagctaatcagatgccaggacctttggaattgtacttctcaattagccctcaagtctgcaactatccccatggccagggccttagttcagggcttatctctcacctggcttcaggtgatagcttctaaggcacttcttggactcctgcctctccaaacaccaacccctctccctaccaagccactccccacactgtgcatgcaaatccacttggaaagttaagagagaagaataatgtttatgctatggacagtaggtgccaggctctgtgtcagcaactctgcatgcataatctcattctgtttcacaactatattttacatatgtggacacgggcttaaagagtcatttgctcggtcaccctaagggaagaatcaaggaaaaagggaggcaggaccccagaagtatgccaacatataacaccctaaatcaaaggtcaaatgccgcacttgacctccaagatgcctgcttgggcctcttccaagtgtactttcctttctttcattcctgctctaaagctttttaataaacttccattcctgttctgaaaaaaaattattcatgaatattttatacttttatcctattatgaatgaaatgttctaattgtttaccacaagtacatacaaataaaattgctttgtgtatattgaaaaaaaaaagagtcatttgctcaaagtcacacaaatggttagcaggagaaatggaatctgaatccatatctttctgactctaattaccaagtccccctctccctataaatcctatactgtccccttgatttaccattaaaaattagatcatatcactcttaagcttaaaatctcttccaggctctttccatcacatgatatcttatgtccttcataatctgactcctatttatctaatgttgtcacccccacatccccacttgcctccttttctcctacgtactgaactctgaagtttccagagcacttctgtgaactttttctgggcatctgcactcaaattaaacacacaccatcctggccaacatggtgaaaccccgtctctactaaaaatacaaaaattagcttggtgtggtggcgcacgccggtagtcccagctactcaggaggctgaggcaggagaatcgcttgaacccgggaggcggaggttgcagtgagccgagatcgcaccactgcactccagcctgggcaacacagtgagactctgtctctaacacacacacacacacacacacacacacacacacacacacacacacaggccttttctgttcgtcccttagaatgaacatctatttcatctgtgttgcttctatcccatgtatatgcctatatttttgtactttactatactgtagttacttttatatctctgtaatcctcactagagtgatgatttcagtcttgtttttctcctagttcccagcagtacctggcctataacaggcacttaataaaagttggttgaataaaagaatgaatgagtgccacgtttctgaattcttcttccccaccattactttcttctctagctttaggcagatggagcaaggggctcacgtgcagatttccttgcactgctcatttccccttatctgccacacacttttctttagttccctgccgggagagcctggcccaccagctggccctatgtccaaggagtaccaccagggggcagagtgtgatctgcattgtcctctctggggcctctgggttcatccctgaagtctcacagcccgagtagtgatgctgtggtgtgtagcccagattcccgcttcaggatcatgatgctcattgttccagctgctgagtgttggctgctgatagctcacagctgagtacttccttgagcactatctgtggccaaagggagctgtctcaccaaatgcaatgcccgcttcctgggggcagtatgtatccaatgaatggttgttataaagtacagaggcctagccctttgtctcagtttgggacaactctgaaggtccatcccagccccagagctctttaggctcagctgattacaactgcagtgccattcaactgttccctctcttgggtcctgctactctttctttgaaagtccccccccgtgccccccccccccccccgagtaaaccacctgcacacaagtttctgtctcagagtgtgcttccagttgcaaatattcctcaggctcaacttggttacggcattagtcctctaatctgactcttatttcattgttgccttcctccacaaacacaaagtgctgacatacgtgatccttccgaacacaaatctgatcacatcatgtctctactgaaaacccttcagtagcttctccctgctcttagaataaagcccaaacccttaatgtgacctcccaggctctgcatgatctggcccctgctgacttctccagcctcagctatcactgttcttcccaccttccaggctttctactctctggctacgtggatcttttctcagttttgtgaacatgtctacatctttcctgcctcacaacctttgcatatgttgtttctgccgcctggaattcccaccctgtccacacctccaggcagggtcacttcttattcacctgaccagtcttggtttagcatgactttcttaggcaacctgtccctgtgatgcatcccctctggctttgtattagagtatgtcttcgtggaagaaaatttcatagcaccctgcccttcttcttcttagcacattcatctctgtttaaatggtttgtttttatgtagacacatctctaacatatgtaggacctgcggcaggagtacaaatagaggccctcaaaccttgtgtttaaatatttaaaatgggctgggtgtggtggctcatgccttgtaatcccagcactttgggaggcagaggctggaggatcacttgagcccaggagtttaagaccagcctgaacaacatagtgagacttcttctctacaaaaaataaaaaagtagccaggcatggtggtgtgcacctgtagtcccaactgcttgggagactaaagtgggagatagcttgagcccaggagttcaaggctgcagtgagctatgattgcaccactgcactccagcctgggtaacagagcgagaccctgtctcaaaaaaaaaagattaaaatgtataactcagggtcggcgcagtggctcatgcctataaccccagcgctttgggaggctgaggcgggtggatcatttgaggtcaggagttcaagaccagcctggccaacatgaaaccctgtctctactaaaaatacaaaaattaggccaggcgcagtggctcacgcctgtaatcccagcactttgggaggccgaggcgggcggatcacgaggtcaggagatcgagaccatcttggctaacccagtgaaaccccatctctactaaaaatacaaaaaaaaaattagccaggcatggtggcgggtgcctgtagttccagccggagtctgaggcaggagaatggcatgaacctgggaggcggagcttgccgtcagccaagatcgcgccactgccctccagcctgggggacagagcaagactctgtgaaaaaaaaaaaaaaaaaaaaaattagccaggcatggtggtgcatgtctgctacttgataggctgagccaggagaatcacttgaacccaggagacagaggttgcagtaagccgagatcacaccactgcactccagccagggcgacagagctatctcaaaaaaaaaaaaaaaaaagtataattcaggctaacaaactgttacacaaaatatattctgttcttctaccttgacaaatatattgacaaatacatacttcacaatggcctggaaggccaggttcagatttgtattttatttggaatcctcagggttctgcaccagaatgtgatggtgtagggggatgtagttttggccactggccctcagctgggcccactttgcctcctacccctgacaccatcttggactgcaaggggccttgtgtggaaccatgtggacctcccatcaggccatcccaagtctgtccacagtctcctgcaagcagctaccccttgtctaccctttgacctcagaatgtgtgaaccagtcatgtggcccagccgtgggagggcaaacccagggaagaggttcacgtgggccctaggagtgggcttgggccttttggacagggaatgcttagctatctggagtgtggtcctgagaggattaaggtatgggcagaggatagttggactcctcacccatgtggtgggacatagccagaggaggccagagtggagccttctaaagcacagggcccagggcaggggccactcctgcctgggcctaaggacaataatttatttattattttttttttgagacagggtttctttctgtctccaaagctagagtgcagtggcaccatcatggcccactgcaccctcatcctccccaggctcctgatcctcccacctcagtttttgtatttttagtagagacagggttttgccatgttgcccaggctggtgtcgaacttctgggctcaagcgatccacccatcttggccttccaaagtgctaggattacaggcatgagcctctgcacccagccaaagggcaataatttgatgtttctctgtttcatcagtctgtaagtttcttgaagtcagggctggtattagtgtaagttactgaggggtcgctaatgccttactcaggatgtggtgcagaataggtgtttaatatgcatttttcaaaagaatgtaggaggtacaccaagaagaggttgaggatttggatctattactgaagatctagataccatgagccagaagtaggaatgagaaccagtctttcagggttccaaaaatgagagtgcaggacagaggagaggaggtagcatcctggaatgctgactgtctcattttctctccttcctcttcattttctaccccactcctgcaggccctttgcctgcctgcccctctttccccatggtccaatatcactccatccatatcttgctacctttctgtcctttggccactgctcttcctctgtggcctatgtgtctatttctttgtattggtgtctaggtcttggtccccaatcttatgtcctgtcccttccttctaccactcttactgttctagtccttgttgtcccctactcccttcttggtcctctcctggaagacttcctttctttatccatctcgtccatgtgcctacatctttctgtctcctttaatccaaatccccatggagaccctgacttgtcctgtcctctatctcacatctacacctactgcctccttagtctccttcctctgttctattattcctggagtcacactggcttcatctttttcatccctagatgtccttgagcccattagtctcagtattcatttctcagtctccagtggatatgggagatgggctctgctttctgggtccccactgactgccacagactctcctctctgtgcccttggtcctttcctctggcccagactgggcccaaccctcactcccccagctcctttccctggtttagtggtgcctggtctgagccccaagcagctcaactcgcctgagcttgtttagtctgactcccttagcccgttagcagcttagccctcccacctctcctttgagccctccagtaaacaagaggatcagtgcagacctgacagctgctggttgggggcaagtgtctggccaggccagagcaggcatgaaaaaggggctgcactcaacacttcccattccttcttcaaccagggaaattgtagagtctctttcctgggtgtccactttggagagctacattccatgggcatgtgtgtggggtcctgcctctccatgatcctcattcccattttgtggttggttcctaggccttaggcttttttattcttttgagaaaacagcagtagatccaggcagtcacacagagaagcttgaatggtgacgatggtttctgctccttaaaacagaaacccaggtgtgggaagagagtgatgtcatctgatctaggttttccaatatgtgtatggaaagcatggtaggagagaccacatacacatgcacagacagacatatatacatgcctccccagggaagctgggtagactgcaggcattctgtgcagttctcaaactttctttcttttctcttcttttttttttgagatggagtctctctctgttgcccaagctggagtgcaggggcacgatctcggctcactgcaacctccgcctcccaggttcaagtgattctcctgcctcagtaggagtagctggcattacaggtgcctgccactatgcccagctaatttttgtatttttagtagagatggggtttcaccatgttggccaggctggtcttgaactcctgacctcaagtgatccgcccaccttggcctcccaaaatgctgggattacaggcgtgcctggcctttaaactttcaagcatgtcagacttgctggaagggccttaaaacacagatttctgggccccacccccggagtttctgactcagtagatttaaggtgagatcaggtaacatgtatttctagcaagttcccaggtgatatggatgctgctgctctggggaccacacttcagaaaccaccttcttagataatctgtatctgccagcttttccccaacatataaggctgggaccaggttaagttttataagtgatgacctttggggtctgatggacacccaggcctctgtgttgagaaagaggaactattaaccccatgggagtatttgggtggcctcagaaggtaggaagtggcctttaactccatagaccctatttaaacagcttcggacaggtttaaacatctccttggataattcctagtatccctgttcccactcctactcagggatgatagctctaagaggtgttaggggattaggctgaaaatgtaggtcacccctcagccatctgggaactagaatgagtgagagaggagagaggggcagagacacacacagggaaaaggtgagagaaagcagagggcatatgaggccagttacaaaagtgtctccgacggactggtggtgattagatttcaatgtcccttgaggcccagaaggaaggaacagagttccctgttctcttttttcctactctttctcccaagctggattagcaagaatgggagagacaaaatatggtggtcaagtcctggagatcccctgtgggaaaagttgctttcctttctgatgcgacggctatgaggggcataagaccagtcagcatgaagatggggcagaggtagaaatgtgaggattcttgaaaataggaaacagatccaaaaggcccggtgagagtgggcgtgggagagcttatagtctccaaagacagatgaggggctgcagggccttggaaggtgggggtgctgggagtttgtctggtcctctttttccaatcccaggctatgcatggctagggctgagggttgtgttcacatagaggggtggtggttcagacccatgtttctgtccgaactgggaaactgagcacttgggagtcggatcatcagtgaggagctttgttggggaagatgctccctttccttctttgagaataagcacacttgtactctcctctctctccatttcccccaaccccaggccaataagatatgcaaatgatctccttaccttattggttggaggcctcaggaccacagatgacctcagagagctggccctttaagaatgccctttgggctctgtgcccacagccccctggagctgagcagaggcaccaggccctgctccatggagccttcagtctcctgggaagctgtgcctgtctggctctggcactgaccacatcctctcggccatttctgaagtgagtatatccagggcctacctgggtgtcaggccgctcacctagaaccccctctttaacacctgctctctgttgtctgttttcaagaatcctaacaccttggtaccctactccactcccaccctgactggcctgtgtccctcactgctatcacgtcagaaagataaacaaccttccccactactccttctgtacatccagttctttctggatacctctctcagtgtctcaccatctcctttctgccaagacccagttcctttaatctgtgtctgtttccatcccctctgtcctctcaagggtgacaggttccatagagagggcagcaaccctgccctggaacaggaagagagaggcctgggtcttcctctttgcctctctgggtagagcctgggggtctgacatgtggttggaaagaaggctacctaggcttggggcatctagaaggagccgaagtctaagccctggggatgaacactccatgccgcgcaccttctgggggtggggtgggagttactggaattgggagctggactagagcttggacttcaaggctgggaagggtatctgaggtcacccagccagaagttcctcaggtgaacatgtgccccatgattgataacagacaggagagttccttggtgacataagtttaggacactttttgttgaacttgaacagacggtttactaaaggattgctcaggttaatatgctcttgtgctatgtgcatctgtaagaggtagggataggaggcaacatttctcaaagttatctgagcacagacctcttcataggtattgttcagaatcagtgttctagagaacactctttggtatatgttacaaagtctaactgccctcattgaacagaagggaaaacgggtcagaagggtgaaggtgatctgccaagttcacacaacgtaacaagttctgaatagaaggaaaatcaatcttggtctgttccccttggagagactggccttgaggaaagatggtggccagttgattctgatctttctagaactaattctaggacctttcttttttccatataaagcctcttgcccctacaaaagggattcattgattgattaatccatagaacaaatgtacactgagggtcttctataggcaaggcacttccctgagtgctttgagagacagagagctataagaacacattatccttggctcttaaaattcacagcatacatgccttcccactgtcctaaaatatacactcactttgtcacagttaattaagcaatttaaaaaaatggcttggaaatggaatgatgcctcttgagatgacagacctctcggcatgtctcagcactagggttgggagcactgccatactgctcttgcttttcatagatcctgaggcatcagtggggcgagaggctgtgctgtcctcttcctccttcaggaattcagctgcttgtcccctgtcaggagcccctgccctctgaaaggttactcttcagcctggtggggactctgcagtgaacagagctgcaccatccctctggctttcccaaactcttgctccagggcacttgggctttgagggaagagggacttggtgaagaggggatggcaggtggcctccatgtgctccagacctctcctcctctttgcaggtgcactcctcccagcccagctccagaatggccctgccccccagccccctggccatggaatatgtcaatgactttgacttgatgaagtttgaggtaaagcgggaaccctctgagggccgacctggcccccctacagcctcactgggctccacaccttacagctcagtgcctccttcacccaccttcagtgaaccaggcatggtgggggcaaccgagggcacccggccaggcctggaggagctgtactggctggctaccctgcagcagcagctgggggctggggaggcattggggctgagtcctgaagaggccatggagctgctgcagggtcagggcccagtccctgttgatgggccccatggctactacccagggagcccagaggagacaggagcccagcacgtccaggtgagtggtcagcaagctggcctgaggggaggcagggcaaggaaggaggactgcccaagagaggaaggggagctcccagagggggttatggactgggacaggggacagagggcaggagaaggggagaaggtcccttgaaagcaatcagatcgagaaaactacattctgcttctcccccttttcttaaaatggagagaaatgagtaggctgaaccaggaggagcagggagaagataagattagcagagaatccaaagggaagaattgagctggggagtgggcgactccggggggtaaacagatacatggtgtgtggaaaccagggaaagggttatgtgtggagtggagctgggttaagactggtttagattggccttttcaagacttcgtgcttcccagcccccaaccttctcaggaaggattgggactcatcctattaattacagacacagatgggggtgttgcgggcattaggatgtaatccaaatcctgtgagggtcaccgggttgcaggcttcaggacagggaaacgcagcgcgttggagggttgggggctgtctgggtgcgggtccactggacacacccgggcctggagctggatgccggggatcccagagacgagcccggggtttaggtgcgcgacgggctcgcctgaccgtggccggccctgcaccgtggggcgcccgcctgactggagcaacggtcagctggggggcccggggagcgtcggggcctggggcgggctctggaccgaaacagactgcgtggaagggcgagccttccggtgaaggtgggagccggggcggggctgtcccggggcggagccaggtagcgtcgggccctcagggcagagccgggtgcgacctggcgctgacccggtttctgcattctccctccgcagctggcagagcggttttccgacgcggcgctggtctcgatgtctgtgcgggagctaaaccggcagctgcggggctgcgggcgcgacgaggcgctgcggctgaagcagaggcgccgcacgctgaagaaccgcggctacgcgcaggcctgtcgctccaagcggctgcagcagcggcgcgggctggaggccgagcgcgcccgcctggccgcccagctggacgcgctgcgggccgaggtggcccgcctggcccgggagcgcgatctctacaaggctcgctgtgaccggctaacctcgagcggccccgggtccggggacccctcccacctcttcctctgagccgttcagagcaccttgtggtgtagtgggggctgggtggggtggctccgcccaggaggcggctgcacggttctctgcatcgttaccagagcgccttctggtcctagccacgccctgtatgaccgcgcaaatatccccaaagcttttgggtcctcaagtcatgcccgaatttagatgctggtcattttctggagaggggtcccctccccttacgaacacagaaacccagcccacatgactagcacgctgagctctgcagggaccagtgccaggcactggggggtggaagtgtggtgacacagtgaatgggaggtggaggagggttgcagctcccacctcagtttagtttttaattcagggttttcaacctgtaacacattaaagctgtaattagcaatgaggctgtattttcattctgaagcttgtaacctccccattttagcactacagaattttcaagatttcaatatccaacaactagatagattaggacctctatccgagatgctttttccctgcccaaccctgtggccttcagggctcagagcagcaaaggcctgaagagtgagctctgggggttgttggtgtgggttgggagagagctgtgtgcagaagtctggaaacctgggtcctagtcccagctcttccatgggatccccctgtcaccctgagcaaatcagttgcttcctggacttgtgttacttcatctaattctcatgtggattggacgacttctgctccctttccagttctggcatctccccagtatggaagtcccggtggtctccccaagaagtccccaagacaatctcgccaaaggcacctcctatcctgctgcagtttcccagctgcagcctaggcaggggatgcacagcccaggcgaggaagcctggcttctctgtgagcacatacgtgggtcctcggcagctccctccaggctgtctgggcctccagacctgcacagggtgctcctgccacctcccacctctctgagggctgaggtgagacttctcctgggatgacaatttgctgagagagtgcagcttttgtgaattaaacttgaagtccaggcagaattctaatgcaataagctaaatgttcttgcaatttaagaagtgttcattctttatccctgcttcaggctactgtttgtgtgtctgtgtgtgggagagggagagagagggagagagagagagagcaagaaaaagggaagtccccacctggagctgggtggcacatctgttatggagagccaatggaaatggggtggaggggcaggcattggaacagatggatcctccccctctcaccaccaccaccaccaaactcagaagcctcagtggcccagctcggcctgtcagtggctatccaggtctcacagtttccatggtaacccagtcctccagttcagcaggctaaggggagggcagatggggcctctgggcagagcacaggcgttgtgctgggagcagccgcctcacgagttactgtcatctgtcctctaccggacataataaatgggagaagctttcgggcatgggggaagaggctcccatatgtccctcttcctttctaccccctctctccaccccctccccagctgtctcttctcttcccatcacagttcttgctccctctttcccctcctctatcccagctcatccctcctccgcttaggagcctgcaatgggagtcctctcctccatcgctctcagaatttctctcgtctcctctcccttccccaccattggccactggccatcctctctggctctggattctctctctgaggggctcacagaacctttagcttctctttcttcatccccttccacactcattgttgaagggagaggatggggcagggagggtccttccgggagcccagctctttttctctcactctaaatttcacctgtgtctatccttgcctgccctggagctgtaattcagacccactacttttcttgttcacttttctcacctgcatggcacacaaagacagacagaaccatccatagacacctgagcacacaactaggccacatatacagtaccatacctgggtctcaaaatgatactcctgggttacctgagtatacttaaaaaaacaaagttgcacactgtggtgattaagagtagtgctttaggtggggcgcagtggttcctgcctgtaatcccaacactttgggaggccaaggaggaagtttgagaccagcctgggcaacatagcgagaaccctgtcactacaaaaaaataaaataaaataaattcagatgtaatggcatgcacctggagtcccacctacccaggaggctaaggtggaaggataacttgagcccaggagttcggggatgcagtgagccataatcgtgccactgcattccagcctgggtggcagagtgagaccctgtttcaagttgcagttctaccagttttttgaccatgggtgggttaaaatcaagcctcacttgattgtcagaaaaatggtgaaaataatggtaaccacctactgagaataaatgcatttaaataagcacttagcataaagttgacacatgctacgtaatgatatttgctgttgttgctgatgtgattgttaagtgtgtgaatctgtgtgttcatgcactgcacatccctgagtccatgagtgtgacccattaaggccatgaacagacacacacattgacacacccgggaaaccagactgtataggccttagatgctccactatgttcccaattcactagcagacctgctgccctccttcagtatcccccagcacctgctcttggcccgccccacagctgtgtgggggctggggttcagctcctgggggactcagcagggcctctctggagacagctataagaaagcttcagcctgccctcctgacagcccgtaattatcctcctatctcctgtaattattctccatggtgcctagaccccctggggttgggaggtgagggagggggctcctcagatcccactggcacatagctggggtaagactgcagagacagcagcctgcctggggagctctgtctcagattttgaaaggtgctggtcctcatttcttatggtgtgtgtgggggtgctgcaggctgcccagtgggcatctgaggcatgatggttagaggctgaggtgctaggagcagttctctttattaatggctagaaagtcaggatcacccaaggaagtcactgaggggccacagcattgaagggtatggggtttggagagataggagcaggacccaccactcacgtccagaacccagggggcacacctggtccaagaggtggaggcattggtcactggagtcacgagggtcaggacaggcacctgggagggggtgggagagtcataagcactctcttctacactacacccagcctagttgggctctaccaagccccactttgcaccccctgtgtccactcctttggaagccccaggactcactgagaggctgagggagtgtcggtccggagggaggcagtcacgggctagggctgggagtcgtagccagtgtgcagggcctgggagccccagggctgatgccctggctggcgtagtactccaccacctgccgtggcacctcagccaggacacacttggcgagtgcagagggggcagcctagggtggggacagtgatgtggttaggggtgagaggaagagtaggggaggcagggtcatctgagggagaagtggggcttcagggtggccagctgggctcccgcgtccttagcaggacatcaaatggtggaagacgaaggtcagatttggggggatgagaagctgaggagtccttcagccggaaggggcccgggggactcacatccttgaagtctcggaagggcacgaactggacaatgtctcgggctgcaggcacccctcgggggcagcgcaaggggccgtcgtcgccatccagcagccgcatgtcagagaagtcagcattgcccacgcctacgatgatgatggacatgggcaggcgggaggcacgcacgatagcagtgcgagtctcagccatgtcgctcaccacaccgtcagtgagcaccagcagcaccgagtacttctgagggcaagcagagagggtggaagggttggctttgtgtgcaagggcgttgacaccgtttccaccaaggtgcagaccctggccctggcaccacttaaagacacatgggacagctcctgtttgcccgccatgtcttcctaccgtggcttggccggtgctctgctcccgctgggccggctcagccacacggttgatgatgggggccacattggtggggccgtagagctggatctggggcaggcaacgacggtaggaggcgatgacccctgagatctctgtgagtgaagagaagttgtgaagctggcagaaatgagcagggccccctcctgacactccttaaagcaaggaaatgcttccaggtctgaggactgccctcccctcgagttccttcattaagcagtgggagagttggatggtcccttggcagggagagttgggtgcagatgctgggcctcggccctggaagcc

SEQ ID NO: 2 - RNA, 3810 nt, Homo sapiens
cgccgggggtgtgtacgccatgagggggaggaagccgagaagcacctcccactgaagccggcggtaaccgccccacgcgagaccacccaccagccaggagcggtcgcaggacccgcagcggcgcgaggccttgtttacaccgactgccgccactgcgttggccaaccgctcctccgtcacgcgcttcgctcgtcacgcatctcgcggcttcctggccggcgggctgcaaacgtcacacgacggccccgcccccttggccagccaactggacagcggagcacgatgattgataggccaataagatatgcaaatgatctccttaccttattggttggaggcctcaggaccacagatgacctcagagagctggccctttaagaatgccctttgggctctgtgcccacagccccctggagctgagcagaggcaccaggccctgctccatggagccttcagtctcctgggaagctgtgcctgtctggctctggcactgaccacatcctctcggccatttctgaagtgcactcctcccagcccagctccagaatggccctgccccccagccccctggccatggaatatgtcaatgactttgacttgatgaagtttgaggtaaagcgggaaccctctgagggccgacctggcccccctacagcctcactgggctccacaccttacagctcagtgcctccttcacccaccttcagtgaaccaggcatggtgggggcaaccgagggcacccggccaggcctggaggagctgtactggctggctaccctgcagcagcagctgggggctggggaggcattggggctgagtcctgaagaggccatggagctgctgcagggtcagggcccagtccctgttgatgggccccatggctactacccagggagcccagaggagacaggagcccagcacgtccagctggcagagcggttttccgacgcggcgctggtctcgatgtctgtgcgggagctaaaccggcagctgcggggctgcgggcgcgacgaggcgctgcggctgaagcagaggcgccgcacgctgaagaaccgcggctacgcgcaggcctgtcgctccaagcggctgcagcagcggcgcgggctggaggccgagcgcgcccgcctggccgcccagctggacgcgctgcgggccgaggtggcccgcctggcccgggagcgcgatctctacaaggctcgctgtgaccggctaacctcgagcggccccgggtccggggacccctcccacctcttcctctgagccgttcagagcaccttgtggtgtagtgggggctgggtggggtggctccgcccaggaggcggctgcacggttctctgcatcgttaccagagcgccttctggtcctagccacgccctgtatgaccgcgcaaatatccccaaagcttttgggtcctcaagtcatgcccgaatttagatgctggtcattttctggagaggggtcccctccccttacgaacacagaaacccagcccacatgactagcacgctgagctctgcagggaccagtgccaggcactggggggtggaagtgtggtgacacagtgaatgggaggtggaggagggttgcagctcccacctcagtttagtttttaattcagggttttcaacctgtaacacattaaagctgtaattagcaatgaggctgtattttcattctgaagcttgtaacctccccattttagcactacagaattttcaagatttcaatatccaacaactagatagattaggacctctatccgagatgctttttccctgcccaaccctgtggccttcagggctcagagcagcaaaggcctgaagagtgagctctgggggttgttggtgtgggttgggagagagctgtgtgcagaagtctggaaacctgggtcctagtcccagctcttccatgggatccccctgtcaccctgagcaaatcagttgcttcctggacttgtgttacttcatctaattctcatgtggattggacgacttctgctccctttccagttctggcatctccccagtatggaagtcccggtggtctccccaagaagtccccaagacaatctcgccaaaggcacctcctatcctgctgcagtttcccagctgcagcctaggcaggggatgcacagcccaggcgaggaagcctggcttctctgtgagcacatacgtgggtcctcggcagctccctccaggctgtctgggcctccagacctgcacagggtgctcctgccacctcccacctctctgagggctgaggtgagacttctcctgggatgacaatttgctgagagagtgcagcttttgtgaattaaacttgaagtccaggcagaattctaatgcaataagctaaatgttcttgcaatttaagaagtgttcattctttatccctgcttcaggctactgtttgtgtgtctgtgtgtgggagagggagagagagggagagagagagagagcaagaaaaagggaagtccccacctggagctgggtggcacatctgttatggagagccaatggaaatggggtggaggggcaggcattggaacagatggatcctccccctctcaccaccaccaccaccaaactcagaagcctcagtggcccagctcggcctgtcagtggctatccaggtctcacagtttccatggtaacccagtcctccagttcagcaggctaaggggagggcagatggggcctctgggcagagcacaggcgttgtgctgggagcagccgcctcacgagttactgtcatctgtcctctaccggacataataaatgggagaagctttcgggcatgggggaagaggctcccatatgtccctcttcctttctaccccctctctccaccccctccccagctgtctcttctcttcccatcacagttcttgctccctctttcccctcctctatcccagctcatccctcctccgcttaggagcctgcaatgggagtcctctcctccatcgctctcagaatttctctcgtctcctctcccttccccaccattggccactggccatcctctctggctctggattctctctctgaggggctcacagaacctttagcttctctttcttcatccccttccacactcattgttgaagggagaggatggggcagggagggtccttccgggagcccagctctttttctctcactctaaatttcacctgtgtctatccttgcctgccctggagctgtaattcagacccactacttttcttgttcacttttctcacctgcatggcacacaaagacagacagaaccatccatagacacctgagcacacaactaggccacatatacagtaccatacctgggtctcaaaatgatactcctgggttacctgagtatacttaaaaaaacaaagttgcacactgtggtgattaagagtagtgctttaggtggggcgcagtggttcctgcctgtaatcccaacactttgggaggccaaggaggaagtttgagaccagcctgggcaacatagcgagaaccctgtcactacaaaaaaataaaataaaataaattcagatgtaatggcatgcacctggagtcccacctacccaggaggctaaggtggaaggataacttgagcccaggagttcggggatgcagtgagccataatcgtgccactgcattccagcctgggtggcagagtgagaccctgtttcaagttgcagttctaccagttttttgaccatgggtgggttaaaatcaagcctcacttgattgtcagaaaaatggtgaaaataatggtaaccacctactgagaataaatgcatttaaataagcacttagcataaa

SEQ ID NO: 3 - RNA, 3584 nt, Homo sapiens
cgccgggggtgtgtacgccatgagggggaggaagccgagaagcacctcccactgaagccggcggtaaccgccccacgcgagaccacccaccagccaggagcggtcgcaggacccgcagcggcgcgaggccttgtttacaccgactgccgccactgcgttggccaaccgctcctccgtcacgcgcttcgctcgtcacgcatctcgcggcttcctggccggcgggctgcaaacgtcacacgacggccccgcccccttggccagccaactggacagcggagcacgatgattgataggtgcactcctcccagcccagctccagaatggccctgccccccagccccctggccatggaatatgtcaatgactttgacttgatgaagtttgaggtaaagcgggaaccctctgagggccgacctggcccccctacagcctcactgggctccacaccttacagctcagtgcctccttcacccaccttcagtgaaccaggcatggtgggggcaaccgagggcacccggccaggcctggaggagctgtactggctggctaccctgcagcagcagctgggggctggggaggcattggggctgagtcctgaagaggccatggagctgctgcagggtcagggcccagtccctgttgatgggccccatggctactacccagggagcccagaggagacaggagcccagcacgtccagctggcagagcggttttccgacgcggcgctggtctcgatgtctgtgcgggagctaaaccggcagctgcggggctgcgggcgcgacgaggcgctgcggctgaagcagaggcgccgcacgctgaagaaccgcggctacgcgcaggcctgtcgctccaagcggctgcagcagcggcgcgggctggaggccgagcgcgcccgcctggccgcccagctggacgcgctgcgggccgaggtggcccgcctggcccgggagcgcgatctctacaaggctcgctgtgaccggctaacctcgagcggccccgggtccggggacccctcccacctcttcctctgagccgttcagagcaccttgtggtgtagtgggggctgggtggggtggctccgcccaggaggcggctgcacggttctctgcatcgttaccagagcgccttctggtcctagccacgccctgtatgaccgcgcaaatatccccaaagcttttgggtcctcaagtcatgcccgaatttagatgctggtcattttctggagaggggtcccctccccttacgaacacagaaacccagcccacatgactagcacgctgagctctgcagggaccagtgccaggcactggggggtggaagtgtggtgacacagtgaatgggaggtggaggagggttgcagctcccacctcagtttagtttttaattcagggttttcaacctgtaacacattaaagctgtaattagcaatgaggctgtattttcattctgaagcttgtaacctccccattttagcactacagaattttcaagatttcaatatccaacaactagatagattaggacctctatccgagatgctttttccctgcccaaccctgtggccttcagggctcagagcagcaaaggcctgaagagtgagctctgggggttgttggtgtgggttgggagagagctgtgtgcagaagtctggaaacctgggtcctagtcccagctcttccatgggatccccctgtcaccctgagcaaatcagttgcttcctggacttgtgttacttcatctaattctcatgtggattggacgacttctgctccctttccagttctggcatctccccagtatggaagtcccggtggtctccccaagaagtccccaagacaatctcgccaaaggcacctcctatcctgctgcagtttcccagctgcagcctaggcaggggatgcacagcccaggcgaggaagcctggcttctctgtgagcacatacgtgggtcctcggcagctccctccaggctgtctgggcctccagacctgcacagggtgctcctgccacctcccacctctctgagggctgaggtgagacttctcctgggatgacaatttgctgagagagtgcagcttttgtgaattaaacttgaagtccaggcagaattctaatgcaataagctaaatgttcttgcaatttaagaagtgttcattctttatccctgcttcaggctactgtttgtgtgtctgtgtgtgggagagggagagagagggagagagagagagagcaagaaaaagggaagtccccacctggagctgggtggcacatctgttatggagagccaatggaaatggggtggaggggcaggcattggaacagatggatcctccccctctcaccaccaccaccaccaaactcagaagcctcagtggcccagctcggcctgtcagtggctatccaggtctcacagtttccatggtaacccagtcctccagttcagcaggctaaggggagggcagatggggcctctgggcagagcacaggcgttgtgctgggagcagccgcctcacgagttactgtcatctgtcctctaccggacataataaatgggagaagctttcgggcatgggggaagaggctcccatatgtccctcttcctttctaccccctctctccaccccctccccagctgtctcttctcttcccatcacagttcttgctccctctttcccctcctctatcccagctcatccctcctccgcttaggagcctgcaatgggagtcctctcctccatcgctctcagaatttctctcgtctcctctcccttccccaccattggccactggccatcctctctggctctggattctctctctgaggggctcacagaacctttagcttctctttcttcatccccttccacactcattgttgaagggagaggatggggcagggagggtccttccgggagcccagctctttttctctcactctaaatttcacctgtgtctatccttgcctgccctggagctgtaattcagacccactacttttcttgttcacttttctcacctgcatggcacacaaagacagacagaaccatccatagacacctgagcacacaactaggccacatatacagtaccatacctgggtctcaaaatgatactcctgggttacctgagtatacttaaaaaaacaaagttgcacactgtggtgattaagagtagtgctttaggtggggcgcagtggttcctgcctgtaatcccaacactttgggaggccaaggaggaagtttgagaccagcctgggcaacatagcgagaaccctgtcactacaaaaaaataaaataaaataaattcagatgtaatggcatgcacctggagtcccacctacccaggaggctaaggtggaaggataacttgagcccaggagttcggggatgcagtgagccataatcgtgccactgcattccagcctgggtggcagagtgagaccctgtttcaagttgcagttctaccagttttttgaccatgggtgggttaaaatcaagcctcacttgattgtcagaaaaatggtgaaaataatggtaaccacctactgagaataaatgcatttaaataagcacttagcataaa

SEQ ID NO: 4 - RNA, 3708 nt, Homo sapiens
gcctcagagaataaataggagactaagaggacaggcagacaaacaggacgagcctgggggctccaggcctggacggaggaagaagccagctatacggggtttctccatgctggccaagctggtctccaactcctgacctcgtgatccgcctgccttggcctcccaaagtgctggaatttacaggcgtgagccaccacgcccggcctggtcattctttctctccacaatccaatgaggaaactcatctgcccacccaactaccatcataaagtcctcaggtgatttctcaagctatacctccattgggacatctctctccaacctgagaccactggatatcacccacaatgctatatagcacctcaaactcagcatatctatgatgggtgtctgctcagcatccactcgccttttctggtgcactcctcccagcccagctccagaatggccctgccccccagccccctggccatggaatatgtcaatgactttgacttgatgaagtttgaggtaaagcgggaaccctctgagggccgacctggcccccctacagcctcactgggctccacaccttacagctcagtgcctccttcacccaccttcagtgaaccaggcatggtgggggcaaccgagggcacccggccaggcctggaggagctgtactggctggctaccctgcagcagcagctgggggctggggaggcattggggctgagtcctgaagaggccatggagctgctgcagggtcagggcccagtccctgttgatgggccccatggctactacccagggagcccagaggagacaggagcccagcacgtccagctggcagagcggttttccgacgcggcgctggtctcgatgtctgtgcgggagctaaaccggcagctgcggggctgcgggcgcgacgaggcgctgcggctgaagcagaggcgccgcacgctgaagaaccgcggctacgcgcaggcctgtcgctccaagcggctgcagcagcggcgcgggctggaggccgagcgcgcccgcctggccgcccagctggacgcgctgcgggccgaggtggcccgcctggcccgggagcgcgatctctacaaggctcgctgtgaccggctaacctcgagcggccccgggtccggggacccctcccacctcttcctctgagccgttcagagcaccttgtggtgtagtgggggctgggtggggtggctccgcccaggaggcggctgcacggttctctgcatcgttaccagagcgccttctggtcctagccacgccctgtatgaccgcgcaaatatccccaaagcttttgggtcctcaagtcatgcccgaatttagatgctggtcattttctggagaggggtcccctccccttacgaacacagaaacccagcccacatgactagcacgctgagctctgcagggaccagtgccaggcactggggggtggaagtgtggtgacacagtgaatgggaggtggaggagggttgcagctcccacctcagtttagtttttaattcagggttttcaacctgtaacacattaaagctgtaattagcaatgaggctgtattttcattctgaagcttgtaacctccccattttagcactacagaattttcaagatttcaatatccaacaactagatagattaggacctctatccgagatgctttttccctgcccaaccctgtggccttcagggctcagagcagcaaaggcctgaagagtgagctctgggggttgttggtgtgggttgggagagagctgtgtgcagaagtctggaaacctgggtcctagtcccagctcttccatgggatccccctgtcaccctgagcaaatcagttgcttcctggacttgtgttacttcatctaattctcatgtggattggacgacttctgctccctttccagttctggcatctccccagtatggaagtcccggtggtctccccaagaagtccccaagacaatctcgccaaaggcacctcctatcctgctgcagtttcccagctgcagcctaggcaggggatgcacagcccaggcgaggaagcctggcttctctgtgagcacatacgtgggtcctcggcagctccctccaggctgtctgggcctccagacctgcacagggtgctcctgccacctcccacctctctgagggctgaggtgagacttctcctgggatgacaatttgctgagagagtgcagcttttgtgaattaaacttgaagtccaggcagaattctaatgcaataagctaaatgttcttgcaatttaagaagtgttcattctttatccctgcttcaggctactgtttgtgtgtctgtgtgtgggagagggagagagagggagagagagagagagcaagaaaaagggaagtccccacctggagctgggtggcacatctgttatggagagccaatggaaatggggtggaggggcaggcattggaacagatggatcctccccctctcaccaccaccaccaccaaactcagaagcctcagtggcccagctcggcctgtcagtggctatccaggtctcacagtttccatggtaacccagtcctccagttcagcaggctaaggggagggcagatggggcctctgggcagagcacaggcgttgtgctgggagcagccgcctcacgagttactgtcatctgtcctctaccggacataataaatgggagaagctttcgggcatgggggaagaggctcccatatgtccctcttcctttctaccccctctctccaccccctccccagctgtctcttctcttcccatcacagttcttgctccctctttcccctcctctatcccagctcatccctcctccgcttaggagcctgcaatgggagtcctctcctccatcgctctcagaatttctctcgtctcctctcccttccccaccattggccactggccatcctctctggctctggattctctctctgaggggctcacagaacctttagcttctctttcttcatccccttccacactcattgttgaagggagaggatggggcagggagggtccttccgggagcccagctctttttctctcactctaaatttcacctgtgtctatccttgcctgccctggagctgtaattcagacccactacttttcttgttcacttttctcacctgcatggcacacaaagacagacagaaccatccatagacacctgagcacacaactaggccacatatacagtaccatacctgggtctcaaaatgatactcctgggttacctgagtatacttaaaaaaacaaagttgcacactgtggtgattaagagtagtgctttaggtggggcgcagtggttcctgcctgtaatcccaacactttgggaggccaaggaggaagtttgagaccagcctgggcaacatagcgagaaccctgtcactacaaaaaaataaaataaaataaattcagatgtaatggcatgcacctggagtcccacctacccaggaggctaaggtggaaggataacttgagcccaggagttcggggatgcagtgagccataatcgtgccactgcattccagcctgggtggcagagtgagaccctgtttcaagttgcagttctaccagttttttgaccatgggtgggttaaaatcaagcctcacttgattgtcagaaaaatggtgaaaataatggtaaccacctactgagaataaatgcatttaaataagcacttagcataaa

SEQ ID NO: 5 - RNA, 3269 nt, Homo sapiens
cgccgggggtgtgtacgccatgagggggaggaagccgagaagcacctcccactgaagccggcggtaaccgccccacgcgagaccacccaccagccaggagcggtcgcaggacccgcagcggcgcgaggccttgtttacaccgactgccgccactgcgttggccaaccgctcctccgtcacgcgcttcgctcgtcacgcatctcgcggcttcctggccggcgggctgcaaacgtcacacgacggccccgcccccttggccagccaactggacagcggagcacgatgattgataggtgcactcctcccagcccagctccagaatggccctgccccccagccccctggccatggaatatgtcaatgactttgacttgatgaagtttgagctggcagagcggttttccgacgcggcgctggtctcgatgtctgtgcgggagctaaaccggcagctgcggggctgcgggcgcgacgaggcgctgcggctgaagcagaggcgccgcacgctgaagaaccgcggctacgcgcaggcctgtcgctccaagcggctgcagcagcggcgcgggctggaggccgagcgcgcccgcctggccgcccagctggacgcgctgcgggccgaggtggcccgcctggcccgggagcgcgatctctacaaggctcgctgtgaccggctaacctcgagcggccccgggtccggggacccctcccacctcttcctctgagccgttcagagcaccttgtggtgtagtgggggctgggtggggtggctccgcccaggaggcggctgcacggttctctgcatcgttaccagagcgccttctggtcctagccacgccctgtatgaccgcgcaaatatccccaaagcttttgggtcctcaagtcatgcccgaatttagatgctggtcattttctggagaggggtcccctccccttacgaacacagaaacccagcccacatgactagcacgctgagctctgcagggaccagtgccaggcactggggggtggaagtgtggtgacacagtgaatgggaggtggaggagggttgcagctcccacctcagtttagtttttaattcagggttttcaacctgtaacacattaaagctgtaattagcaatgaggctgtattttcattctgaagcttgtaacctccccattttagcactacagaattttcaagatttcaatatccaacaactagatagattaggacctctatccgagatgctttttccctgcccaaccctgtggccttcagggctcagagcagcaaaggcctgaagagtgagctctgggggttgttggtgtgggttgggagagagctgtgtgcagaagtctggaaacctgggtcctagtcccagctcttccatgggatccccctgtcaccctgagcaaatcagttgcttcctggacttgtgttacttcatctaattctcatgtggattggacgacttctgctccctttccagttctggcatctccccagtatggaagtcccggtggtctccccaagaagtccccaagacaatctcgccaaaggcacctcctatcctgctgcagtttcccagctgcagcctaggcaggggatgcacagcccaggcgaggaagcctggcttctctgtgagcacatacgtgggtcctcggcagctccctccaggctgtctgggcctccagacctgcacagggtgctcctgccacctcccacctctctgagggctgaggtgagacttctcctgggatgacaatttgctgagagagtgcagcttttgtgaattaaacttgaagtccaggcagaattctaatgcaataagctaaatgttcttgcaatttaagaagtgttcattctttatccctgcttcaggctactgtttgtgtgtctgtgtgtgggagagggagagagagggagagagagagagagcaagaaaaagggaagtccccacctggagctgggtggcacatctgttatggagagccaatggaaatggggtggaggggcaggcattggaacagatggatcctccccctctcaccaccaccaccaccaaactcagaagcctcagtggcccagctcggcctgtcagtggctatccaggtctcacagtttccatggtaacccagtcctccagttcagcaggctaaggggagggcagatggggcctctgggcagagcacaggcgttgtgctgggagcagccgcctcacgagttactgtcatctgtcctctaccggacataataaatgggagaagctttcgggcatgggggaagaggctcccatatgtccctcttcctttctaccccctctctccaccccctccccagctgtctcttctcttcccatcacagttcttgctccctctttcccctcctctatcccagctcatccctcctccgcttaggagcctgcaatgggagtcctctcctccatcgctctcagaatttctctcgtctcctctcccttccccaccattggccactggccatcctctctggctctggattctctctctgaggggctcacagaacctttagcttctctttcttcatccccttccacactcattgttgaagggagaggatggggcagggagggtccttccgggagcccagctctttttctctcactctaaatttcacctgtgtctatccttgcctgccctggagctgtaattcagacccactacttttcttgttcacttttctcacctgcatggcacacaaagacagacagaaccatccatagacacctgagcacacaactaggccacatatacagtaccatacctgggtctcaaaatgatactcctgggttacctgagtatacttaaaaaaacaaagttgcacactgtggtgattaagagtagtgctttaggtggggcgcagtggttcctgcctgtaatcccaacactttgggaggccaaggaggaagtttgagaccagcctgggcaacatagcgagaaccctgtcactacaaaaaaataaaataaaataaattcagatgtaatggcatgcacctggagtcccacctacccaggaggctaaggtggaaggataacttgagcccaggagttcggggatgcagtgagccataatcgtgccactgcattccagcctgggtggcagagtgagaccctgtttcaagttgcagttctaccagttttttgaccatgggtgggttaaaatcaagcctcacttgattgtcagaaaaatggtgaaaataatggtaaccacctactgagaataaatgcatttaaataagcacttagcataaa